(19) United States
(12) Patent Application Publication
(10) Pub. No.: US 2025/0316032 A1
(43) Pub. Date: Oct. 9, 2025

(54) USER INTERFACES FOR EXTENDED REALITY EXPERIENCES INCLUDING A LIVE FEED FROM ONE OR MORE EXTERNAL SENSORS

(71) Applicant: Apple Inc., Cupertino, CA (US)

(72) Inventors: Omar R. KHAN, San Ramon, CA (US); Angel Suet Yan CHEUNG, San Francisco, CA (US); Swapnil MENGADE, Sunnyvale, CA (US); Yunqi HU, Sunnyvale, CA (US); Karen N. WONG, Sunnyvale, CA (US); Jue WANG, Sunnyvale, CA (US); Jonathan PERRON, Felton, CA (US); Esteban CASTELLANOS, Los Angeles, CA (US); Matthew L. STERN, Hermosa Beach, CA (US); David SCOTT, Oakland, CA (US); Michelle CHUA, Seattle, WA (US)

(21) Appl. No.: 19/169,983

(22) Filed: Apr. 3, 2025

Related U.S. Application Data
(60) Provisional application No. 63/631,939, filed on Apr. 9, 2024.
(60) Provisional application No. 63/699,097, filed on Sep. 25, 2024.
(60) Provisional application No. 63/699,100, filed on Sep. 25, 2024.

(51) Int. Cl. G06T 19/00; A61B 1/313

(52) U.S. Cl. CPC G06T 19/006; A61B 1/313

(57) ABSTRACT

An electronic device displays a widget dashboard user interface in a three-dimensional environment, displays a representation of a physical tool to as guidance for indicating a location of the physical tool relative to a location associated with video feed, displays suggestions for changing a pose of a camera to a predetermined pose based on image data detected while the camera previously had the predetermined pose, displays a live camera feed and image data and scrubs through the image data in accordance with changes to a pose of the camera relative to a physical object, displays live stereoscopic camera feed with special effects, detects and responds to inputs for virtually annotating portions of objects, and/or displays models of objects and detects and responds to input for rotating and/or viewing the model from different depth positions within the model.

### CROSS REFERENCE TO RELATED APPLICATIONS

This application claims the benefit of U.S. Provisional Application No. 63/631,939, filed Apr. 9, 2024, U.S. Provisional Application No. 63/699,097, filed Sep. 25, 2024, and U.S. Provisional Application No. 63/699,100, filed Sep. 25, 2024, the contents of which are herein incorporated by reference in their entireties for all purposes.

### FIELD OF THE DISCLOSURE

The present disclosure relates generally to computer systems that provide computer-generated experiences, including, but not limited to, electronic devices that provide virtual reality and mixed reality experiences via a display.

### BACKGROUND OF THE DISCLOSURE

Augmented reality environments include at least some virtual elements that replace or augment the physical world. Input devices, such as cameras, controllers, joysticks, touch-sensitive surfaces, and touch-screen displays for computer systems and other electronic computing devices are often used to interact with virtual/augmented reality environments. Example virtual elements include virtual objects, such as digital images, video, text, icons, and control elements such as buttons and other graphics.

### SUMMARY OF THE DISCLOSURE

Some examples of the disclosure are directed to an electronic device displaying a widget dashboard user interface in a three-dimensional environment.

Some examples of the disclosure are directed to an electronic device displaying a representation of a physical tool for indicating a location of the physical tool relative to a location associated with video feed.

Some examples of the disclosure are directed to an electronic device displaying indications of proximities of physical tools relative to one or more surfaces of a physical object.

Some examples of the disclosure are directed to an electronic device displaying suggestions for changing a pose of a camera to a predetermined pose relative to a physical object.

Some examples of the disclosure are directed to an electronic device displaying one or more user interface elements overlaid on an external view of a physical object and/or on an internal view of the physical object captured by the camera.

Some examples of the disclosure are directed to an electronic device displaying a live camera feed and image data, and scrubbing through the image data in accordance with changes to a pose of the camera relative to a physical object.

Some examples of the disclosure are directed to an electronic device displaying live stereoscopic camera feed with special effects.

Some examples of the disclosure are directed to an electronic device detecting and responding to inputs for annotating portions of objects.

Some examples of the disclosure are directed to an electronic device displaying a 3D model of an object, and detecting and responding to inputs for rotating and/or viewing the model from different depth positions within the model in accordance with some examples.

The full descriptions of these examples are provided in the Drawings and the Detailed Description, and it is understood that this Summary does not limit the scope of the disclosure in any way.

### BRIEF DESCRIPTION OF THE DRAWINGS

For improved understanding of the various examples described herein, reference should be made to the Detailed Description below along with the following drawings. Like reference numerals often refer to corresponding parts throughout the drawings.

FIG. 1 illustrates an electronic device presenting an extended reality environment according to some examples of the disclosure.

FIG. 2 illustrates a block diagram of an example architecture for a system according to some examples of the disclosure.

FIGS. 3A-3H illustrate examples of a computer systems displaying user interfaces and/or a dashboard of widgets according to some examples of the disclosure.

FIG. 3I is a flow diagram illustrating a method for displaying a widget dashboard user interface according to some examples of the disclosure.

FIGS. 4A-4G generally illustrate examples of an electronic device displaying a representation of a physical tool in accordance with satisfaction of criteria according to some examples of the disclosure.

FIG. 4H is a flow diagram illustrating a method for displaying a representation of a physical tool as guidance for indicating a location of the physical tool relative to a location associated with video feed according to some examples of the disclosure.

FIGS. 5A-5G illustrate examples of an electronic device displaying suggestions for changing a pose of a camera to predetermined pose based on image data according to some examples of the disclosure.

FIG. 5H is a flow diagram illustrating a method for displaying a visual indication suggesting changing a pose of a camera according to some examples of the disclosure.

FIGS. 6A-6E illustrate examples of an electronic device scrubbing through image data while displaying a live camera feed user interface according to some examples of the disclosure.

FIG. 6F is a flow diagram illustrating a method for updating display of user interfaces in response to detecting camera movement according to some examples of the disclosure.

FIGS. 7A-7C illustrate examples of an electronic device displaying live stereoscopic camera feed with special effects according to some examples of the disclosure.

FIG. 7D is a flow diagram illustrating a method for displaying live stereoscopic camera feed with special effects according to some examples of the disclosure.

FIGS. 8A-8L illustrate examples of an electronic device presenting a live camera feed user interface including video feed from a camera from inside a physical object, and virtually annotating in the live camera feed user interface according to some examples of the disclosure.

FIG. 8M is a flow diagram illustrating a method for displaying an annotation in a user interface that includes a render of camera feed showing a portion of an object, and for moving the annotation in response to detecting an event corresponding to relative movement between the camera and the portion of the object according to some examples of the disclosure.

FIGS. 9A-9K illustrate examples of an electronic device displaying a 3D model of an object, and detecting and responding to inputs corresponding to requests for rotating and/or viewing the model from different depth positions within the model in accordance with some examples.

FIG. 9L is a flow diagram illustrating a method for displaying a 3D model of an object, and detecting and responding to movement component of a first selection input according to some examples of the disclosure.

### DETAILED DESCRIPTION

In the following description of examples, reference is made to the accompanying drawings which form a part hereof, and in which it is shown by way of illustration specific examples that can be practiced. It is to be understood that other examples can be used and structural changes can be made without departing from the scope of the disclosed examples.

Some examples of the disclosure are directed to an electronic device displaying a widget dashboard user interface in a three-dimensional environment.

Some examples of the disclosure are directed to an electronic device displaying a representation of a physical tool to as guidance for indicating a location of the physical tool relative to a location associated with video feed.

Some examples of the disclosure are directed to an electronic device displaying indications of proximities of physical tools relative to one or more surfaces of a physical object.

Some examples of the disclosure are directed to an electronic device displaying suggestions for changing a pose of a camera to a predetermined pose based on image data detected while the camera previously had the predetermined pose.

Some examples of the disclosure are directed to an electronic device displaying one or more user interface elements overlaid on an external view of a physical object and/or on an internal view of the physical object captured by the camera.

Some examples of the disclosure are directed to an electronic device displaying a live camera feed and image data, and scrubbing through the image data in accordance with changes to a pose of the camera relative to a physical object.

Some examples of the disclosure are directed to an electronic device displaying live stereoscopic camera feed with special effects.

Some examples of the disclosure are directed to an electronic device detecting and responding to inputs for annotating portions of objects.

Some examples of the disclosure are directed to an electronic device displaying a 3D model of an object, and detecting and responding to inputs for rotating and/or viewing the model from different depth positions within the model in accordance with some examples.

The user interfaces, methods, techniques, and computer systems described herein can be used in a variety of contexts, including contexts that involve camera-guided operations or procedures (e.g., drilling operations, manufacturing operations, fabrication operations, and/or other camera-assisted operations). For example, in some circumstances, cameras are used in engineering operations, such that data from cameras guide a user of a system or a system (e.g., such an artificial intelligence assisted system) in performing one or more operations. Further, some operations in which present examples are applicable is in medical operations, such as with camera-guided surgeries. For example, present examples provide for camera-guided surgeries. Although primarily described in the context of camera-guided surgery, it is understood that the disclosure here is not limited to camera-guided surgery or medical context.

Note that although some of the present discussion is provided in the context of a surgical procedure, the examples provided are likewise applicable to other contexts, such as engineering contexts and/or other contexts. As such, the described and/or illustrated examples are not intended to be limited to surgical procedures, but are applicable to nonsurgical and/or nonmedical contexts. Further, note that the various examples described above can be combined with any other examples described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the subject matter herein.

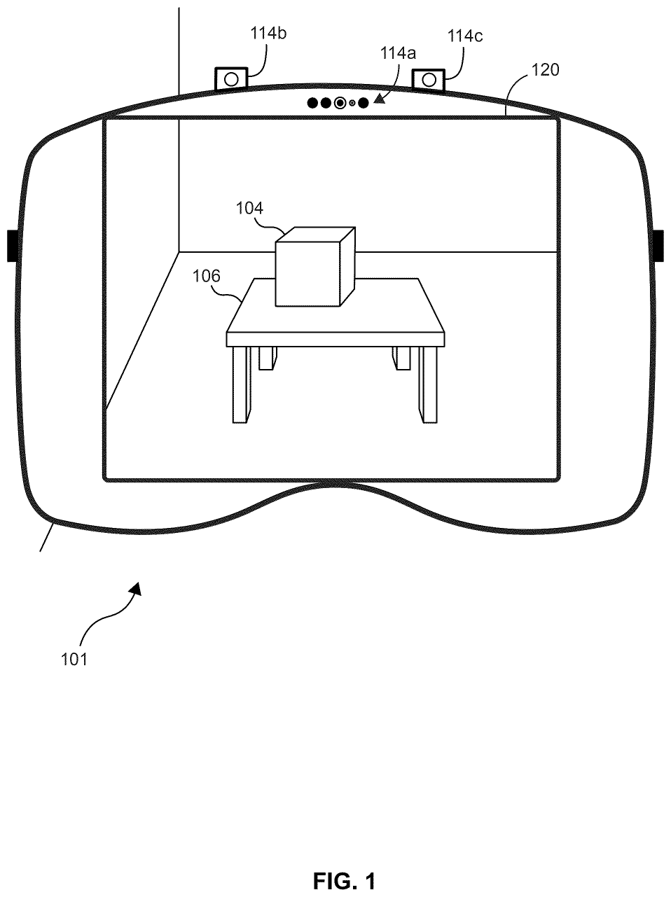

FIG. 1 illustrates an electronic device 101 (e.g., a computer system) presenting an extended reality (XR) environment (e.g., a computer-generated environment optionally including representations of physical and/or virtual objects) according to some examples of the disclosure.

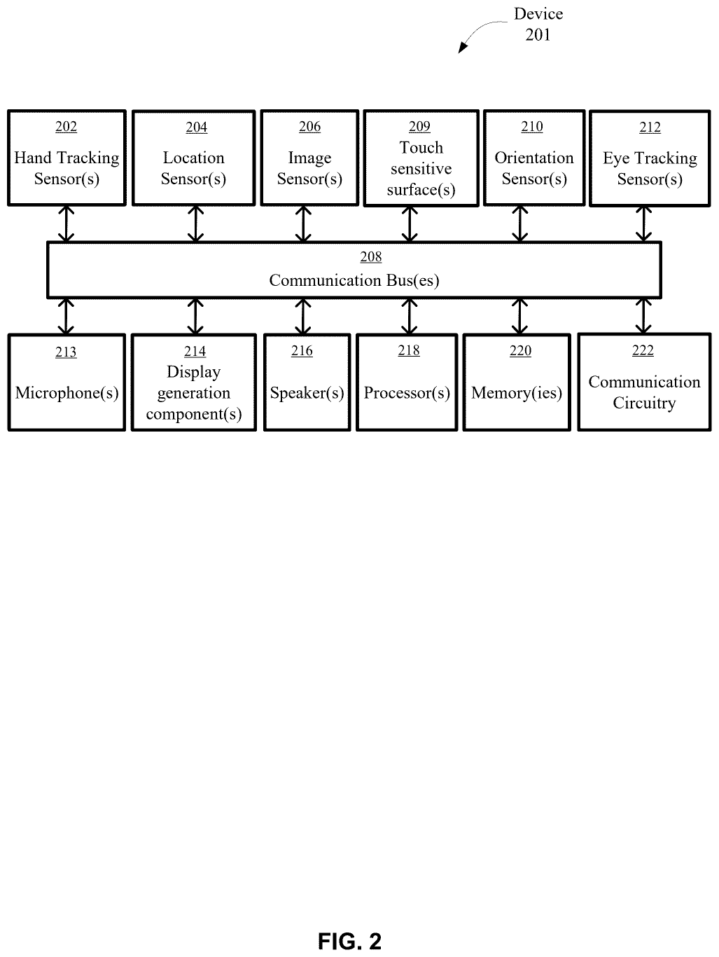

In some examples, as shown in FIG. 1, computer system 101 is a head-mounted display or other head-mountable device configured to be worn on a head of a user of the electronic device 101. Examples of computer system 101 are described below with reference to the architecture block diagram of FIG. 2. As shown in FIG. 1, computer system 101 and table 106 are located in a physical environment. The physical environment may include physical features such as a physical surface (e.g., floor, walls) or a physical object (e.g., table, lamp, etc.). In some examples, computer system 101 may be configured to detect and/or capture images of physical environment including table 106 (illustrated in the field of view of computer system 101).

In some examples, as shown in FIG. 1, computer system 101 includes one or more internal image sensors 114a oriented towards a face of the user (e.g., eye tracking cameras described below with reference to FIG. 2). In some examples, internal image sensors 114a are used for eye tracking (e.g., detecting a gaze of the user). Internal image sensors 114a are optionally arranged on the left and right portions of display 120 to enable eye tracking of the user's left and right eyes. In some examples, computer system 101 also includes external image sensors 114b and 114c facing outwards from the user to detect and/or capture the physical environment of the electronic device 101 and/or movements of the user's hands or other body parts.

In some examples, display 120 has a field of view visible to the user (e.g., that may or may not correspond to a field of view of external image sensors 114b and 114c). Because display 120 is optionally part of a head-mounted device, the field of view of display 120 is optionally the same as or similar to the field of view of the user's eyes. In other examples, the field of view of display 120 may be smaller than the field of view of the user's eyes. In some examples, computer system 101 may be an optical see-through device in which display 120 is a transparent or translucent display through which portions of the physical environment may be directly viewed. In some examples, display 120 may be included within a transparent lens and may overlap all or only a portion of the transparent lens. In other examples, computer system may be a video-passthrough device in which display 120 is an opaque display configured to display images of the physical environment captured by external image sensors 114b and 114c.

In some examples, in response to a trigger, the electronic device 101 may be configured to display a virtual object 104 in the XR environment represented by a cube illustrated in FIG. 1, which is not present in the physical environment, but is displayed in the XR environment positioned on the top of real-world table 106 (or a representation thereof). Optionally, virtual object 104 can be displayed on the surface of the table 106 in the XR environment displayed via the display 120 of the electronic device 101 in response to detecting the planar surface of table 106 in the physical environment 100.

It should be understood that virtual object 104 is a representative virtual object and one or more different virtual objects (e.g., of various dimensionality such as two-dimensional or other three-dimensional virtual objects) can be included and rendered in a three-dimensional XR environment. For example, the virtual object can represent an application or a user interface displayed in the XR environment. In some examples, the virtual object can represent content corresponding to the application and/or displayed via the user interface in the XR environment. In some examples, the virtual object 104 is optionally configured to be interactive and responsive to user input (e.g., air gestures, such as air pinch gestures, air tap gestures, and/or air touch gestures), such that a user may virtually touch, tap, move, rotate, or otherwise interact with, the virtual object 104.

In some examples, displaying an object in a three-dimensional environment may include interaction with one or more user interface objects in the three-dimensional environment. For example, initiation of display of the object in the three-dimensional environment can include interaction with one or more virtual options/affordances displayed in the three-dimensional environment. In some examples, a user's gaze may be tracked by the computer system as an input for identifying one or more virtual options/affordances targeted for selection when initiating display of an object in the three-dimensional environment. For example, gaze can be used to identify one or more virtual options/affordances targeted for selection using another selection input. In some examples, a virtual option/affordance may be selected using hand-tracking input detected via an input device in communication with the computer system. In some examples, objects displayed in the three-dimensional environment may be moved and/or reoriented in the three-dimensional environment in accordance with movement input detected via the input device.

In the discussion that follows, a computer system that is in communication with a display generation component and one or more input devices is described. It should be understood that the computer system optionally is in communication with one or more other physical user-interface devices, such as a touch-sensitive surface, a physical keyboard, a mouse, a joystick, a hand tracking device, an eye tracking device, a stylus, etc. Further, as described above, it should be understood that the described computer system, display and touch-sensitive surface are optionally distributed amongst two or more devices. Therefore, as used in this disclosure, information displayed on the computer system or by the computer system is optionally used to describe information outputted by the computer system for display on a separate display device (touch-sensitive or not). Similarly, as used in this disclosure, input received on the computer system (e.g., touch input received on a touch-sensitive surface of the computer system, or touch input received on the surface of a stylus) is optionally used to describe input received on a separate input device, from which the computer system receives input information.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, a television channel browsing application, and/or a digital video player application.

FIG. 2 illustrates a block diagram of an example architecture for a computer system device 201 according to some examples of the disclosure.

In some examples, computer system 201 includes one or more computer systems. For example, the computer system 201 may be a portable device, an auxiliary device in communication with another device, a head-mounted display, etc., respectively. In some examples, computer system 201 corresponds to computer system 101 described above with reference to FIG. 1.

As illustrated in FIG. 2, the computer system 201 optionally includes various sensors, such as one or more hand tracking sensors 202, one or more location sensors 204, one or more image sensors 206 (optionally corresponding to internal image sensors 114a and/or external image sensors 114b and 114c in FIG. 1), one or more touch-sensitive surfaces 209, one or more motion and/or orientation sensors 210, one or more eye tracking sensors 212, one or more microphones 213 or other audio sensors, one or more body tracking sensors (e.g., torso and/or head tracking sensors), one or more display generation components 214, optionally corresponding to display 120 in FIG. 1, one or more speakers 216, one or more processors 218, one or more memories 220, and/or communication circuitry 222. One or more communication buses 208 are optionally used for communication between the above-mentioned components of computer systems 201.

Communication circuitry 222 optionally includes circuitry for communicating with computer systems, networks, such as the Internet, intranets, a wired network and/or a wireless network, cellular networks, and wireless local area networks (LANs). Communication circuitry 222 optionally includes circuitry for communicating using near-field communication (NFC) and/or short-range communication, such as Bluetooth®.

Processor(s) 218 include one or more general processors, one or more graphics processors, and/or one or more digital signal processors. In some examples, memory 220 is a non-transitory computer-readable storage medium (e.g., flash memory, random access memory, or other volatile or non-volatile memory or storage) that stores computer-readable instructions configured to be executed by processor(s) 218 to perform the techniques, processes, and/or methods described below. In some examples, memory 220 can include more than one non-transitory computer-readable storage medium. A non-transitory computer-readable storage medium can be any medium (e.g., excluding a signal) that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on compact disc (CD), digital versatile disc (DVD), or Blu-ray technologies, as well as persistent solid-state memory such as flash, solid-state drives, and the like.

In some examples, display generation component(s) 214 include a single display (e.g., a liquid-crystal display (LCD), organic light-emitting diode (OLED), or other types of display). In some examples, display generation component(s) 214 includes multiple displays. In some examples, display generation component(s) 214 can include a display with touch capability (e.g., a touch screen), a projector, a holographic projector, a retinal projector, a transparent or translucent display, etc. In some examples, computer system 201 includes touch-sensitive surface(s) 209, respectively, for receiving user inputs, such as tap inputs and swipe inputs or other gestures. In some examples, display generation component(s) 214 and touch-sensitive surface(s) 209 form touch-sensitive display(s) (e.g., a touch screen integrated with computer system 201 or external to computer system 201 that is in communication with computer system 201).

Computer system 201 optionally includes image sensor(s) 206. Image sensors(s) 206 optionally include one or more visible light image sensors, such as charged coupled device (CCD) sensors, and/or complementary metal-oxide-semiconductor (CMOS) sensors operable to obtain images of physical objects from the real-world environment. Image sensor(s) 206 also optionally include one or more infrared (IR) sensors, such as a passive or an active IR sensor, for detecting infrared light from the real-world environment. For example, an active IR sensor includes an IR emitter for emitting infrared light into the real-world environment. Image sensor(s) 206 also optionally include one or more cameras configured to capture movement of physical objects in the real-world environment. Image sensor(s) 206 also optionally include one or more depth sensors configured to detect the distance of physical objects from computer system 201. In some examples, information from one or more depth sensors can allow the device to identify and differentiate objects in the real-world environment from other objects in the real-world environment. In some examples, one or more depth sensors can allow the device to determine the texture and/or topography of objects in the real-world environment.

In some examples, computer system 201 uses CCD sensors, event cameras, and depth sensors in combination to detect the physical environment around computer system 201. In some examples, image sensor(s) 206 include a first image sensor and a second image sensor. The first image sensor and the second image sensor work in tandem and are optionally configured to capture different information of physical objects in the real-world environment. In some examples, the first image sensor is a visible light image sensor and the second image sensor is a depth sensor. In some examples, computer system 201 uses image sensor(s) 206 to detect the position and orientation of computer system 201 and/or display generation component(s) 214 in the real-world environment. For example, computer system 201 uses image sensor(s) 206 to track the position and orientation of display generation component(s) 214 relative to one or more fixed objects in the real-world environment.

In some examples, computer system 201 includes microphone(s) 213 or other audio sensors. Computer system 201 optionally uses microphone(s) 213 to detect sound from the user and/or the real-world environment of the user. In some examples, microphone(s) 213 includes an array of microphones (a plurality of microphones) that optionally operate in tandem, such as to identify ambient noise or to locate the source of sound in space of the real-world environment.

Computer system 201 includes location sensor(s) 204 for detecting a location of computer system 201 and/or display generation component(s) 214. For example, location sensor(s) 204 can include a global positioning system (GPS) receiver that receives data from one or more satellites and allows computer system 201 to determine the device's absolute position in the physical world.

Computer system 201 includes orientation sensor(s) 210 for detecting orientation and/or movement of computer system 201 and/or display generation component(s) 214. For example, computer system 201 uses orientation sensor(s) 210 to track changes in the position and/or orientation of computer system 201 and/or display generation component(s) 214, such as with respect to physical objects in the real-world environment. Orientation sensor(s) 210 optionally include one or more gyroscopes and/or one or more accelerometers.

Computer system 201 includes hand tracking sensor(s) 202 and/or eye tracking sensor(s) 212 (and/or other body tracking sensor(s), such as leg, torso and/or head tracking sensor(s)), in some examples. Hand tracking sensor(s) 202 are configured to track the position/location of one or more portions of the user's hands, and/or motions of one or more portions of the user's hands with respect to the extended reality environment, relative to the display generation component(s) 214, and/or relative to another defined coordinate system. Eye tracking sensor(s) 212 are configured to track the position and movement of a user's gaze (eyes, face, or head, more generally) with respect to the real-world or extended reality environment and/or relative to the display generation component(s) 214. In some examples, hand tracking sensor(s) 202 and/or eye tracking sensor(s) 212 are implemented together with the display generation component(s) 214. In some examples, the hand tracking sensor(s) 202 and/or eye tracking sensor(s) 212 are implemented separate from the display generation component(s) 214.

In some examples, the hand tracking sensor(s) 202 (and/or other body tracking sensor(s), such as leg, torso and/or head tracking sensor(s)) can use image sensor(s) 206 (e.g., one or more IR cameras, three-dimensional (3D) cameras, depth cameras, etc.) that capture three-dimensional information from the real-world including one or more body parts (e.g., hands, legs, torso, or head of a human user). In some examples, the hands can be resolved with sufficient resolution to distinguish fingers and their respective positions. In some examples, one or more image sensors 206 are positioned relative to the user to define a field of view of the image sensor(s) 206 and an interaction space in which finger/hand position, orientation and/or movement captured by the image sensors are used as inputs (e.g., to distinguish from a user's resting hand or other hands of other persons in the real-world environment). Tracking the fingers/hands for input (e.g., gestures, touch, tap, etc.) can be advantageous in that it does not require the user to touch, hold or wear any sort of beacon, sensor, or other marker.

In some examples, eye tracking sensor(s) 212 includes at least one eye tracking camera (e.g., infrared (IR) cameras) and/or illumination sources (e.g., IR light sources, such as LEDs) that emit light towards a user's eyes. The eye tracking cameras may be pointed towards a user's eyes to receive reflected IR light from the light sources directly or indirectly from the eyes. In some examples, both eyes are tracked separately by respective eye tracking cameras and illumination sources, and a focus/gaze can be determined from tracking both eyes. In some examples, one eye (e.g., a dominant eye) is tracked by one or more respective eye tracking cameras/illumination sources.

Computer system 201 is not limited to the components and configuration of FIG. 2, but can include fewer, other, or additional components in multiple configurations. In some examples, computer system 201 can be implemented between two computer systems (e.g., as a system). In some such examples, each of (or more) computer system may each include one or more of the same components discussed above, such as various sensors, one or more display generation components, one or more speakers, one or more processors, one or more memories, and/or communication circuitry. A person or persons using computer system 201, is optionally referred to herein as a user or users of the device.

Attention is now directed towards a three-dimensional environment presented at a computer system (e.g., corresponding to computer system 101) which includes displayed image sensor data, and towards systems and methods for displaying widgets in a three-dimensional environment.

Generally, widgets are user interface elements that include information and/or one or more tools that let a user perform tasks and/or provide access to information. Widgets can perform a variety of tasks, including without limitation, communicating with a remote server to provide information to the user (e.g., weather report, patient information), providing commonly needed functionality (e.g., a calculator, initiating a voice or video call), or acting as an information repository (e.g., a notebook, summary of surgery notes). In some examples, widgets can be displayed and accessed through an environment referred to as a “unified interest layer,” “dashboard layer,” “dashboard environment,” or “dashboard.”

Some examples of the disclosure are directed to a method that is performed at a computer system in communication with one or more displays and one or more input devices, including a camera and one or more sensors, different from the camera. The method includes while a physical object is visible via the one or more displays, displaying, via the one or more displays, a widget dashboard user interface, including a first widget including live camera feed from the camera; and one or more second widgets including one or more indications of the physical object, wherein the one or more indications of the physical objects is based data from the one or more sensors. Some examples of the disclosure are directed to a computer system that performs the above-recited method. Some examples of the disclosure are directed to a non-transitory computer readable storage medium storing one or more programs, the one or more programs comprising instructions, which when executed by one or more processors of computer system, cause the computer system to perform the above-recited method.

For example, present examples provide for camera-guided surgeries. Although primarily described in the context of camera-guided surgery, it is understood that the disclosure here is not limited to camera-guided surgery or medical context. In a camera-guided surgery, in some examples, a computer system displays a dashboard widget user interface, including a first widget showing a live camera feed from a surgical camera. In some examples, the dashboard of widgets is displayed in an extended reality environment via one or more displays that comprise a head-mounted display system. In some examples, the dashboard of widgets includes data of the patient. In some examples, while the dashboard of widgets is displayed, a computer system presents one or more portions of a physical environment of the computer system, such as a portion of the physical environment that includes a patient. In some examples, the dashboard of widgets is customizable in location (two-dimensional or three-dimensional coordinate), orientation, size, and/or other characteristics. Additionally or alternatively, the dashboard of widgets is a customizable arrangement of widgets. Additionally or alternatively the displayed widgets are customizable (e.g., the dashboard of widget can include different widgets in response to user input). The customization is optionally implemented prior to a procedure and/or the customization can be adjusted using gestures during the procedure. In some examples, the dashboard of widgets is arranged in the field of view of the user such that the user of the computer system does not have to rotate the user's head and/or torso to undesirable angles (e.g., 30, 40, 45 degrees, or another undesirable angle) to view the dashboard of widgets during the surgical operation. In some examples, the dashboard of widgets includes a widget for controlling an environment setting of the operating room and/or of the environment that is displayed to the user of the computer system. For example, the user of the computer system is optionally a surgeon, and the computer system optionally displays a user interface element for controlling an amount of passthrough dimming of the environment while performing the surgery on the patient. In some examples, the user interface element controls the passthrough dimming of the environment of the surgeon, without changing a lighting setting for other personnel in the operating room. As such, various surgical personnel can customize settings of the environment, without requiring a change in setting of the physical environment.

Medical providers may perform multiple tasks on the same or different patients throughout a given day. It is desirable for medical providers to have access to patient information before, during, and/or after interacting with a patient. For example, a medical provider would benefit from viewing patient information captured by electronic equipment that monitors the patient, and/or from files generated by an electronic device. In some circumstances, a medical provider is tasked with performing one or more medical operations, such as a surgery, on the patient. In one such operation, a medical provider is tasked with performing a camera-guided surgical procedure (e.g., a laparoscopic surgery) on a patient.

In some operating rooms in which a camera-guided surgery is being performed, a first surgical assistant may hold and/or orient a camera inside of the patient and a second surgical assistant may prepare, hold and/or be on standby to access one or more tools (e.g., surgical instruments and/or electronic devices) and/or may assist with other environmental settings associated with the operating room, such as changing a level of lighting in the operating room environment. In addition, in some operating rooms, one or more physical displays are arranged and may display camera feed from the surgical camera in order to guide the surgeon and/or surgical assistants during the surgical procedure. Further, in some operating rooms, the one or more physical displays may display other data relating to the patient and may be arranged at different locations in the physical locations. Sometimes, the one or more physical displays are physically moved by the surgical personnel, which may increase an amount of time associated with the surgical procedure. Sometimes, when the one or more physical displays are arranged at different locations in the operating room, surgical personnel (e.g., the surgeon) may have to rotate their heads and/or torsos to uncomfortable positions while also performing other tasks related to the surgery in order to view the data that is displayed on the physical displays, which may increase an amount of time associated with the surgical procedure and may increase bodily discomfort of the surgical personnel. Furthermore, including multiple physical displays consumes more and more physical space in the operating room. Thus, systems, user interfaces, and methods that assist surgical personnel with viewing data and provide personalized control of environmental settings in the operating room during surgical operations (e.g., in the operating room) optimally results in better surgical outcomes (e.g., faster surgical procedures), reduces discomfort of surgical personnel, and reduces a need for viewing multiple physical displays during a surgical operations.

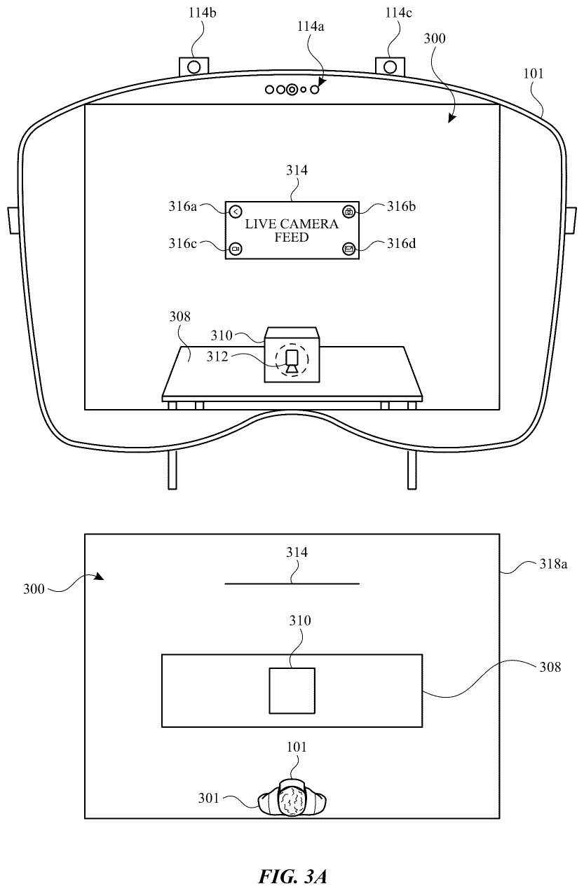

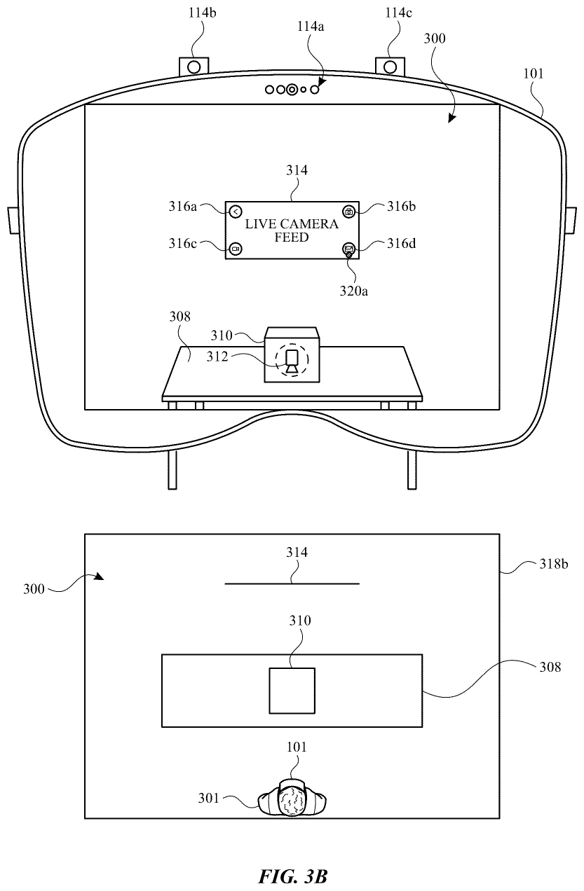

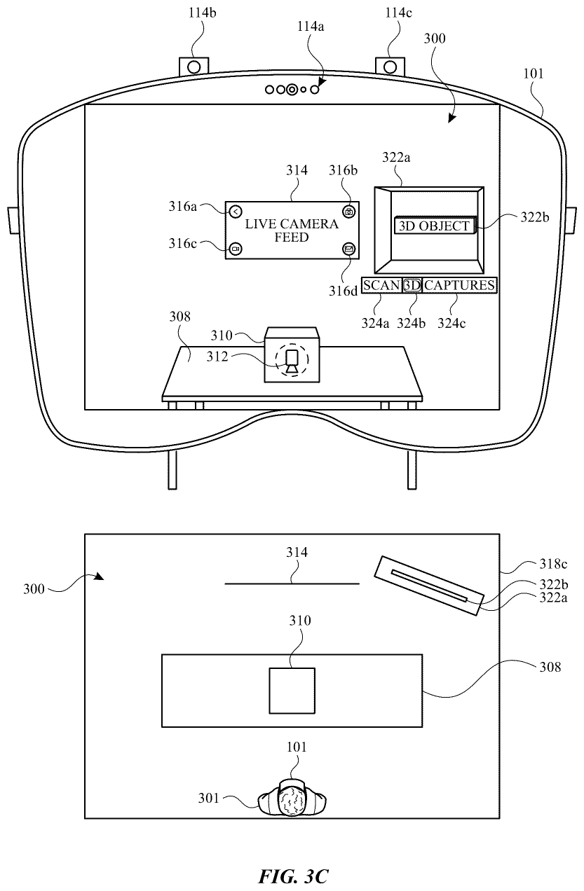

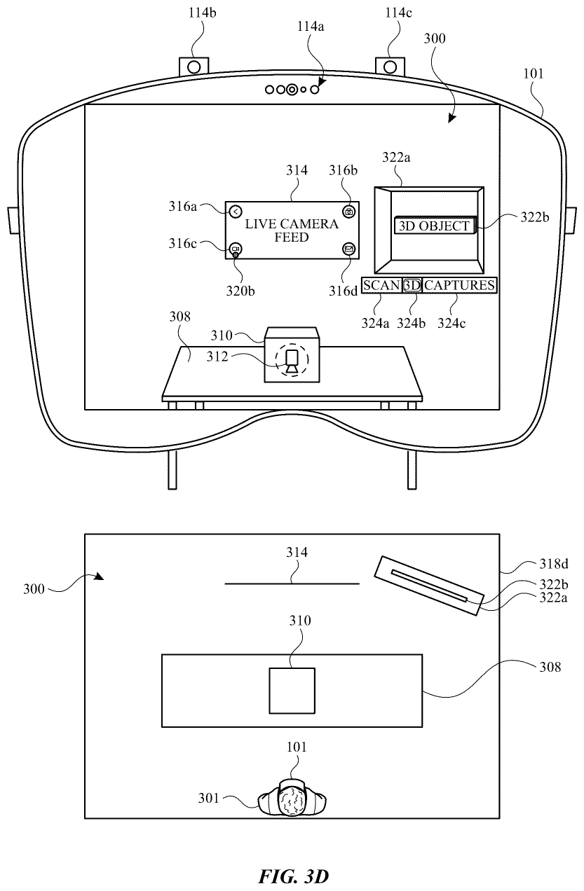

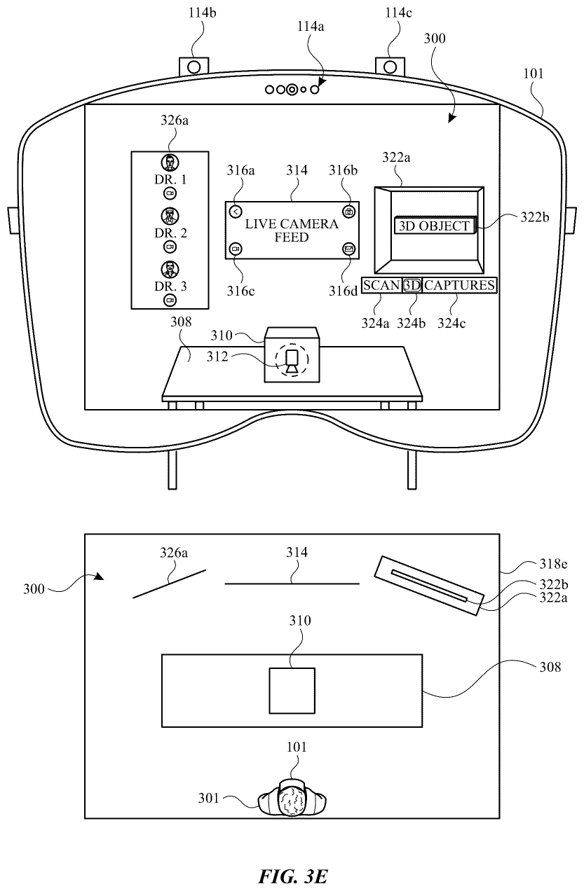

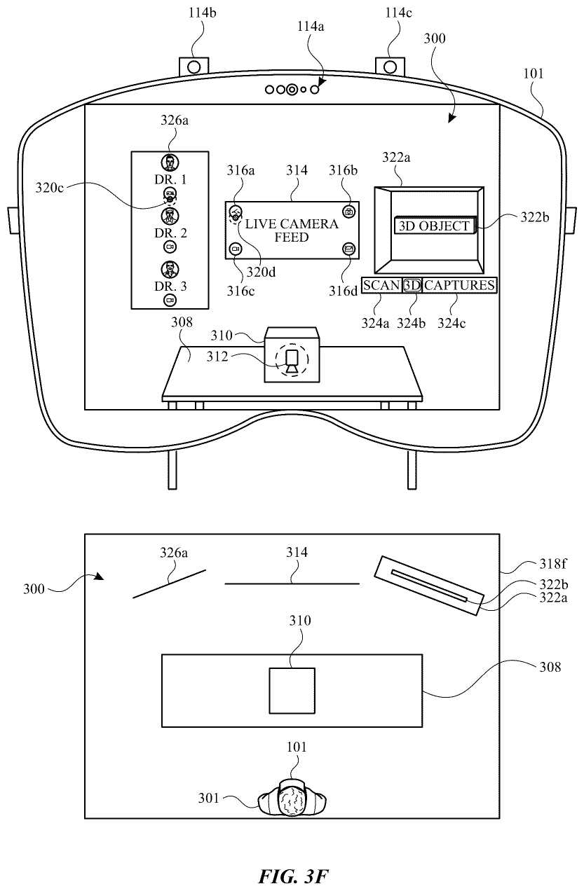

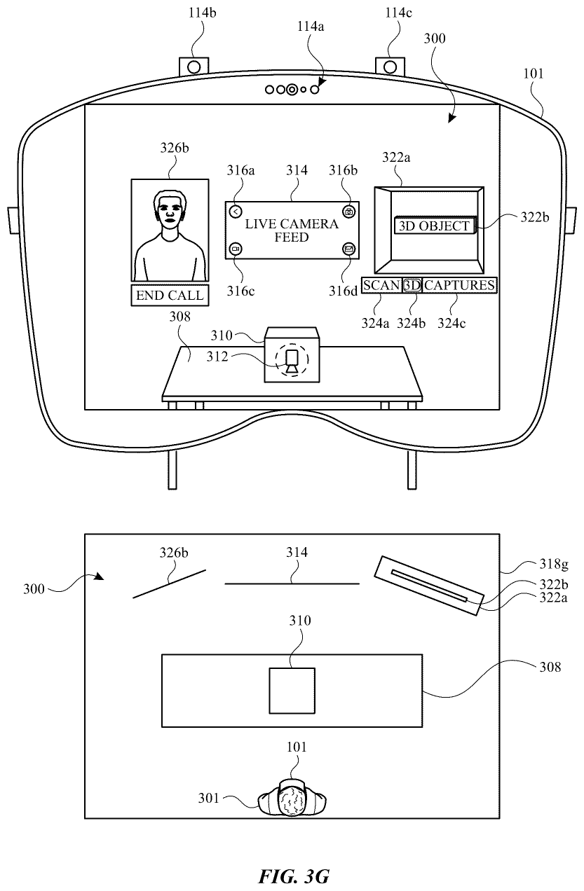

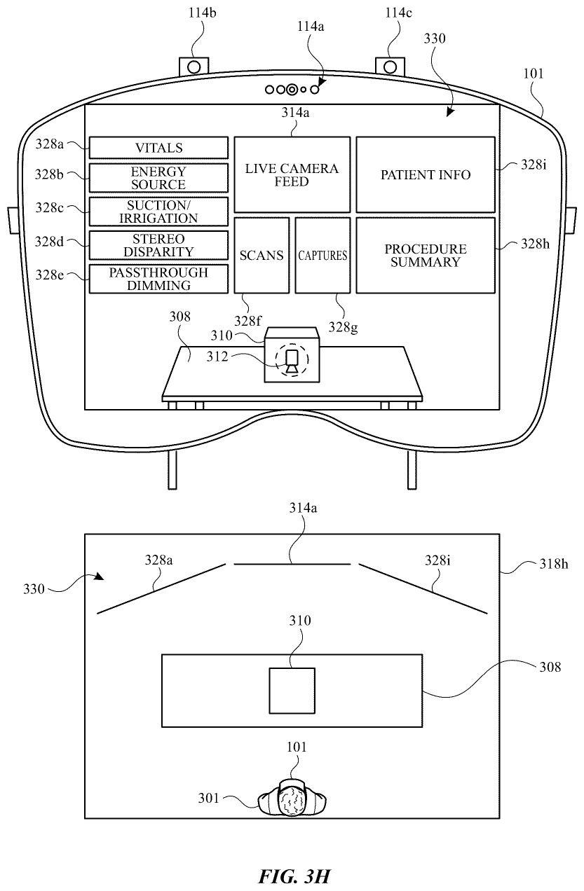

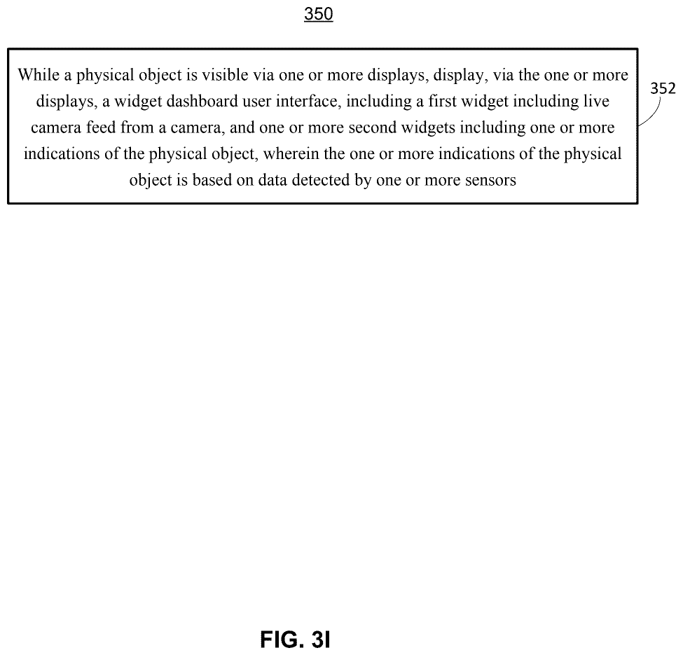

FIGS. 3A-3H illustrate examples of a computer systems displaying user interfaces and/or a dashboard of widgets according to some examples of the disclosure. Although the described context of FIGS. 3A-3H is relative to a surgical operating room including a surgeon (e.g., user 301 of computer system 101) and a patient (e.g., whose body is object 310), the present examples are applicable to even nonsurgical contexts, such as engineering contexts and/or other nonmedical and/or nonsurgical contexts. For the purpose of illustration, FIGS. 3A-3H include respective top-down views 318a-318h of the three-dimensional environment 300 that indicate the positions of various objects (e.g., real and/or virtual objects) in the three-dimensional environment 300 in a horizontal dimension and a depth dimension. The top-down view of the three-dimensional environment 300 further includes an indication of the viewpoint of the user 301 of the electronic device 101. For example, in FIG. 3A, the electronic device 101 displays the view of the three-dimensional environment 300 visible through the display 120 from the viewpoint of the user 301 illustrated in the top-down view 318a of the three-dimensional environment 300.

FIG. 3A illustrates an electronic device 101 displaying a live camera feed user interface 314 (e.g., an image sensor data user interface) in a three-dimensional environment 300 (e.g., in which a physical environment of the three-dimensional environment 300 is an operating room). The live camera feed user interface 314 includes live feed from camera 312. In FIG. 3A, computer system 101 presents table 308 and physical object 310 on table 308. Table 308 and physical object 310 are optionally physical objects of three-dimensional environment 300. In the illustrated example, a camera 312 (e.g., an image sensor) is disposed inside of object 310, and is capturing images inside of object 310. In some examples, object 310 is a body of a patient. In some examples, physical object 310 is representative of another type of physical object and/or is representative of one or more objects. In some examples, camera 312 is a laparoscopic camera, a stereoscopic camera, or another type of camera. In some examples, computer system 101 detects the live feed from camera 312 wirelessly and/or via a wired connection. As discussed above in the present discussion, although the following discussion is in the context of physical object 310 being a body of a patient, it should be noted that the physical object 310 is representative and could be different from a body of patient, such as a dummy model. In some examples, in response to user input (e.g., gaze input, input from a hand of the user, and/or voice input from the user, and/or another type of user input) requesting to move and/or resize the live camera feed user interface 314, the electronic device 101 moves and/or resizes the live camera feed user interface 314 in a direction and/or to a size associated with user input. In some examples, the live camera feed user interface 314 maintains its position relative to three-dimensional environment 300, and changes in position in response to user input requesting to move and/or resize the live camera feed user interface 314.

In FIG. 3A, while displaying live camera feed user interface 314, computer system 101 displays user interface elements 316a through 316d. These user interface elements are optionally selectable to cause the electronic device 101 to perform different operations. User interface element 316a is optionally selectable to initiate a process to display a widget dashboard user interface 330, such as described with reference to FIG. 3H. User interface element 316b is optionally selectable to initiate a process to capture and/or store an image or set of images detected by camera 312. User interface element 316c is optionally selectable to initiate a process for initiating a communication session between user 301 of computer system 101 and a user (e.g., a remote user who is not in the physical environment of computer system 101) of a different computer system. User interface element 316c is optionally selectable to initiate a process to display images that are optionally captured by camera 312, or by one or more different image sensors.

In FIG. 3B, while displaying the live camera feed user interface 314 of FIG. 3A, computer system 101 detects input from the user 301 (e.g., gaze 320a of the user 301, with or without another user input, input from a hand of the user 301 with or without another user input, voice input from the user 301 with or without another user input, and/or another type of input) directed at user interface element 316d. In response, computer system 101 presents three-dimensional environment 300 of FIG. 3C.

In FIG. 3C, computer system 101 concurrently displays live camera feed user interface 314, box 322a, 3D object 322b (e.g., a three-dimensional object) inside of box 322a, and user interface elements 324a through 324c. Box 322a is optionally a three-dimensional object having a transparent or semi-transparent fill, such that 3D object 322b is visible through box 322a, and 3D object 322b is optionally a 3D model (e.g., a model of an organ of the patient) for which the electronic device 101 can present different views. For example, computer system 101 can optionally rotate the 3D object 322b and/or display internal views corresponding to cross-sections of the 3D object (e.g., in response to input from user 301). In some examples, one or more dimensions of box 322a are modifiable (e.g., via user input from the user 301 of the electronic device 101 (e.g., voice input, gaze input, and/or input from a hand of the user detected by computer system 101)), and modifying the dimensions of box optionally results in display of different cross sections of 3D object 322b (e.g., different cross sections of 3D object 322b about the axis parallel to the axis of the box 322a that is modified). As such, computer system 101 permits a surgeon to view different cross sections of 3D object 322b during a surgical procedure, without need for the surgeon to rotate the surgeon's head to undesirable positions and/or without a ceasing of display of the live camera feed user interface 314 in the field of view of the user 301. In FIG. 3C, user interface elements 324a through 324c are optionally selectable to display different sets of images. For example, in the illustrated example of FIG. 3C, user interface element 324b is selected, which corresponds to display of 3D object 322b. If user interface element 324a is selected, computer system 101 would optionally replace display of box 322a and 3D object 322b with one or more images corresponding to scans of the patient. For example, user interface element 324a would optionally correspond to MRI scans that are scrubbable to specific MRI scans and/or to specific views of the MRI scan. If user interface element 324c is selected, computer system 101 would optionally replace display of box 322a and 3D object 322b with one or more images corresponding to images captured by camera 312. As such, computer system 101 permits a surgeon to view scans, captured images, and/or 3D object models during a surgical procedure, without need for the surgeon to rotate the surgeon's head to undesirable positions and/or without a ceasing of display of the live camera feed user interface 314 in the field of view of the user 301.

As shown in FIG. 3C, in top-down view 318c, live camera feed user interface 314 and box 322a are both facing the position of the user 301 (e.g., oriented toward a viewpoint of computer system 101) and are in the field of view of the user 301. Since these elements are located at different positions, these elements are angled relative to each other. That is, in the illustrated example, an angle a normal of live camera feed user interface 314 and box 322a is nonzero. As such, computer system 101 displays user interfaces at optimal positions for use by the user 301 during a surgical operation. Further, it should be noted that the user interfaces can be moved in response to user input. For example, in response to a voice input from the user 301 indicating a request to move live camera feed user interface 314 back in depth in the field of view of the user 301, toward the user 301 in the field of the user 301, up, down, or in another direction, the electronic device 101 optionally moves the live camera feed user interface 314 in accordance with the user input. It should also be noted that the electronic device 101 optionally stores in memory or storage a preferred location (e.g., user-preferred) of the live camera feed user interface 314 relative to a position in the operating room and/or relative to the patient, such that if the user 301 were to leave the operating room and then return to the operating room, the location of the live camera feed user interface 314 would optionally be maintained, such that when the user 301 returns to the operating room and uses computer system 101, the electronic device 101 would optionally display the live camera feed user interface 314 at the last position of the live camera feed user interface 314 in the room. Such features are likewise applicable to the electronic device 101 displaying the widget dashboard user interface 330 of FIG. 3H and/or other user interfaces and/or user interface elements described herein.

In FIG. 3D, while displaying live camera feed user interface 314, box 322a, 3D object 322b inside of box 322a, and user interface elements 324a through 324c, the electronic device 101 detects input from the user 301 (e.g., gaze 320b of the user 301, with or without another user input, input from a hand of the user 301 with or without another user input, voice input from the user 301 with or without another user input, and/or another type of input) directed at user interface element 316c. In response, computer system 101 presents three-dimensional environment 300 of FIG. 3E.

In FIG. 3E, computer system 101 concurrently displays live camera feed user interface 314, box 322a, 3D object 322b inside of box 322a, user interface elements 324a through 324c, and contact list user interface 326a. Contact list user interface 326a includes user interface elements corresponding to different contacts for which user 301 of computer system 101 can initiate a communication session. The communication session would optionally include video and/or audio feed between computer system 101 and a different computer system associated with a user in the contact list. In the illustrated example, each person in the contact list is represented with a name (e.g., “Dr. 1”) and an avatar (e.g., the circle icon above “Dr. 1”). In addition, computer system 101 displays respective selectable user interface elements for initiating a communication with the respective person. For example, in the illustrated example of FIG. 3E, immediately below “Dr. 1” is a user interface element that is selectable to call (e.g., via a phone call, video call, a ping, a message notification, etc.) the respective person indicating a request to for the respective person to join a communication session with user 301 of computer system 101.

While presenting three-dimensional environment 300 of FIG. 3E, the electronic device 101 detects alternative inputs from the user 301 (e.g., gaze 320c of the user 301 and gaze 320d of the user 301, with or without another user input, input from a hand of the user 301 with or without another user input, voice input from the user 301 with or without another user input, and/or another type of input). In the illustrated example, gaze 320c of the user 301 is directed at a user interface element 316c and corresponds to a request to initiate a call with “Dr. 1” and gaze 320d of the user 301 directed at user interface element 316a, as shown in FIG. 3F. The discussion that follows with reference to FIG. 3G is in response to gaze 320c of the user 301 and the discussion that follows with reference to FIG. 3H is in response to gaze 320d of the user 301. In response to the gaze 320c of the user 301 directed at a user interface element 316c corresponding to a request to initiate a call with “Dr. 1”, and optionally provided that a respective user of a respective computer system that corresponds to “Dr. 1” accepts the request, computer system 101 presents three-dimensional environment 300 of FIG. 3G. It should be noted that that if the respective user of the respective computer system that corresponds to “Dr. 1” does not accept the request, computer system 101 optionally initiates a process for user 301 to send a message (e.g., a voicemail message or a text message) to the respective user while maintaining presentation of three-dimensional environment 300 shown in FIG. 3F.

In FIG. 3G, computer system 101 concurrently displays live camera feed user interface 314, box 322a, 3D object 322b inside of box 322a, user interface elements 324a through 324c, and representation 326b of the user of the computer system that corresponds to “Dr. 1” and a user interface element corresponding to a request to end the call with the user of the computer system who corresponds to “Dr. 1”. In FIG. 3G, a communication session between user 301 of computer system 101 and the user of the computer system who corresponds to “Dr. 1” is active. As such, computer system 101 displays representation 326b, without displaying contact list user interface 326a. In some examples, representation 326b includes video feed (e.g., live video feed) of the user of the computer system who corresponds to “Dr. 1”. In some examples, the electronic device 101 transmits to the user of the computer system who corresponds to “Dr. 1” the three-dimensional environment 300 of FIG. 3G (e.g., without representation 326b and the user interface element corresponding to the request to end the call).

In response to the gaze 320d of the user 301 directed at a user interface element 316a in FIG. 3F, computer system 101 initiates a process to present three-dimensional environment 300 of FIG. 3H. It should be noted that any of the input described herein, such as gaze 320d is optionally alternatively a pinch gesture, and/or is a gaze input and a pinch of a user's hand.

In FIG. 3H, computer system 101 displays widget dashboard user interface 330. In some examples, computer system 101 visually transitions between the displaying the user interfaces of FIG. 3F and the widget dashboard user interface 330 in accordance with one or more animations. For example, in response to the gaze 320d of the user 301 directed at a user interface element 316a, computer system 101 optionally fades out from display contact list user interface 326a (e.g., reduces in visual prominence), box 322a, 3D object 322b, and user interface elements 324a-324c optionally at the same or different rates. Continuing with this example, in response to the gaze 320d of the user 301 directed at a user interface element 316a, computer system 101 optionally reduces a size (e.g., reduces actual and/or apparent dimension(s), such as horizontal and/or vertical dimensions) of live camera feed user interface 314 while maintaining display of live camera feed user interface 314 during the transition animation to the widget dashboard user interface 330. Continuing with this example, in response to the gaze 320d of the user 301 directed at a user interface element 316a, computer system 101 optionally visually moves user interface elements (e.g., widgets 328a through 328i) into the field of view of the user 301 to their respective positions illustrated in FIG. 3H, optionally while fading in (e.g., increasing a visual prominence of user interface elements 328a through 328i. For example, user interface elements (e.g., widgets 328a through 328i) are moved into the field of view of the user 301 to their respective positions illustrated in FIG. 3H based on their final position in the widget dashboard user interface 330. For example, computer system 101 optionally visually moves user interface elements (e.g., widgets 328a through 328c) optionally from left to right in the field of view of the user 301 to their respective final positions illustrated in FIG. 3H, user interface elements (e.g., widgets 328f and 328g) optionally upward in the field of view of the user 301 to their respective final positions illustrated in FIG. 3H, and user interface elements (e.g., widgets 328i and 328h) from right to left in the field of view of the user 301 to their respective final position illustrated in FIG. 3H.

Widget dashboard user interface 330 includes user interface elements (e.g., widgets 328a through 328i), in addition to live camera feed user interface 314a (which is optionally of a size (e.g., an actual or apparent size) that is smaller in one or more dimensions than live camera feed user interface 314 in FIG. 3F (e.g., the electronic device 101 reduced in size live camera feed user interface 314 of FIG. 3F to the size of live camera feed user interface 314a of FIG. 3H). In some examples, the depth of the placement of the live camera feed user interface 314a in FIG. 3H (e.g., relative to the viewpoint of the user 301 in FIG. 3H) is the same as the depth of the placement of the live camera feed user interface 314 in FIG. 3F (e.g., relative to the viewpoint of the user 301 in FIG. 3F). In some examples, the depth of the placement of the live camera feed user interface 314a in FIG. 3H is different from the depth of the placement of the live camera feed user interface 314 in FIG. 3F. As described herein, in some examples, physical object 310 is a patient's body and the user 301 is a medical provider, such as a surgeon. While interacting with the patient's body, the user 301 optionally desires to view one or more aspects of the patient and/or of the environment so as to maintain an optimal environment for the user 301 during operation on or interaction with the patient, who in the illustrated example, is in the view of display 120a. In the illustrated example, one or more widgets are illustrated in the context of a laparoscopic surgery (e.g., a laparoscopic surgical procedure).

Vitals widget 328a optionally includes indications of a heart rate of the user, oxygen saturation (SpO2), Non-invasive Blood Pressure (NIBP) data, and/or respiratory rate (RR). These indications are optionally updated in real-time and are based on data detected by equipment (e.g., electronic equipment) coupled to the patient. As such, computer system 101 optionally displays critical information for monitoring the patient at optimal positions in the field of view of the user 301 relative to the patient (e.g., relative to the object 310) and optionally without the need for looking at multiple physical displays in the physical environment of the user 301 to access such information, as the electronic device 101 displays such information for the user 301 at the optimal positions, which can be customized by the user 301 without the assistance of other surgical personnel. Further, computer system 101 present one or more notifications to the user 301, such as audio notifications, optionally in addition to visual notifications, in response to detecting that one or more vitals of the patient has changed (e.g., changed beyond a threshold).

Energy source widget 328b optionally includes indication(s) of energy sources. For example, during a surgery, an energy source for the specific surgery is optionally based on the type of surgery that is being performed and/or is to-be-performed. For example, during a laparoscopic surgery, an energy source may include monopolar electrosurgery or bipolar electrosurgery. As such, computer system 101 optionally displays energy source information based on the energy sources involved in the surgical procedure, which is useful for monitoring during a surgical operation.

Suction/irrigation widget 328c optionally includes indications of flow rates detected by suction and/or irrigation sensors, which may be useful for monitoring the patient's bodily behavior during the laparoscopic surgery. As such, computer system 101 is optionally in communication with various sensors and present such critical information to the user 301 at optimal, customized positions as described above.

Stereo disparity widget 328d optionally includes an indication of a level of stereo disparity. As discussed above, camera 312 is optionally configured to detect images in stereo and/or is optionally a stereoscopic camera. Stereo disparity widget 328d optionally includes a user interface element (e.g., a slider, a knob, a dial, a button, or another type of user interface element) that is selectable to set or change a level of stereo disparity. As such, widget dashboard user interface 330, via stereo disparity widget 328d, provides user 301 with the ability to perform various operations quickly with respect to other devices that are in communication (e.g., via a wired or wireless) connection with computer system 101, thus increasing a level of control and/or detail for the user 301, which likewise may reduce errors in surgical operations.

Passthrough dimming widget 328e optionally includes an indication of a level of passthrough dimming of the three-dimensional environment 300. As discussed above, in some examples, computer system 101 is optionally an optical see-through device in which display 120 is a transparent or translucent display through which portions of the physical environment may be directly viewed, display 120 may be included within a transparent lens and may overlap all or only a portion of the transparent lens, and/or computer system 101 is a video-passthrough device in which display 120 is an opaque display configured to display images of the physical environment captured by external image sensors 114b and 114c. Passthrough dimming widget 328c optionally includes a user interface element (e.g., a slider, a knob, a dial, a button, or another type of user interface element) that is selectable to set or change a level of passthrough dimming for the user 301. For example, the user 301 can change the level of passthrough dimming such that in the field of view of the 301, the electronic device 101 presents via passthrough the patient's body (e.g., object 310) without presenting passthrough of the physical environment different from the patient body. In this example, even if the operating room is well-lit, computer system 101 provides user 301 the ability to darken the visibility of the operating room in the field of view of the user 301 of computer system 101, without needing to change the level of physical light (e.g., emitted by one or more physical light sources, such as overhead lights, lamps, ambient light, etc.) inside of the physical environment. As such, widget dashboard user interface 330 provides user 301 with the ability to customize lighting settings for the user 301, optionally without changing a lighting setting of the operating room outside.

Scans widget 328f optionally includes display of one or more scans, such as magnetic resonance imaging (MRI) scans that correspond to the patient, such as described with reference to FIG. 3C. In response to detection of selection of scans widget 328f, the computer system optionally displays the one or more scans, in addition to a user interface element (e.g., a slider, a dial, a button, a knob, or another type of user interface element) for scrubbing through (e.g., zooming or viewing different captured scans) scans of the patient. As such, widget dashboard user interface 330 provides user 301 with the ability to access data corresponding to the patient, without the need of addition placements of physical tv screens in the operating room.

Captures widget 328g optionally includes display of one or more images captured by camera 312, such as described with reference to FIG. 3C. In some examples, the one or more images includes one or more images that include virtual annotations overlaid on the captured images, such as virtual annotations made by user 301 via computer system 101 or made by a remote user of a remote computer system, such as the remote user described with reference to FIG. 3G. In response to detection of selection of captures widget 328g, the electronic device 101 optionally displays a slider for scrubbing through scans of the patient. As such, widget dashboard user interface 330 provides user 301 with the ability to access data corresponding to the patient, without the need of addition placements of physical tv screens in the operating room.

Procedure summary widget 328h optionally includes textual display of a summary of the procedure that is to be performed on the patient, or that is being performed on the patient. The procedure summary widget 328h optionally identifies the type of the surgery, a diagnosis of the patient and/or the patient's condition (e.g., a pre-operative diagnosis that optionally resulted in the identification of the need for a surgical treatment), the surgeon (e.g., a name of the surgeon), a type of anesthesia that is to be used on the patient (e.g., general anesthesia, local anesthesia, etc.), a condition of the patient (e.g., critical condition, stable, unstable, etc.), and an identification of whether the patient has had previous surgeries. As such, widget dashboard user interface 330 provides user 301 with the ability to access data corresponding to the patient, without the need of addition placements of physical tv screens in the operating room.

Different users may use electronic device 101 at different times. In some examples, electronic device 101 stores customized widget dashboard user interfaces on a per user basis and presents the customized widget dashboard user interfaces in accordance with the specific user of the electronic device. For example, in accordance with a determination that the user of the electronic device is a first user, widgets in the widget dashboard user interface may include a first set of widgets, such as the widgets in widget dashboard user interface 330 in FIG. 3H, optionally because the first user requested said widgets to be in widget dashboard user interface 330 in FIG. 3H. Continuing with this example, in accordance with a determination that the user of the electronic device is a second user, different from the first user, widgets in the widget dashboard user interface may include a second set of widgets that is different from the first set of widgets, optionally because the second user requested said widgets to be in the widget dashboard user interface. In some examples, the first set of widgets includes a first amount of widgets, and the second set of widgets includes a second amount of widgets that is different from the first amount of widgets. In some examples, the first set of widgets are selectable to view first data and the second of widgets are selectable to view second data different from the first data. In some examples, the first set of widgets is equal in amount to the second set of widgets. In some examples, the first set of widgets is equal in amount to the second set of widgets, the first set of widgets includes the same widgets as those in the second set of widgets, and the first set of widgets are arranged in a first arrangement on the widget dashboard user interface and the second set of widgets is arranged in a second arrangement on the widget dashboard user interface that is different from the first arrangement. As such, the electronic device optionally presents different customized widget dashboard user interfaces to different users in accordance with differences in customizations made by or for the different users.

In some examples, the electronic device 101 may detect and respond to input for customizing a widget dashboard user interface. For example, while displaying the widget dashboard user interface 330 in FIG. 3H, the electronic device 101 may detect a request to add an additional widget. For example, the electronic device 101 may detect a voice input from the user or another input corresponding to a request to add the additional widget to the widget dashboard user interface 330 in FIG. 3H. In response, the electronic device 101 may display the widget dashboard user interface 330 of FIG. 3H including the additional widget. Further, as another example, while displaying the widget dashboard user interface 330 in FIG. 3H, the electronic device 101 may detect a request to remove a respective widget from the dashboard user interface. For example, the electronic device 101 may detect a request from the user to remove suction/irrigation widget 328c from widget dashboard user interface 330. In response, the electronic device 101 may displaying the widget dashboard user interface 330 without the respective widget (e.g., without suction/irrigation widget 328c).

In addition, the electronic device 101 may detect and respond to input for rearranging widgets of the widget dashboard user interface 330. For example, the electronic device 101 may detect a request to move procedure summary widget 328h to the location of passthrough dimming widget 328e. In response, the electronic device 101 may update display of widget dashboard user interface 330 to have procedure summary widget 328h at the location where passthrough dimming widget 328e appears in FIG. 3H.

It should be noted that the examples with reference to FIGS. 3A-3H with the computer system detecting a gaze of the user is additionally and/or alternatively applicable to the computer system detecting a voice input from the user, with or without gaze, and/or with or without detection of other inputs.

FIG. 3I is a flow diagram illustrating a method 350 for displaying a widget dashboard user interface according to some examples of the disclosure. It is understood that method 350 is an example and that more, fewer, or different operations can be performed in the same or in a different order. Additionally, the operations in method 350 described below are, optionally, implemented by running one or more functional modules in an information processing apparatus such as general-purpose processors (e.g., as described with respect to FIG. 2) or application specific chips, and/or by other components of FIG. 2.

Therefore, according to the above, some examples of the disclosure are directed to a method (e.g., method 350 of FIG. 3I) comprising, at a computer system in communication with one or more displays and one or more input devices, including a camera and one or more sensors, different from the camera, while a physical object is visible via the one or more displays, displaying (352), via the one or more displays, a widget dashboard user interface, including a first widget including live camera feed from the camera, and one or more second widgets including one or more indications of the physical object. In some examples, the one or more indications of the physical object is based on data detected by the one or more sensors.

Additionally or alternatively, in some examples, the first widget and/or the one or more second widgets updates in real-time based on updates in data from the one or more sensors.

Additionally or alternatively, in some examples, the physical object is a body of a patient, and a user of the computer system is a surgeon, and the method is performed while the surgeon is performing a surgical operation on the body of the patient.

Additionally or alternatively, in some examples, the live camera feed from the camera is of a first size in the field of view of the user, and the method includes detecting, via the one or more input devices, a first user input directed at the first widget, and in response to detecting the first user input directed at the first widget, ceasing display of the one or more second widgets, and displaying, via the one or more displays, the live camera feed from the camera having a second size greater than the first size.

Additionally or alternatively, in some examples, method 350 includes in response to detecting the first user input directed at the first widget, displaying, via the one or more displays, one or more user interface elements, including a first user interface element selectable to initiate a first process, the first process including displaying the widget dashboard user interface, a second user interface element selectable to initiate a second process, the second process including capturing one or more images from the camera, a third user interface element selectable to initiate a third process, the third process including initiating a communication session with a second computer system, and a fourth user interface element selectable to initiate a fourth process, the fourth process including displaying captured data from the camera, a model of a three-dimensional object, and/or captured data from an image sensor different from the camera. Additionally or alternatively, in some examples, method 350 includes detecting, via the one or more input devices, selection of the fourth user interface element, and in response to detecting selection of the fourth user interface element, initiating the fourth process, including concurrently displaying, via the one or more displays the live camera feed from the camera having the second size, and the model of the three-dimensional object. Additionally or alternatively, in some examples, the first user interface element is maintained in display in response to the detection of the selection of the fourth user interface element, and method 350 includes detecting, via the one or more input devices, selection of the first user interface element, and in response to detecting selection of the first user interface element, initiating the first process, including ceasing display of the model of the three-dimensional object and displaying, via the one or more displays, the widget dashboard user interface. Additionally or alternatively, in some examples, initiating the first process includes reducing, from the second size to the first size, the live camera feed from the camera and animating movement of the one or more second widgets to respective locations in the widget dashboard user interface.

Additionally or alternatively, in some examples, in accordance with a determination that a user of the computer system is a first user, the one or more second widgets include a first set of widgets and in accordance with a determination that the user of the computer system is a second user, different from the first user, the one or more second widgets include a second set of widgets. Additionally or alternatively, in some examples, the first set of widgets is the second set of widgets. Additionally or alternatively, in some examples, the first set of widgets is different from the second set of widgets.

Additionally or alternatively, in some examples, in accordance with a determination that the user of the computer system is the first user, widgets of the widget dashboard user interface have a first arrangement in the widget dashboard user interface and in accordance with a determination that the user of the computer system is a second user, different from the first user, widgets of the widget dashboard user interface have a second arrangement in the widget dashboard user interface. Additionally or alternatively, in some examples, the first arrangement is the second arrangement in the widget dashboard user interface. Additionally or alternatively, in some examples, the first arrangement in the widget dashboard user interface is different from the second arrangement in the widget dashboard user interface.

Additionally or alternatively, in some examples, method 350 comprises while displaying the widget dashboard user interface including the first widget and the one or more second widgets, detecting, via the one or more input devices, a request to add an additional widget and in response to detecting the request, displaying, via the one or more displays, the widget dashboard user interface including the first widget, the one or more second widgets, and the additional widget. Additionally or alternatively, in some examples, method 350 comprises while displaying the widget dashboard user interface including the first widget and the one or more second widgets, detecting, via the one or more input devices, a request to remove a respective widget from the dashboard user interface and in response to detecting the request to remove the respective widget from the dashboard user interface, displaying the widget dashboard user interface without the respective widget.

Additionally or alternatively, in some examples, method 350 is performed in the recited order of the method.

Additionally or alternatively, in some examples, the one or more displays includes a head-mounted display.

Attention is now directed towards examples of an electronic device displaying a representation of a physical tool for indicating a location of the physical tool relative to a location associated with video feed, and toward examples of an electronic device displaying indications of proximities of physical tools relative to one or more surfaces of a physical object.

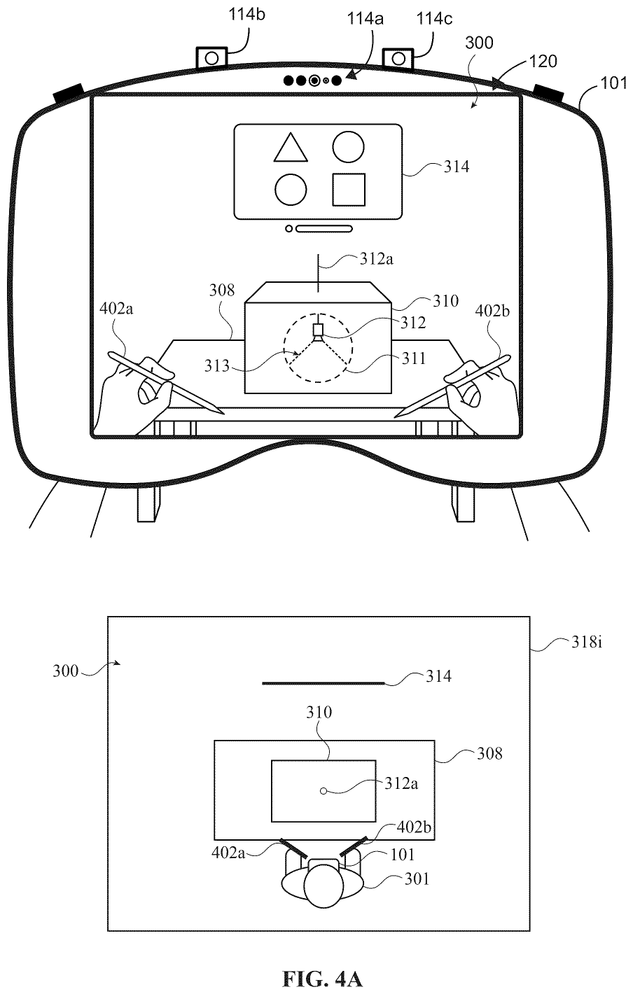

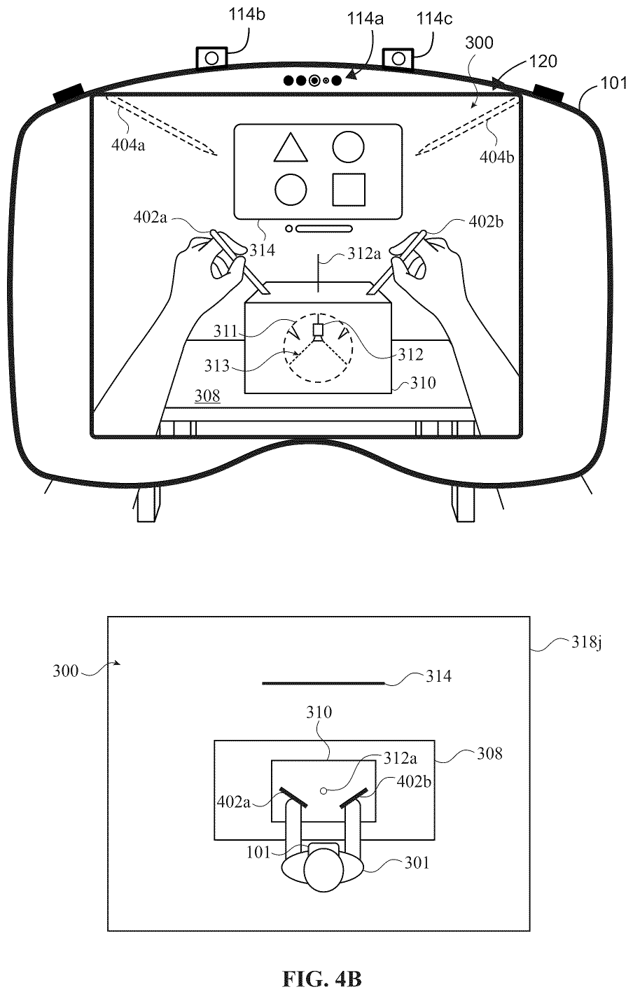

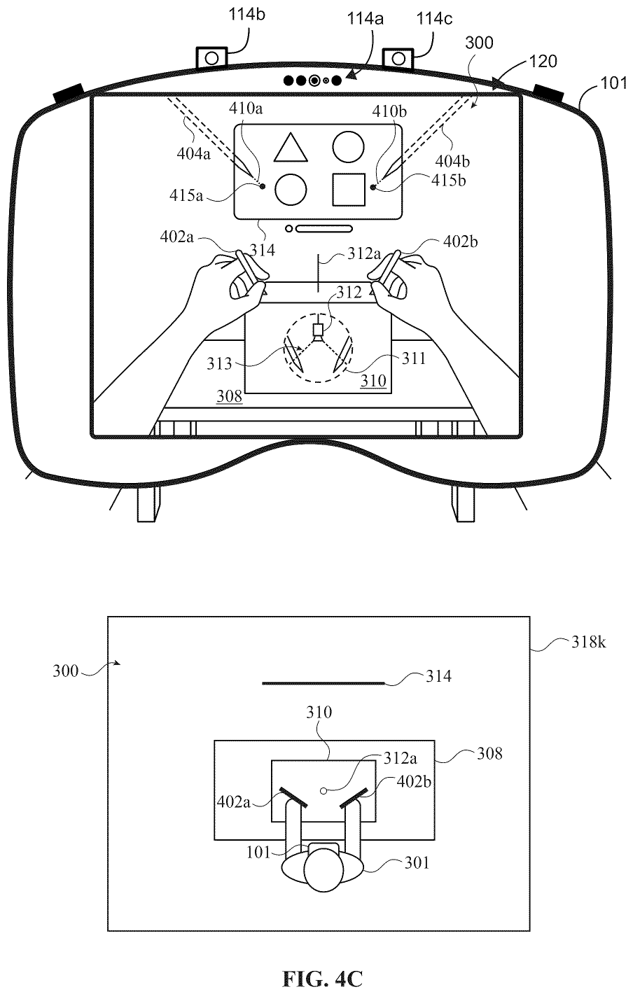

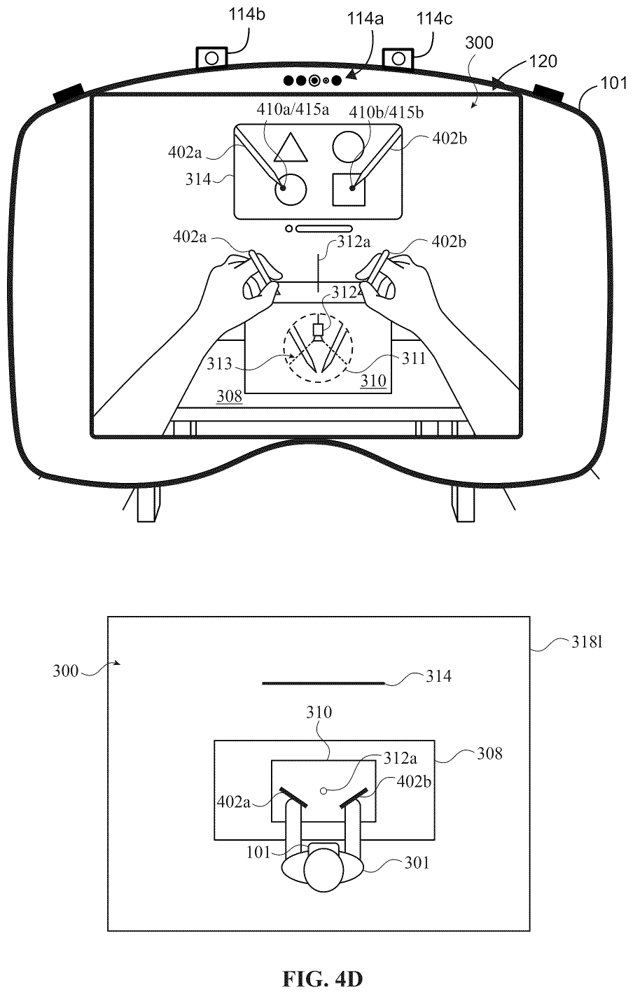

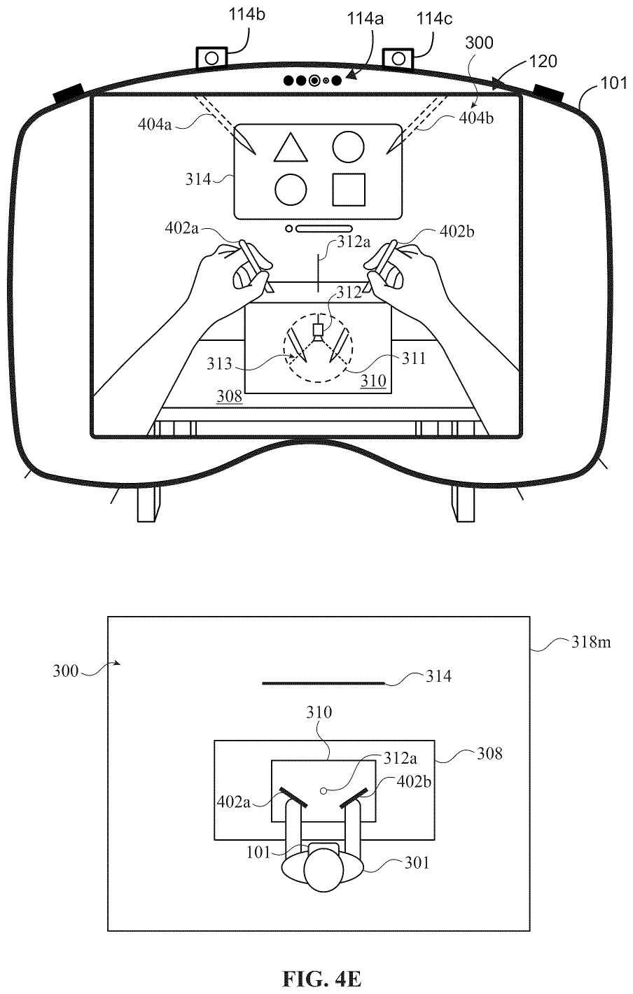

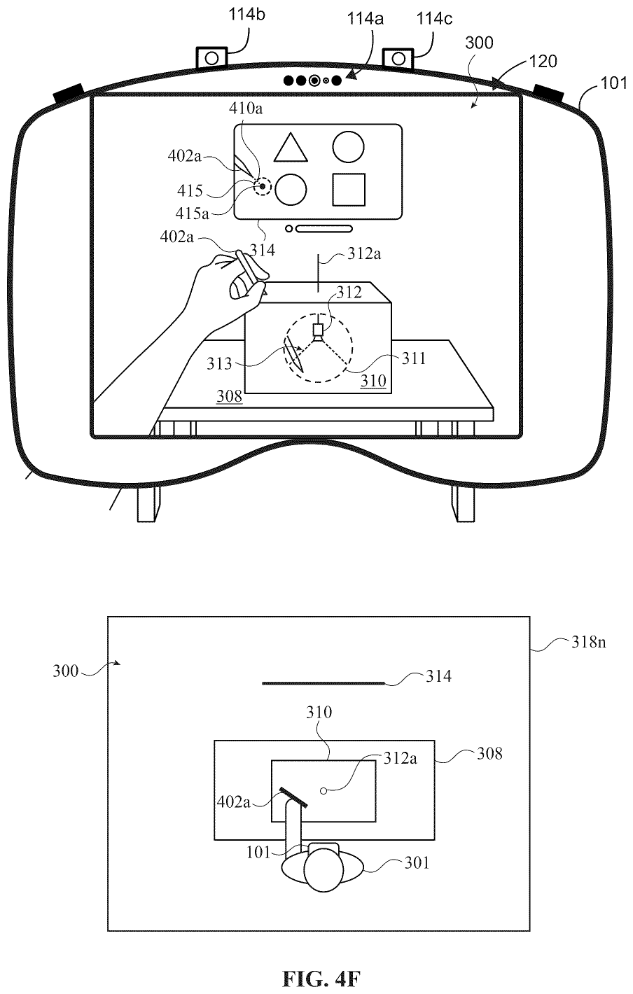

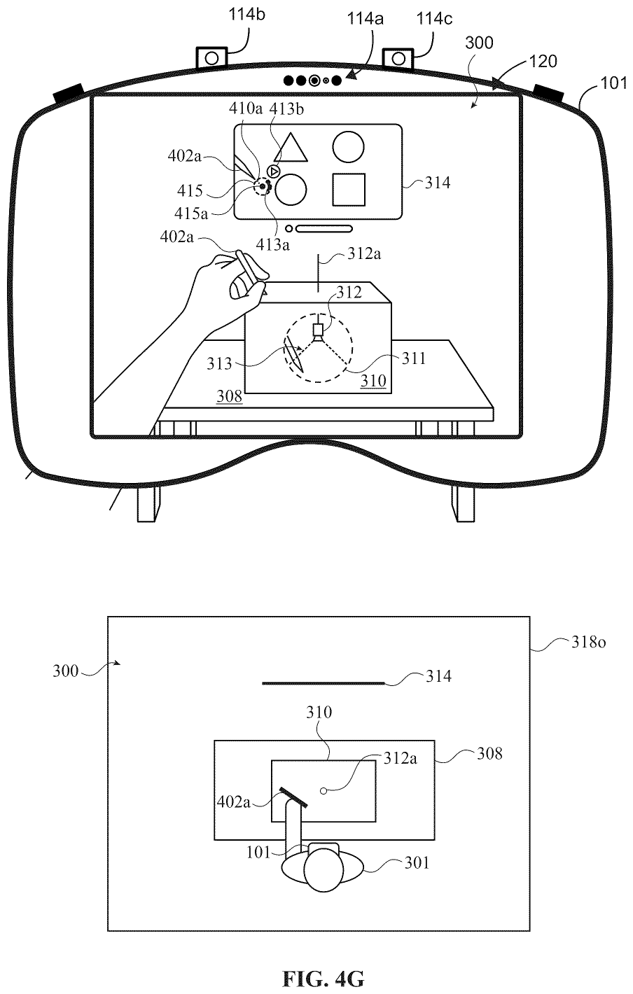

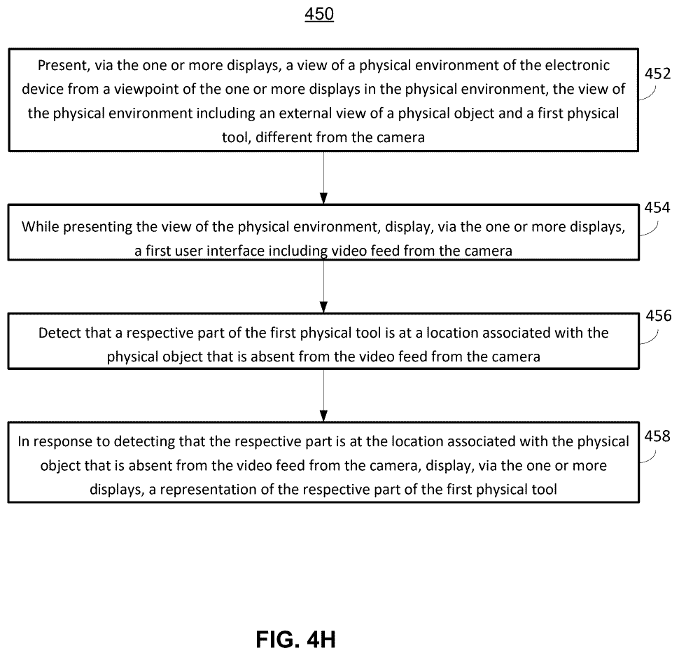

FIGS. 4A-4G illustrate examples of an electronic device displaying a representation of a physical tool in accordance with satisfaction of criteria, according to some examples of the disclosure.

For the purpose of illustration, FIGS. 4A-4G include respective top-down views 318i-3180 of the three-dimensional environment 300 that indicate the positions of various objects (e.g., real and/or virtual objects) in the three-dimensional environment 300 in a horizontal dimension and a depth dimension. The top-down view of the three-dimensional environment 300 further includes an indication of the viewpoint of the user 301 of the electronic device 101. For example, in FIG. 4A, the electronic device 101 displays the view of the three-dimensional environment 300 visible through the display 120 from the viewpoint of the user 301 illustrated in the top-down view 318i of the three-dimensional environment 300.

In FIG. 4A, electronic device 101 is displaying live camera feed user interface 314 while physical tools 402a/402b are not yet in the physical object 310. As an example, in FIG. 4A, physical tools 402a/402b are surgical tools, physical object 310 is a body of a (e.g., human) patient, camera 312 is a laparoscopic camera whose camera feed (e.g., detected image data from inside the physical object 310) is being displayed in live camera feed user interface 314, and the surgical tools are outside of the body while camera 312 is inside of the body. In some examples, the electronic device 101 detects the locations of the physical tools 402a/402b relative to the physical object 310 via external image sensors 114b/114c of the electronic device 101 that face the physical object 310, such as image sensor(s) 206 including outward facing sensors. For example, the electronic device 101 may determine that physical tools 402a/402b are not yet in the physical object 310 because the electronic device 101 has detected image data of the physical tools 402a/402b and of the physical object 310 and determined that these are not in contact and/or do not overlap with each other.

Further, the electronic device 101 optionally detects the pose (e.g., position and orientation) of the camera 312 relative to the physical object 310, in addition to detecting the image data that the camera 312 is capturing inside the physical object 310. For example, the electronic device 101 may detect the pose of the camera 312 via external image sensors 114b/114c of the electronic device 101 that face the physical object 310, such as image sensor(s) 206 including outward facing sensors. For example, the electronic device 101 may detect the pose (e.g., position and orientation) of the camera 312 by detecting the pose (e.g., position and orientation) of camera part 312a. In FIG. 4A, camera part 312a may be in the field of view of the external image sensors 114b/114c of electronic device 101, and the amount of camera part 312a that is in the view (e.g., in the viewpoint of the user 301) and the angular orientation of the camera part 312a may indicate the pose of the camera 312, which in FIG. 4A, is in the physical object 310 and may not be seen from the viewpoint of the user 301 of the electronic device 101. For example, camera part 312a is optionally a portion of a surgical laparoscopic camera that is being held by a person or a structure, and its pose may indicate a pose of the camera 312 that is in the physical object 310. In some examples, the angular orientation of the camera part 312a is based on the angle between the camera part 312a and the force of gravity and/or the angle between the camera part 312a and the electronic device 101 (e.g., a vector extending from the viewpoint of the electronic device 101). Note that movement of the camera part 312a may result in movement of the camera 312 inside the physical object 310, and that moving the camera 312 via user input may include user input (e.g., user contact with the camera part 312a).

Note that in FIGS. 4A-4E, the inside of physical object 310 is visible to the user 301 of the electronic device 101 solely via live camera feed user interface 314 which shows the feed from camera 312, which is inside the physical object 310. That is, visibility of the inside of physical object 310 is provided via live camera feed user interface 314 which streams the feed from camera 312, and the cross section 311 is provided for illustration of the field of view 313 of the camera 312 and of the positioning of physical tools 402a/402b relative to the field of view 313 of the camera 312 in the applicable figure.

From FIG. 4A to 4B, the electronic device 101 detects that physical tools 402a/402b are in the physical object 310. For example, the electronic device 101 optionally detects that the physical tools 402a/402b have been moved into the physical object 310 via image sensor(s) 206 that detect the positions of the physical tools 402a/402b. Further, in FIG. 4B, the electronic device 101 optionally detects that though the physical tools 402a/402b have been moved into the physical object 310, physical tools 402a/402b are not in the field of view 313 of the camera 312. That is, in FIG. 4B, though the physical tools 402a/402b are inside the physical object 310, no portion of the physical tools 402a/402b are in the field of view 313 of the camera 312. If a portion of the physical tools 402a/402b were in the view of the camera 312, the portion would be displayed in the live camera feed user interface 314 because the live camera feed user interface shows image data that is in the field of view 313 of the camera 312. However, in FIG. 4B, no portion of the physical tools 402a/402b is in the view of the camera 312, so the illustrated example does not include the physical tools 402a/402b in the live camera feed user interface 314. In some examples, in response to detecting that the physical tools 402a/402b have been moved into the physical object 310 but are not yet in the field of view 313 of the camera 312, the electronic device 101 displays representations 404a/404b of respective portions of the physical tools 402a/402b, such as shown in FIG. 4B.

In the illustrated example of FIG. 4B, representations 404a/404b each include a tip portion and a body portion. In FIG. 4B, the representations 404a/404b include these portions because they are not in the field of view 313 of the camera 312 (e.g., as indicated by live camera feed user interface 314) though the physical tools 402a/402b are inside of the physical object 310, as described above. In the illustrated example of FIG. 4B, representations 404a/404b are displayed with a respective spatial arrangement relative to the live camera feed user interface 314 (e.g., at locations that are relative to the live camera feed user interface 314). The electronic device 101 optionally displays representations 404a/404b at their illustrated locations based on a spatial arrangement of physical tools 402a/402b relative to the field of view 313 of the camera 312 in the physical object 310 (e.g., to indicate the locations of the physical tools 402a/402b relative to the field of view 313 of the camera 312). For example, in FIG. 4B, the locations of the representations 404a/404b are to the left and right of the live camera feed user interface 314, respectively, and correspond to the locations of the physical tools 402a/402b being to the left and right of the camera 312 in the physical object 310 relative to the viewpoint of the user (e.g., relative to the electronic device 101), respectively. Further, in FIG. 4B, a first separation distance is between representation 404a and live camera feed user interface 314 and a second separation distance is between representation 404b and live camera feed user interface 314. In some examples, the first separation distance is based on the distance between physical tool 402a and the field of view 313 of the camera 312 (e.g., a position within the field of view 313). In some examples, the second separation distance is based on the distance between physical tool 402b and the field of view 313 of the camera 312 (e.g., a position within the field of view 313). As such, the electronic device 101 optionally displays the representations 404a/404b at locations that correspond to locations of the physical tools 402a/402b relative to the field of view 313 of the camera 312 from the viewpoint of the user. Further, in some examples, the electronic device 101 updates the locations of the representations 404a/404b in accordance with detected movement of the physical tools 402a/402b while physical tools 402a/402b are not in the view of the camera 312 but are inside of the physic object 310. In this way, the electronic device 101 displays a visual animation of movement of the representations 404a/404b that confirms that parts of the physical tools 402a/402b that are not in the field of view 313 of the camera 312 are being moved within the physical object 310. Note that the electronic device 101 may determine a pose of camera 312, and a pose of physical tool 402a (e.g., a pose of the tip of physical tool 402a) and a pose of physical tool 402b (e.g., a pose of the tip of physical tool 402b) relative to the field of view 313 of the camera 312 (e.g., relative to a position within the field of view 313 of the camera 312) using image data captured by external image sensors 114b/114c of the electronic device 101. For example, the electronic device 101 may detect, via external sensors 114b/114c, image data that includes camera part 312a to determine the pose of camera 312 and may detect, via external sensors 114b/114c, image data that includes portions of physical tools 402a/402b that are outside of the physical object 310 to determine poses of the physical tools 402a/402b that are inside of the physical object 310. For example, the electronic device 101 may already have access to data from which a relationship between a pose of camera part 312a and camera 312 may be deduced. Similarly, the electronic device 101 may already have access to data from which a relationship between a pose of a first portion of physical tool 402a (e.g., a portion that is inside the physical object 310) may be deduced based on a knowledge of a pose of a second portion of physical tool 402a (e.g., a portion that is outside the physical object 310). Likewise, the electronic device 101 may already have access to data from which a relationship between a pose of a first portion of physical tool 402b may be deduced based on a knowledge of a pose of a second portion of physical tool 402b. For example, the spatial arrangement between the physical tools 402a/402b and the camera 312 may be determined by the electronic device 101 detecting, via external image sensors 114b/114c, the poses of physical tools 402a/402b (e.g., the portions of physical tools 402a/402b that are in the field of view 313 of the camera 312) and the pose of camera part 312a and determining the spatial arrangement based on the detected image data.

From FIG. 4B to FIG. 4C, the physical tools 402a/402b are moved to respective locations that are in the field of view 313 of the camera 312. That is, portions of physical tools 402a/402b are in the field of view 313 of the camera 312 in FIG. 4C. In response, the electronic device 101 accordingly updates display of the live camera feed user interface 314 to include the respective portions of physical tools 402a/402b that are now in the field of view 313 of the camera 312, ceases display of at least a portion of the representations 404a/404b that corresponded to the parts of the physical tools 402a/402b that are now in the field of view 313 of the camera 312 (e.g., reduces the lengths of the representations 404a/404b), and moves toward the live camera feed user interface 314 the remaining portions of the representations 404a/404b that correspond to parts of the physical tools 402a/402b that still are not in the field of view 313 of the camera 312 to indicate that movement of the physical tools 402a/402b toward the view of camera 312 in the physical object 312 has occurred. From FIG. 4B to FIG. 4C, the tips and portions of the bodies of physical tools 402a/402b have been moved into the field of view 313 of the camera 312 and the electronic device 101 has ceased displaying parts of the representations 404a/404b that corresponded to tips and portions of physical tools 402a/402b that are now in the field of view 313 of the camera 312. In FIG. 4C, the tips and portions of the bodies of physical tools 402a/402b that are inside the field of view 313 of the camera 312 are being shown in live camera feed user interface 314, and said parts are not being represented in representations 404a/404b in FIG. 4C. Further, since less of the physical tool 402a/402b are outside of the field of view 313 of the camera 312 in FIG. 4C, the electronic device 101 has reduced a size of representations 404a/404b from FIG. 4B to FIG. 4C. For example, a longitudinal length of representations 404a/404b in FIG. 4C is less than a longitudinal length of representations 404a/404b in FIG. 4B. Ceasing display of at least the portion of the representations 404a/404b that corresponded to the parts of the physical tools 402a/402b that are now in the field of view 313 of the camera 312 provides a confirmation that the parts of the physical tools 402a/402b that corresponded to at least the portion of the representations 404a/404b are now in the field of view 313 of the camera 312.

In some examples, the electronic device 101 displays indications of proximities of physical tools relative to one or more surfaces of a physical object. In some examples, the electronic device 101 displays pointers 410a/410b, such as shown in FIG. 4C. For example, in FIG. 4C, the electronic device 101 displays, in the live camera feed user interface 314, the pointer 410a extending between the tip of the physical tool 402a and a part of an internal surface of physical object 310 to which the tip points. In some examples, the pointer 410a includes a portion (e.g., a visual portion) extending from the tip to a point on the surface of the physical object 310 to which the tip of physical tool 402a is pointing, and includes a visual indication 415a projected on the surface of the physical object 310 to which the tip of physical tool 402a is pointing (e.g., based on a determined vector extending from the tip of the physical tool 402a to the surface of the physical object 310), as shown in live camera feed user interface 314 in FIG. 4C. In some examples, the greater the distance between the tip of the physical tool 402a and the surface of the physical object 310 to which the tip of the physical tool 402a is pointing, the greater in size the visual indication 415a of the pointer 410a that is projected on the surface to which the tip of the physical tool 402a is pointing. In some examples, if the distance between the tip of the physical tool 402a and the surface of the physical object 310 to which the tip of physical tool 402a is pointing is a first distance, the pointer 410a (e.g., the portion and/or the visual indication 415a) is a first length in the live camera feed user interface 314, and if the distance between the tip of the physical tool 402a and the surface of the physical object 310 to which the tip of physical tool 402a is pointing is a second distance, different than the first distance, the pointer 410a is a second length that is different from the first length. As such, in some examples, pointer 410a indicates a distance between the tip of the physical tool 402a and a surface (e.g., internal surface) of the physical object 310 to which the tip of physical tool 402a is pointing. In some examples, a length of the pointer 410a in the live camera feed user interface 314 is based on the pose of the physical tool 402a relative to the field of view 313 of the camera 312. For example, if the pose of the physical tool 402a is a first pose that is more parallel and coincident to a line extending from the camera 312 to the portion of the physical object 310 that the tip of the physical tool 402a points toward than a second pose of the physical tool 402a, then the pointer 410a may be a first length, and if the pose is the second pose, then the pointer 410a pointer may be a second length that is different from (e.g., less than) the first length. In some examples, the electronic device 101 moves and/or updates display of the pointer 410a in accordance with a change of the pose (e.g., position and/or orientation) of the physical tool 402a. In some examples, the electronic device 101 changes a length of the pointer 410a based on changes in distance between the tip of the physical tool 402a and the surface of the physical object 310 to which the tip is pointing.

Further, in FIG. 4C, the electronic device 101 displays, in the live camera feed user interface 314, the pointer 410b extending between the tip of the physical tool 402b and a part of an internal surface of physical object 310 to which the tip points. In some examples, the pointer 410b includes a portion (e.g., a visual portion) extending from the tip to a point on the surface of the physical object 310 to which the tip of physical tool 402b is pointing, and includes a visual indication 415b projected on the surface of the physical object 310 to which the tip of physical tool 402b is pointing (e.g., based on a determined vector extending from the tip of the physical tool 402b to the surface of the physical object 310), as shown in live camera feed user interface 314 in FIG. 4C. In some examples, the greater the distance between the tip of the physical tool 402b and the surface of the physical object 310 to which the tip of the physical tool 402b is pointing, the greater in size the visual indication 415b of the pointer 410b that is projected on the surface to which the tip of the physical tool 402b is pointing. In some examples, if the distance between the tip of the physical tool 402b and the surface of the physical object 310 to which the tip of physical tool 402b is pointing is a first distance, the pointer 410b (e.g., the portion and/or the visual indication 415b) is a first length in the live camera feed user interface 314, and if the distance between the tip of the physical tool 402b and the surface of the physical object 310 to which the tip of physical tool 402b is pointing is a second distance, different than the first distance, the pointer 410b is a second length that is different from the first length. As such, in some examples, pointer 410b indicates a distance between the tip of the physical tool 402b and a surface (e.g., internal surface) of the physical object 310 to which the tip of physical tool 402b is pointing. In some examples, a length of the pointer 410b in the live camera feed user interface 314 is based on the pose of the physical tool 402b relative to the field of view 313 of the camera 312. For example, if the pose of the physical tool 402b is a first pose that is more parallel and coincident to a line extending from the camera 312 to the portion of the physical object 310 that the tip of the physical tool 402b points toward than a second pose of the physical tool 402b, then the pointer 410b may be a first length, and if the pose is the second pose, then the pointer 410b pointer may be a second length that is different from (e.g., less than) the first length. In some examples, the electronic device 101 moves and/or updates display of the pointer 410a in accordance with a change of the pose (e.g., position and/or orientation) of the physical tool 402b. In some examples, the electronic device 101 changes a length of the pointer 410b based on changes in distance between the tip of the physical tool 402b and the surface of the physical object 310 to which the tip is pointing.

From FIG. 4C to FIG. 4D, the physical tools 402a/402b are moved further toward respective locations in the field of view 313 of the camera 312. In FIG. 4D, a greater amount of the physical tool 402a and a greater amount of the physical tool 402b are in the field of view 313 of the camera 312 than in FIG. 4C. In response, the electronic device 101 ceases display of the representations 404a/404b, as shown in FIG. 4D. Ceasing display of the representations 404a/404b may confirm that the physical tools 402a/402b (e.g., that the greater amount of the physical tools 402a/402b) are now in the field of view 313 of the camera 312. In some examples, the representations 404a/404b cease to be displayed in response to detecting that a certain amount (e.g., a threshold portion, such as 50, 55, 60, 65, 70, 80, etc. %) of the physical tools 402a/402b are in the field of view 313 of the camera 312. In some examples, the representations 404a/404b cease to be displayed after a threshold amount of time (e.g., 4, 5, 10, 15, 30 s, or another amount of time) has passed since the electronic device 101 has detected movement of the physical tools 402a/402b toward or away from the field of view 313 of the camera 312. Thus, in FIGS. 4A through 4C, the electronic device 101 displays indications of the relative locations of the physical tools 402a/402b even when said locations were not in the field of view 313 of the camera 312 (and/or were not in the viewpoint of the user). Such features enhance for electronic-based instrument guidance.

In addition, in FIG. 4D, the pointers 410a/410b have changed in visual appearances (e.g., length, size, etc.) compared with their appearances in FIG. 4C. For example, in FIG. 4C, the pointers 410a/410b each include a portion extending between the tip of the physical tool and the surface to which the tip points and include a visual projection on the surface to which the tip points, while in FIG. 4D, the pointers 410a/410b solely include the visual projection on the surfaces to which the tips point (e.g., the visual indications 413a/413b). In some examples, the electronic device changes the visual appearances of the pointers 410a/410b as described because the position each tip of physical tool 402a/402b is contacting an internal surface of physical object 310.

In some examples, while the physical tools 402a/402b are in field of view 313 of the camera 312, and while the electronic device 101 is not displaying representations 404a/404b, such as in FIG. 4D, the electronic device 101 detects movement of physical tools 402a/402b to locations that are outside of the field of view of the camera 312. In response, the electronic device 101 displays (e.g., redisplays) representations 404a/404b of respective portions of the physical tools 402a/402b corresponding to the portions of the physical tools 402a/402b that are no longer in the view of the camera 312, such as from FIG. 4D to FIG. 4E.

In particular, from FIG. 4D to FIG. 4E, portions of the physical tools 402a/402b that were in the field of view 313 in FIG. 4D have been moved to outside of the field of view 313, while other portions of the physical tools 402a/402b that were in the field of view 313 in FIG. 4D are still in the field of view 313 in FIG. 4E. In response to detecting that portions of the physical tools 402a/402b that were in the field of view 313 in FIG. 4D having been moved to outside of the field of view 313, the electronic device 101 initiates display of representations 404a/404b to indicate the portions of physical tools 402a/402b that are no longer in the field of view 313 of the camera 312. In this way, the electronic device 101 displays a visual animation that confirms to the user 301 that some portions of physical tools 402a/402b are detected as being moved to outside of the field of view 313 of camera 312 even though other portions of the physical tools 402a/402b are still in the field of view 313 of the camera 312. For example, in the illustration of FIG. 4E, representations 404a/404b do not include respective portions that represent tips of the physical tools 402/a/402b because the physical tips of the physical tools 402a/402b are still in the field of view 313 of the camera 312 in FIG. 4E. In some examples, the electronic device 101 displays representations 404a/404b in accordance with a determination that physical tools 402a/402b are being moved to outside of the field of view 313 of the camera 312. Additionally or alternatively, in some examples, the electronic device 101 displays representations 404a/404b in accordance with a determination that physical tools 402a/402b are being moved to inside of the field of view 313 of the camera 312.

In some examples, in FIG. 4E, if further movement away from the field of view 313 of the camera 312 is detected, the electronic device 101 would correspondingly increase the lengths of the representations 404a/404b in accordance with the movement (e.g., until the physical tools 402a/402b are outside of the field of view 313 of the camera 312, at which point the representations 404a/404b would optionally have a maximum length such as the length of representations 404a/404b in FIG. 4B, and would optionally include representations of the tips of the physical tools 402a/402b). In some examples, after increasing the length of the representations 404a/404b while detecting movement of the physical tools to outside of the field of view 313 of the camera 312, the electronic device 101 ceases display of the representations 404a/404b. As such, the electronic device 101 optionally assists and guides its user when it detects movement of the physical tools towards or away from being within the field of view 313 of the camera 312. Such features enhance physical tool placement even when portions of the physical tool are not visible to the user.

Note that the electronic device may display and/or cease display of representation 404a of physical tool 402a independently of display and/or of ceasing display of representation 404b of physical tool 402b. Also, note that the number of physical tools illustrated in the figures is representative, that fewer or more physical tools may be present, and that more or fewer representations of the tools may be displayed based on the detected number of physical tools.

FIGS. 4F and 4G illustrate an example of the electronic device 101 displaying pointer 410a and a visual indication 415 projected on the surface of the physical object 310 about the visual indication 415a of the pointer 410a. In some examples, the visual indication 415 visually notifies the user 301 of an area (e.g., region, and/or portion) of the physical object 310 that would be affected by the physical tool 402a were the physical tool 402a within a threshold distance of the area (e.g., region and/or portion). For example, physical tool 402a may be a cauterization instrument that is heated and a surface of the physical object 310 that is within a threshold distance of the area may be affected (e.g., burned, dissolved, removed, etc.) by the physical tool 402a. In FIG. 4F, the surface of the physical object 310 is not within the threshold distance of the area and in FIG. 4G, a surface of the physical object 310 is within the threshold distance of the area. However, the surface of the physical object 310 that is within the threshold distance of the area is not a surface to which the physical tool 402a is supposed to affect (e.g., the cauterization tool is not supposed to affect the surface in the illustrated example, as that surface is not the surface to which cauterization is desired in the operation that involves use of the cauterization), so the electronic device 101 displays additional indications 413a/413b that provide a warning that the surface of the physical object 310 that is covered by the visual indication 415 is within the threshold distance of the area of the physical object 310. In some examples, a visual prominence (e.g., a brightness, a contrast, a shade of color, etc.) of the indication 413a (and/or of the indication 413b) is a function of distance between respective points or areas of the surface of the physical object 310 that is covered by the visual indication 415. In some examples, the smaller the distance, the greater the visual prominence of the indication 413a. In some examples, the indication 413a includes a first part and a second part, and the electronic device 101 concurrently displays the first part of the indication 413a with a first visual prominence and the second part of the indication 413a with a second visual prominence that is different from (e.g., more than or less than) the first visual prominence because the distance between the first part and the physical tool 402a is different from the distance between the second part and the physical tool 402a. In some examples, were the distance between the first part and the physical tool 402a the same as the distance between the second part and the physical tool 402a, the electronic device 101 may display the parts of the indication 413a at the same visual prominence.

FIG. 4H is a flow diagram illustrating a method 450 for displaying a representation of a physical tool as guidance for indicating a location of the physical tool relative to a location associated with video feed according to some examples of the disclosure. It is understood that method 450 is an example and that more, fewer, or different operations can be performed in the same or in a different order. Additionally, the operations in method 450 described below are, optionally, implemented by running one or more functional modules in an information processing apparatus such as general-purpose processors (e.g., as described with respect to FIG. 2) or application specific chips, and/or by other components of FIG. 2.

Therefore, according to the above, some examples of the disclosure are directed to a method (e.g., method 450 of FIG. 4H) including, at an electronic device in communication with one or more displays and one or more input devices, including a camera, presenting (452), via the one or more displays, a view of a physical environment of the electronic device from a viewpoint of the one or more displays in the physical environment, the view of the physical environment including an external view of a physical object, and a first physical tool, different from the camera. In some examples, the method 450 includes while presenting the view of the physical environment, displaying (454), via the one or more displays, a first user interface including video feed from the camera. In some examples, the method includes detecting (456) that a respective part of the first physical tool is at a location associated with the physical object that is absent from the video feed from the camera (e.g., detecting that the respective part is inside of the physical object but not in the field of view of the camera). In some examples, the method 450 includes in response to detecting that the respective part is at the location associated with the physical object that is absent from the video feed from the camera, displaying (458), via the one or more displays, a representation of the respective part of the first physical tool.

Additionally or alternatively, in some examples, the first physical tool includes a tip and a portion other than the tip, and the respective part is the tip.

Additionally or alternatively, in some examples, the first physical tool includes a tip and a portion other than the tip, and the respective part is the tip and the portion other than the tip.

Additionally or alternatively, in some examples, the first physical tool includes a tip and a portion other than the tip, and the respective part is the portion other than the tip.

Additionally or alternatively, in some examples, method 450 includes while displaying, via the one or more displays, the representation of the respective part of the first physical tool, detecting that the respective part of the first physical tool is at a location associated with the physical object that is in the video feed from the camera, and in response to detecting that the respective part of the first physical tool is at that location associated with the physical object that is in the video feed from the camera, ceasing displaying at least a portion of the representation of the respective part of the first physical tool.

Additionally or alternatively, in some examples, a length of the representation of the respective part of the first physical tool is based on a length of the respective part of the first physical tool.

Additionally or alternatively, in some examples, a length of the representation of the respective part of the first physical tool is based on a distance between a position within a field of view of the camera and the respective part of the first physical tool.

Additionally or alternatively, in some examples, method 450 includes detecting an input that changes the distance between the position within the field of view of the camera and the respective part of the first physical tool, and in response to detecting the input that changes the distance between the position within the field of view of the camera and the respective part of the first physical tool: in accordance with a determination that the input increases the distance between the position within the field of view of the camera and the respective part of the first physical tool, increasing the length of the representation of the respective part of the first physical tool, and in accordance with a determination that the input decreases the distance between the position within the field of view of the camera and the respective part of the first physical tool, decreasing the length of the representation of the respective part of the first physical tool.

Additionally or alternatively, in some examples, the representation of the respective part of the first physical tool is displayed outside of the first user interface that includes the video feed from the camera.

Additionally or alternatively, in some examples, displaying the representation of the respective part of the first physical tool outside the first user interface includes displaying the representation of the respective part of the first physical tool at a location that is based on a spatial arrangement between the first physical tool and the camera in the physical environment.

Additionally or alternatively, in some examples, method 450 includes while the respective part of the first physical tool is in the video feed from the camera, displaying, via the one or more displays and in the first user interface, an indication of a distance between the respective part of the first physical tool and a respective internal part of the physical object.

Additionally or alternatively, in some examples, in accordance with a determination that the distance is a first distance, the indication has a first appearance, and in accordance with a determination that the distance is a second distance, different from the first distance, the indication has a second appearance, different from the first appearance.

Additionally or alternatively, in some examples, the camera is a laparoscopic camera, the physical object is a body (e.g., of a human), and the first physical tool is a surgical instrument.

Additionally or alternatively, in some examples, the electronic device includes a head-mounted display system (e.g., the one or more displays are part of the head-mounted display system) and the one or more input devices include one or more sensors that are configured to detect an orientation and/or positioning of the first physical tool relative to the physical object.

Attention is now directed towards examples of an electronic device (e.g., computer system 101) displaying suggestions for changing a pose of a camera to a predetermined pose relative to a physical object in accordance with some examples of the disclosure.

In some cases, the electronic device 101 stores image data (e.g., captured images) detected by the camera 312 while the camera 312 is inside of physical object 310. In some cases, the electronic device 101 utilizes the stored image data to assist with moving the camera 312 back to a predetermined pose (e.g., a predetermined position and/or orientation). For example, at a first time, while the camera 312 has a predetermined pose (e.g., position and/or orientation) relative to the physical object 310 and/or while a first portion of the physical object 310 is in the field of view 313 of the camera 312 without a second portion of the physical object 310 being in the field of view 313 of the camera 312, the electronic device 101 detects a request to capture image data.

In response, while the camera 312 has the predetermined pose, the electronic device 101 captures the image data via the camera 312. After capturing the image data, the camera 312 may be moved to a different location that is inside or outside of the physical object 310. In some cases, it is desirable to return the camera 312 back to having the predetermined pose after the camera 312 has left the predetermined pose relative to the physical object 310. For example, at a second time, after the first time described above, the camera 312 is moved to outside of the physical object 310, and then at a third time, after the second time, it is desirable to move the camera 312 back to inside of the physical object 310 and specifically to having the predetermined pose relative to the physical object 310 so that the first portion of the physical object 310 described above is observed again in the camera feed. For example, it may be desirable to move the camera 312 back to having the predetermined pose so that the first portion of the physical object 310 may be in the field of view 313 of the camera 312 (e.g., the predetermined pose may be the optimal pose for viewing and/or operating on the first portion of the physical object 310). Some present examples provide for assisting with moving the camera back to a predetermined pose.

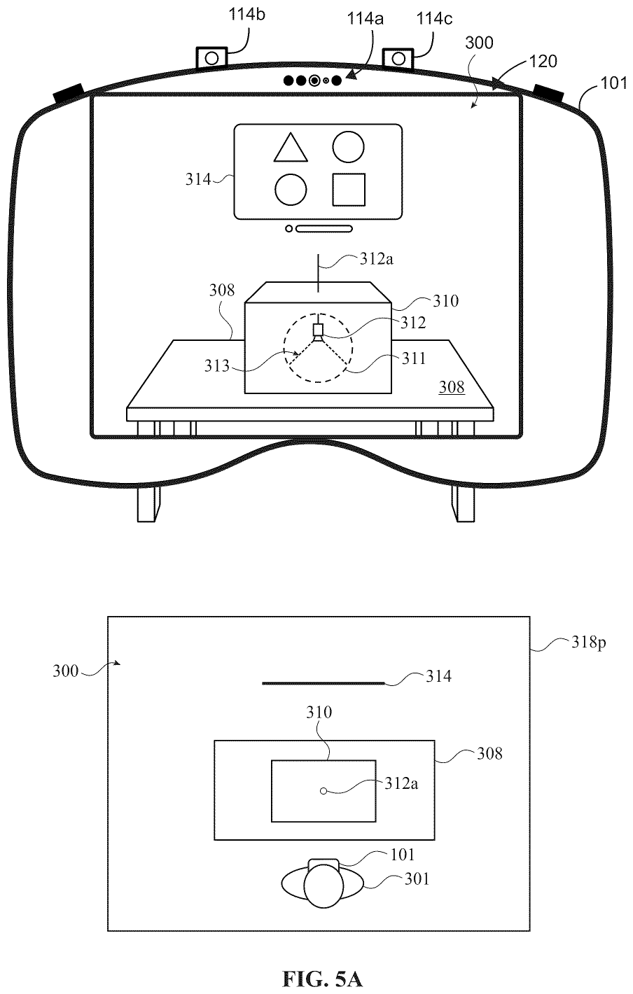

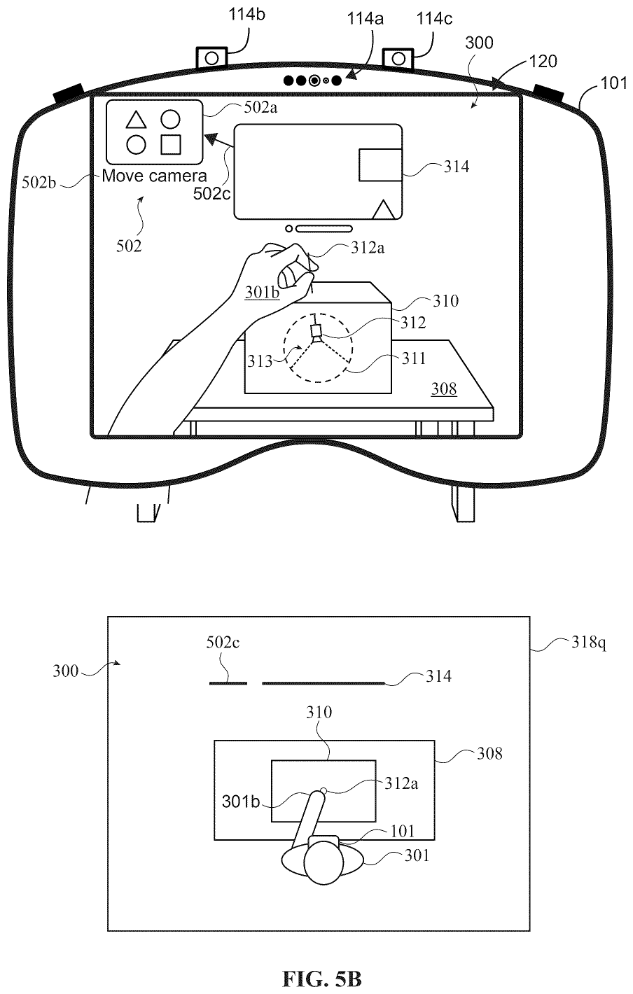

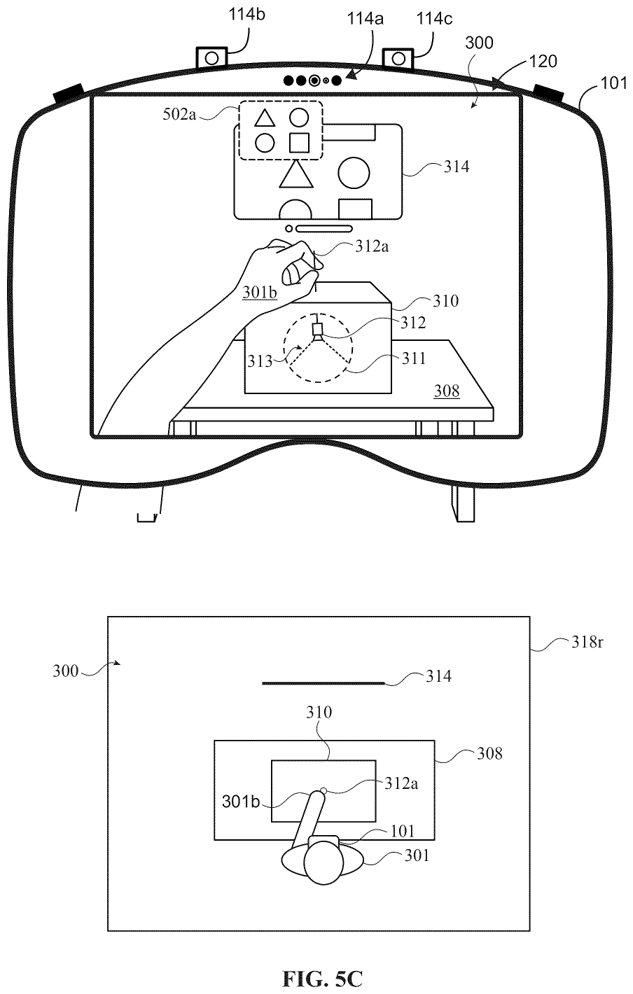

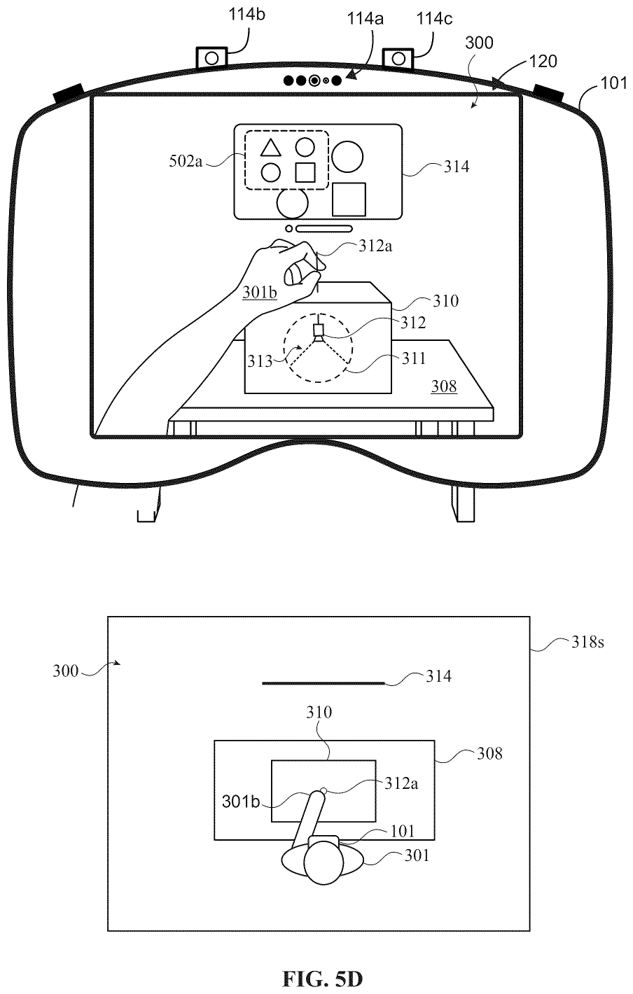

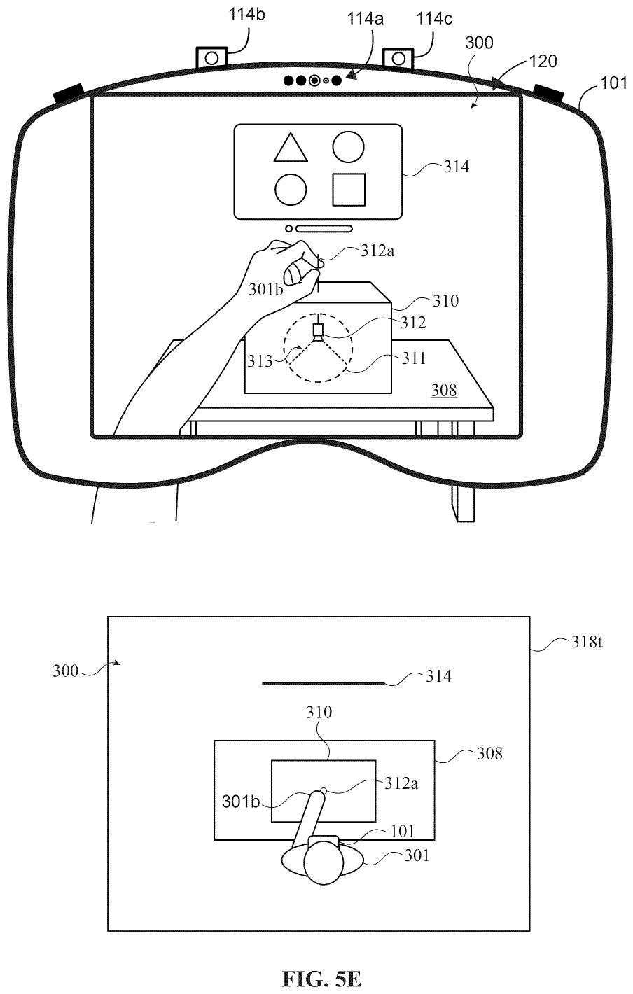

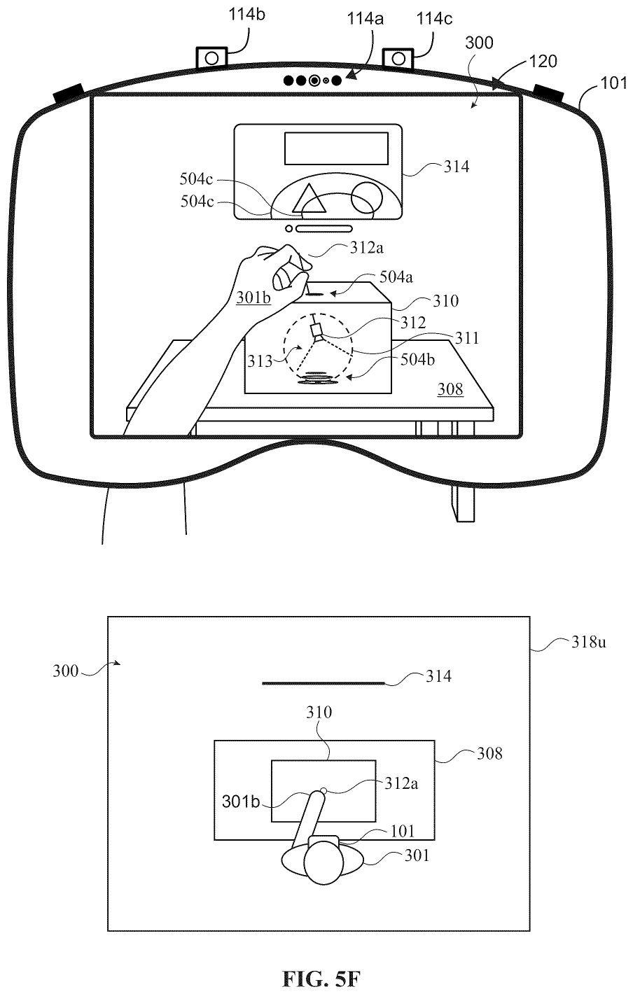

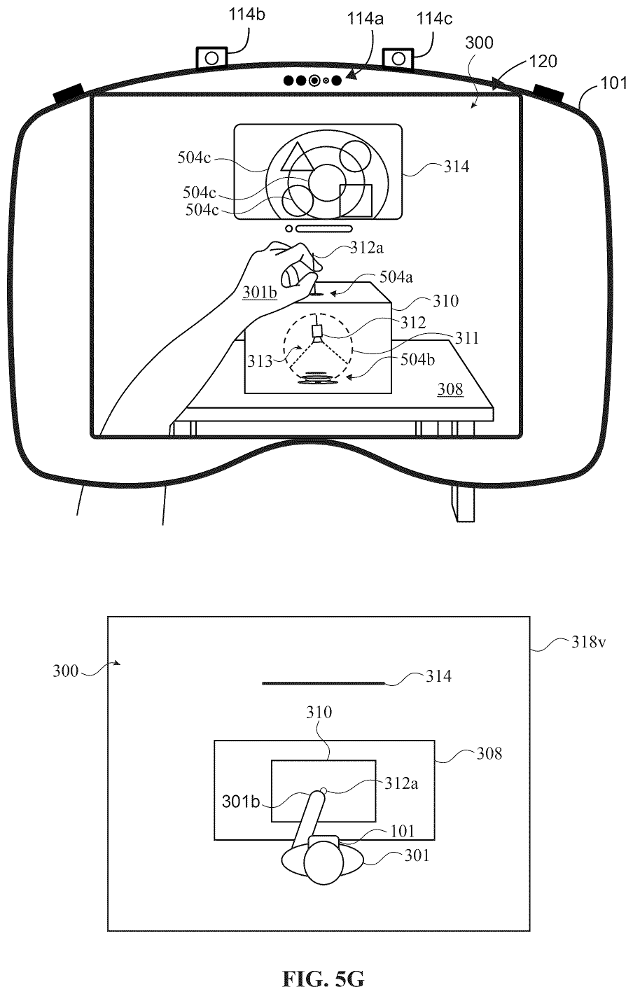

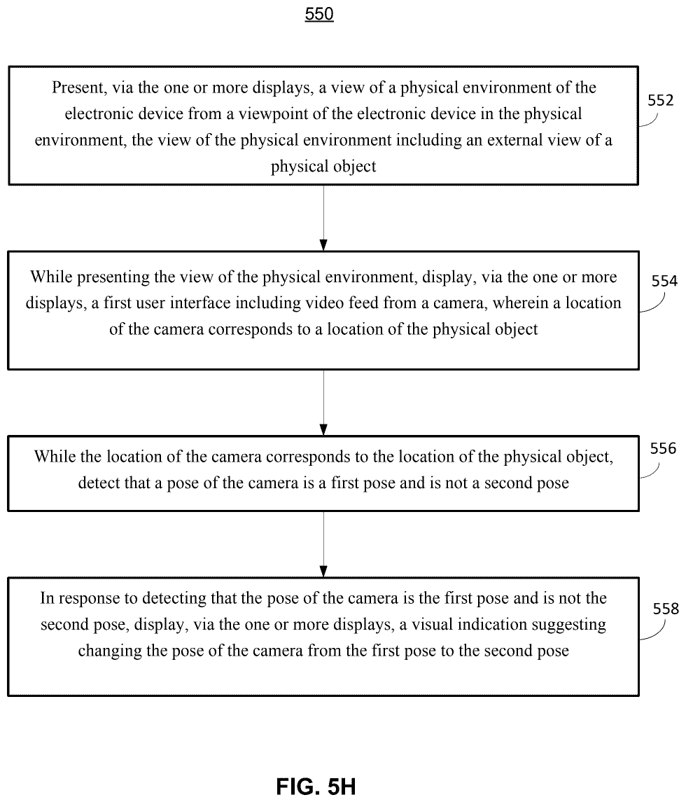

FIGS. 5A-5G illustrate examples of an electronic device displaying suggestions for changing a pose of a camera to a predetermined pose based on image data captured by the camera according to some examples of the disclosure.

For the purpose of illustration, FIGS. 5A-5G include respective top-down views 318p-318v of the three-dimensional environment 300 that indicate the positions of various objects (e.g., real and/or virtual objects) in the three-dimensional environment 300 in a horizontal dimension and a depth dimension. The top-down view of the three-dimensional environment 300 further includes an indication of the viewpoint of the user 301 of the electronic device 101. For example, in FIG. 5A, the electronic device 101 displays the view of the three-dimensional environment 300 visible through the display 120 from the viewpoint of the user 301 illustrated in the top-down view 318p of the three-dimensional environment 300.

In FIG. 5A, the camera 312 is inside physical object 310. In FIG. 5A, the camera 312 has a first pose (e.g., a first position and/or orientation) relative to the physical object 310. In FIG. 5A, the live camera feed user interface 314 shows image data captured by the camera 312 that is inside physical object 310 based on the camera 312 having the first pose. Were the camera 312 to have a first respective pose in the physical object 310, the live camera feed user interface 314 may show live images of the inside of physical object 310 from the perspective of the camera 312 having the first respective pose, and were the camera 312 to have a second respective pose in the physical object 310, different from the first respective pose in the physical object 310, the live camera feed user interface 314 may show live images of the inside of physical object 310 from the perspective of the camera 312 having the second respective pose. The first and second respective poses described above optionally correspond to different depths inside the physical object, different angular orientations, different lateral positions inside the physical object, and/or otherwise differences in locations of the camera 312 inside of the physical object 310 (e.g., differences in where the camera 312 is capturing images inside of the physical object 310).

In some examples, while the camera 312 has a respective pose, the electronic device 101 detects an input for capturing and saving one or more images captured by the camera 312. In response, the electronic device 101 may capture and save the one or more images in accordance with the input. For example, in FIG. 5A, the first pose may be the respective pose, and the electronic device 101 detects the input for capturing and saving one or more images captured by the camera 312 while the camera 312 has the illustrated pose. Continuing with this example, in response to the input, the electronic device 101 in FIG. 5A optionally captures and saves the one or more images captured by the camera 312.

In some cases, after capturing and saving one or more images captured by the camera 312 in the first pose in FIG. 5A, the camera 312 is moved such that it no longer has the first pose relative to the physical object 310. For example, a person optionally moves the camera 312 to outside of the physical object 310 or to another pose within the physical object 310. In some cases, after the camera 312 is moved away from the first pose, it is desirable to move the camera 312 back to the first pose inside of the physical object 310. For example, while the camera 312 has the first pose, a reference (e.g., a reference surface, object, or another reference in the physical object 310) may be shown at a first position (optionally with a first orientation) in the live camera feed user interface 314 and it may be desirable to move the camera 312 so that the reference might again be in the live camera feed user interface 314 at the first position. As such, example methods and systems that provide for guiding the camera back to having a previous pose may be useful.

In some examples, the electronic device 101 displays indications that guide placement of the camera 312 back to having the first pose relative to the physical object 310, such as shown in FIGS. 5B-5D.

In FIG. 5B, the pose of the camera 312 is different from the first pose of FIG. 5A. In some examples, a determination is made that the pose of camera 312 is different from the first pose of FIG. 5A based on what is shown in the live camera feed user interface 314. For example, the live camera feed user interface 314 in FIG. 5B shows different portions of physical object 310 in the field of view 313 of camera 312 than in FIG. 5A. In some examples, the determination is made based on image data of camera part 312a detected via external image sensors 114b/114c, as described above with reference to FIG. 4B. In FIG. 5B, the electronic device 101 displays a visual indication 502 that guides placement of the camera 312 back to the first pose illustrated in FIG. 5A. In FIG. 5B, the visual indication 502 includes a captured image 502a that was captured by the camera 312 when the camera 312 had the first pose (e.g., as in FIG. 5A), textual content 502b, and arrow 502c. For example, captured image 502a is a capture of the live camera feed user interface 314 in FIG. 5A. In some examples, in FIG. 5B, the captured image 502a is smaller in size than the live camera feed user interface 314. In some examples, a size of the captured image 502a changes (e.g., increases or decreases) as a function of distance between the captured image 502a and the live camera feed user interface 314. For example, as a difference in pose (e.g., a difference in position and/or orientation) between a current pose of the camera 312 and the first pose of the camera 312 is reduced, the electronic device 101 optionally increases or reduces a size of the captured image 502a in accordance with the reduced difference in pose. As another example, as a difference in pose between a current pose of the camera 312 and the first pose of the camera 312 is increased, the electronic device 101 optionally increases or reduces a size of the captured image 502a in accordance with the increased difference in pose. In some examples, a size of the captured image 502a is constant with respect to differences in pose between the current pose of the camera 312 and the first pose of the camera 312.

In FIG. 5B, the electronic device 101 displays the captured image 502a at a location relative to the live camera feed user interface 314 that is based on a distance offset between the current pose of the camera 312 and the first pose of the camera 312 in FIG. 5A. For example, in FIG. 5B, a distance between display of the captured image 502a and the live camera feed user interface 314 is optionally based on an amount of offset between the current pose of the camera and the first pose of the camera 312 in FIG. 5A. For example, if the current pose of the camera is offset (e.g., laterally offset) from the first pose by a first amount (e.g., 2 cm or another amount), the electronic device 101 would display the captured image 502a and the live camera feed user interface 314 having a first separation distance, and if the current pose of the camera 312 is offset (e.g., laterally offset) from the first pose by a second amount (e.g., 4 cm or another amount), different from the first amount, the electronic device 101 would display the captured image 502a and the live camera feed user interface 314 having a second separation distance that is different from the first separation distance. In some examples, the greater the offset between the current pose and the first pose, the greater separation distance between display of captured image 502a and live camera feed user interface 314. As such, in some examples, the separation distance indicates an amount of movement needed to move the camera 312 for the camera 312 to have the first pose.

Additionally, in FIG. 5B, the electronic device 101 displays the captured image 502a at a location relative to the live camera feed user interface 314 that is based on a direction associated with the offset between the current pose of the camera and the first pose of the camera 312 in FIG. 5A. For example, in FIG. 5B, the electronic device 101 optionally displays captured image 502a at a location that is northwest of the live camera feed user interface 314 to suggest moving the camera 312 in a corresponding direction in the physical object 310 for the camera 312 to have the first pose. For example, if the current pose of the camera is offset from the first pose in a first corresponding direction (e.g., relative to the first pose), the electronic device 101 would display the captured image 502a offset from the live camera feed user interface 314 in a first direction relative to the live camera feed user interface 314 (e.g., relative to a center of the live camera feed user interface 314), and if the current pose of the camera is directionally offset from the first pose in a second corresponding direction, different from the first corresponding direction, the electronic device 101 would display the captured image 502a offset from the live camera feed user interface 314 in a second direction (e.g., relative to a center of the live camera feed user interface 314) that is different from the first direction. As such, in some examples, where the electronic device 101 displays the captured image 502a relative to the live camera feed user interface 314 is based on a direction associated with the offset between the current pose of the camera 312 and the first pose of the camera 312, which further optionally indicates a suggested direction by which to move the camera 312 so that it might have the first pose.

Additionally, in FIG. 5B, the electronic device 101 displays textual content 502b and arrow 502c suggesting movement of the camera 312. In FIG. 5B, the textual content 502b indicates “move camera” and arrow 502c points in a direction that corresponds to the direction by which the camera 312 should be moved within the physical object 310 so that the camera 312 can have the first pose. As described above, in some examples, the electronic device 101 displays captured image 502a at a location relative to live camera feed user interface 314 that is based on a direction associated with an offset between the current pose of the camera 312 and the first pose of the camera 312. Similarly, in some examples, the electronic device 101 displays arrow 502c at a location relative to the live camera feed user interface 314 that is based on the direction associated with the offset between the current pose of the camera 312 and the first pose of the camera 312. For example, were the direction associated with the offset to be a first direction, the electronic device 101 may display the arrow 502c at a first location relative to the live camera feed user interface 314 based on that first direction, and were the direction associated with the offset to be a second direction, different from the first direction, the electronic device 101 may display the arrow 502c at a second location, different from the first location, relative to the live camera feed user interface 314 based on that second direction. Similarly, in some examples, the direction that the arrow 502c points is based on the direction associated with the offset between the current pose of the camera 312 and the first pose of the camera 312. For example, were the direction associated with the offset to be a first direction, the electronic device 101 may display the arrow 502c at a first location relative to the live camera feed user interface 314 and pointing in a first respective direction based on that first direction, and were the direction associated with the offset to be a second direction, different from the first direction, the electronic device 101 may display the arrow 502c at a second location, different from the first location, relative to the live camera feed user interface 314 and pointing in a second respective direction, different from the first respective direction, based on that second direction. In some examples, the arrow 502c may lie along a vector extending from a center of live camera feed user interface 314 to a center of the captured image 502a. In the illustrated example of FIG. 5B, the electronic device 101 displays the arrows 502c pointing toward the location of display of the captured image 502a.

From FIG. 5B to FIG. 5C, the electronic device 101 has detected a change in a pose of the camera 312. A difference in pose between the current pose of the camera 312 in FIG. 5C and the first pose of the camera 312 in FIG. 5A is less than the difference in pose between the pose of the camera 312 in FIG. 5B and the first pose of the camera 312 in FIG. 5A (e.g., in FIG. 5C, though the camera does not have the first pose, the camera 312 is more aligned with the first pose than the alignment between the current pose of the camera 312 and the first pose in FIG. 5B). In response, the electronic device 101 has moved the location of display of the captured image 502a toward the live camera feed user interface 314, as shown from FIG. 5B to FIG. 5C. In addition, in the illustrated example of FIG. 5C, the electronic device 101 ceased display of the textual content 502b and arrow 502c described above with reference to FIG. 5B. In some examples, the electronic device 101 alternatively maintains display of the textual content 502b and/or arow 502c described above with reference to FIG. 5B even while displaying the illustrated example of FIG. 5C. From FIG. 5B to FIG. 5C, the electronic device 101 has reduced a distance between the captured image 502a and the live camera feed user interface 314 (e.g., a center of live camera feed user interface 314) in accordance with the reduced offset (e.g., a reduced distance) between the current pose of the camera in FIG. 5C and the first pose of the camera in FIG. 5A compared with the offset (e.g., distance) between the current pose of the camera in FIG. 5B and the first pose of FIG. 5A. Further, in the illustrated example of FIG. 5C, a portion of the captured image 502a overlaps a portion of the live camera feed user interface 314. In some examples, the portion of the captured image 502a that overlaps the portion of the live camera feed user interface 314 is partially transparent so that the portion of the live camera feed user interface 314 is at least partially visible through the portion of the captured image 502a.

From FIG. 5C to FIG. 5D, the electronic device 101 has detected further change in pose of the camera 312 (e.g., the camera 312 has moved due to input from hand 301b). From FIG. 5C to FIG. 5D, a difference in pose between the current pose of the camera 312 in FIG. 5D and the first pose of the camera 312 in FIG. 5A is less than the difference in pose between the pose of the camera 312 in FIG. 5C and the first pose of the camera 312 in FIG. 5A (e.g., in FIG. 5D, though the camera 312 does not have the first pose, the camera 312 is more aligned with the first pose than the alignment between the current pose of the camera 312 and the first pose in FIG. 5C). In response, the electronic device 101 has moved the location of display of the captured image 502a toward the live camera feed user interface 314, as shown from FIG. 5C to FIG. 5D. For example, a difference between the current pose of the camera 312 and the first pose of the camera 312 in FIG. 5D may be less than a difference between the current pose of the camera 312 and the first pose of the camera 312 in FIG. 5C. As such, from FIG. 5C to FIG. 5D, the electronic device 101 further reduces a distance between the captured image 502a and the live camera feed user interface 314 (e.g., a center of live camera feed user interface 314) in accordance with the reduced offset (e.g., reduced distance) between the current pose of the camera in FIG. 5D and the first pose of the camera in FIG. 5A (e.g., compared with the offset (e.g., distance) between the current pose of the camera in FIG. 5C and the first pose of FIG. 5A). For example, an overlap between the captured image 502a and the live camera feed user interface 314 increases relative to the viewpoint of the electronic device 101, as shown in FIG. 5D.

As mentioned above, in the illustrated example of FIG. 5D, captured image 502a overlaps a portion of the live camera feed user interface 314. In some examples, in FIG. 5D, the captured image 502a is partially transparent so that the portion of the live camera feed user interface 314 that it overlaps is at least partially visible. Note that the electronic device 101 optionally changes a visual prominence (e.g., an amount of transparency or brightness) of captured image 502a based on an amount of offset (e.g., directional or distance offset) between the current pose of the camera and the first pose of the camera 312 in FIG. 5A. For example, a visual prominence of captured image 502a in FIG. 5B is optionally different from (e.g., less than) a visual prominence of captured image 502a in FIG. 5C. As another example, a visual prominence (e.g., an amount of transparency or brightness) of captured image 502a in FIG. 5C is optionally different from (e.g., less than) a visual prominence (e.g., an amount of transparency or brightness) of captured image 502a in FIG. 5D. In some examples, the electronic device 101 reduces a visual prominence of the captured image 502a as the captured image 502a is moved toward the live camera feed user interface 314. Thus, in some examples, the visual prominence of the captured image 502a is indicative of an amount of offset between the current pose of the camera 312 and the first pose of the camera 312 in FIG. 5A.

In some examples, when the current pose of the camera is within a threshold of the first pose of the camera 312, the electronic device 101 ceases display of the captured image 502a. For example, were the electronic device 101 to detect further movement of the camera that further aligns the current pose of the camera 312 (e.g., from that in FIG. 5D) to the first pose of the camera in FIG. 5A, the current pose of the camera 312 would be within the threshold of the first pose of the camera 312 and the electronic device 101 may cease displaying the captured image 502a and maintain display of the live camera feed user interface 314 which would now be a stream of the camera feed while the camera 312 is within the threshold of the first pose. For example, were the electronic device 101 to detect movement of the camera 312 to within the threshold of the first pose of the camera 312, the electronic device 101 may display live camera feed user interface 314 including the feed of the camera at the current pose of the camera that is within the threshold of the first pose, without displaying the captured image 502a. From FIG. 5D to 5E, the camera 312 is moved (e.g., via input from hand 301b illustrated in top-down view 518d) from its pose in FIG. 5D to the first pose. In response, in FIG. 5E, the electronic device 101 ceases display of the visual indication suggesting changing the pose of the camera 312, since the camera 312 is in the first pose in FIG. 5E just like in FIG. 5A.

Additionally or alternatively, in some examples, the electronic device 101 may display different visual indications suggesting moving the camera in addition to or instead of the visual indications 502a-502c described above. In some examples, the electronic device 101 displays visual indications 504a and 504b, which are overlaid on an external surface of the physical object 310, and visual indication 504c, which is in the live camera feed user interface 314, such as shown in FIG. 5F. In FIG. 5F, visual indication 504a includes a highlight on the entry point of the camera 312 into the physical object 310 and visual indication 504b is illustrated as rings having different vertical depths. The visual indications 504a/504b may be displayed to guide placement of the camera 312 to have the first pose. For example, the visual indication 504b may be displayed to suggest movement of the camera part 312a toward facing the center of the rings of visual indication 504b. In some examples, the electronic device 101 maintains the visual appearance of visual indication 504b when movement of camera 312 is detected. In some examples, the electronic device 101 modifies display of the visual indication 504a and/or visual indication 504b in accordance with movement of the camera 312. Further, in FIG. 5F, the electronic device 101 displays visual indication 504c in the live camera feed user interface 314. In FIG. 5F, the electronic device 101 displays visual indication 504c to guide placement of the camera 312 back to the first pose of FIG. 5A. In FIG. 5F, the visual indication 504c includes rings that may be at different depths (or at the same depth) in the field of view 313 of the camera 312 and that may be displayed in the live camera feed user interface 314 for guiding placement of the camera 312. The rings are optionally for guiding placement of the camera 312 toward facing a center of the rings of the visual indication 504c. For example, the rings are visually suggestive of moving the camera 312 so that the center of the rings is displayed at the center of the live camera feed user interface 314 from the viewpoint of the electronic device 101. For example, in FIG. 5F, the location of display of the visual indication 504c may suggest moving the camera 312 down and/or laterally in a direction that would move the center of the rings to the center of the live camera feed user interface 314, thus moving the camera 312 toward having the first pose. For example, from FIG. 5F to FIG. 5G, the electronic device 101 detects movement of the camera 312 toward the first pose, and updates display of the visual indication 504c in the live camera feed user interface 314 correspondingly, which now includes the center of the rings of visual indication 504c being in the live camera feed user interface 314. In some examples, the electronic device 101 animates movement of the rings of visual indication 504c in live camera feed user interface 314 in accordance with movement of the camera 312. For example, in response to the detected movement of the camera 312 from FIG. 5F to FIG. 5G, the electronic device 101 may which portions of the rings of visual indication 504c at different locations to correspond to the new field of view 313 of the camera 312 that is due to the movement of the camera 312. As such, in some examples, the electronic device 101 displays visual indications (e.g., rings) in the live camera feed user interface 314 and visual indications on the external view of the physical object 310 that is presented via display 120 for suggesting movement of the camera to the first pose.

In some examples, the electronic device 101 maintains a spatial arrangement of the visual indication 504c relative to the physical object 310. For example, from FIG. 5F to FIG. 5G, the camera has been moved to being more aligned with the first pose of FIG. 5A, and though the visual indication 504c is displayed differently in the live camera feed user interface 314 in FIG. 5G than in FIG. 5F (e.g., the visual indication includes two rings in FIG. 5F and includes three rings in FIG. 5G), the visual indication 504c has maintained its spatial arrangement relative to the physical object 310. In some examples, the electronic device 101 displays respective rings having different visual prominences (e.g., different contrasts, brightness, saturations, opacities, etc.) based on a depth in the physical object 310 to which the respective ring corresponds and/or based on a distance between the camera and the respective ring. For example, if a distance between the camera 312 and a second ring is less than a distance between the camera 312 and a first ring, different from the second ring, the electronic device 101 may display the first ring as having a greater visual prominence than the second ring. In some examples, the ring that is closest in depth to the camera 312 is displayed with the greatest visual prominence of the plurality of rings in the respective visual indication. In some examples, the ring that is furthest away in depth from the camera 312 is displayed with the least visual prominence of the plurality of rings in the respective visual indication.

FIG. 5H is a flow diagram illustrating a method 550 for displaying a visual indication suggesting changing a pose of a camera from a first pose to a second pose according to some examples of the disclosure. It is understood that method 550 is an example and that more, fewer, or different operations can be performed in the same or in a different order. Additionally, the operations in method 550 described below are, optionally, implemented by running one or more functional modules in an information processing apparatus such as general-purpose processors (e.g., as described with respect to FIG. 2) or application specific chips, and/or by other components of FIG. 2.

Therefore, according to the above, some examples of the disclosure are directed to a method (e.g., method 550 of FIG. 5H) including, at an electronic device in communication with one or more displays and one or more input devices, including a camera, presenting (552), via the one or more displays, a view of a physical environment of the electronic device from a viewpoint of the electronic device in the physical environment, the view of the physical environment including an external view of a physical object, while presenting the view of the physical environment, displaying (554), via the one or more displays, a first user interface including video feed from the camera, wherein a location of the camera corresponds to a location of the physical object, while the location of the camera corresponds to the location of the physical object, detecting (556) that a pose of the camera is a first pose and is not a second pose, and in response to detecting that the pose of the camera is the first pose and is not the second pose, displaying (558), via the one or more displays, a visual indication suggesting changing the pose of the camera from the first pose to the second pose.

Additionally or alternatively, in some examples, the visual indication includes a suggested direction of movement of the camera to place the camera in the second pose.

Additionally or alternatively, in some examples, the camera was previously posed in the second pose, and detecting that the pose of the camera is the first pose and is not the second pose includes a detection that first image data detected by the camera while the camera had the second pose is different from second image data detected by the camera while the camera has the first pose.

Additionally or alternatively, in some examples, the visual indication includes an image captured via the camera while the camera previously had the second pose.

Additionally or alternatively, in some examples, the image is displayed outside of the first user interface.

Additionally or alternatively, in some examples, displaying the image outside of the first user interface includes displaying the image based on a difference between one or more spatial properties of the first pose and one or more spatial properties of the second pose.

Additionally or alternatively, in some examples, the method includes while detecting the pose of the camera is changing from the first pose to the second pose, moving the image relative to the first user interface.

Additionally or alternatively, in some examples, a location of the display of the image and a location of the display of the first user interface overlap.

Additionally or alternatively, in some examples, the method includes reducing in visual prominence the image when the camera is changed to the second pose.

Additionally or alternatively, in some examples, the visual indication includes a textual suggestion to move the camera, and a direction element indicating a direction to move the camera to pose the camera in the second pose from the first pose.

Additionally or alternatively, in some examples, the visual indication includes a representation of one or more concentric rings that are displayed in the first user interface.

Additionally or alternatively, in some examples, the method includes concurrently displaying, via the one or more displays, a second visual indication on the external view of the physical object with the visual indication, wherein the second visual indication includes one or more indications at one or more depths on the physical object suggestive of a path that the camera needs to be moved along to be in the first pose.

Additionally or alternatively, in some examples, the first pose is not aligned with the second pose by a first amount, and the method includes detecting a first movement of the camera that results in the camera having a third pose that is more aligned with the first pose than the first amount, and in response to detecting the first movement of the camera, moving the representation of the one or more concentric rings relative to the first user interface, including maintaining a spatial arrangement between the representation of the one or more concentric rings relative to the physical object.

Additionally or alternatively, in some examples, the camera is a laparoscopic camera and the physical object is a body of a human.

Additionally or alternatively, in some examples, the one or more displays includes a head-mounted display system.

Attention is now directed towards examples of an electronic device displaying image data and a live camera feed from a camera and scrubbing through the image data in accordance with changes to a pose of the camera relative to a physical object.

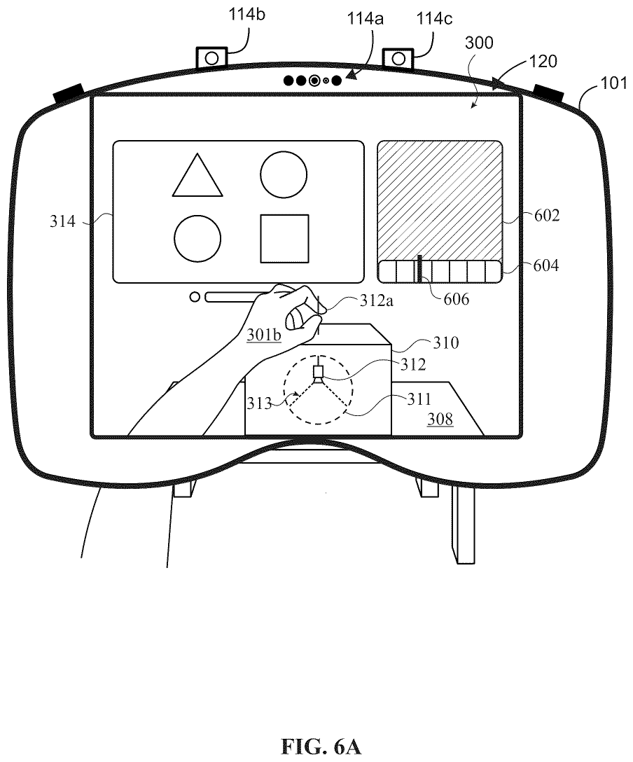

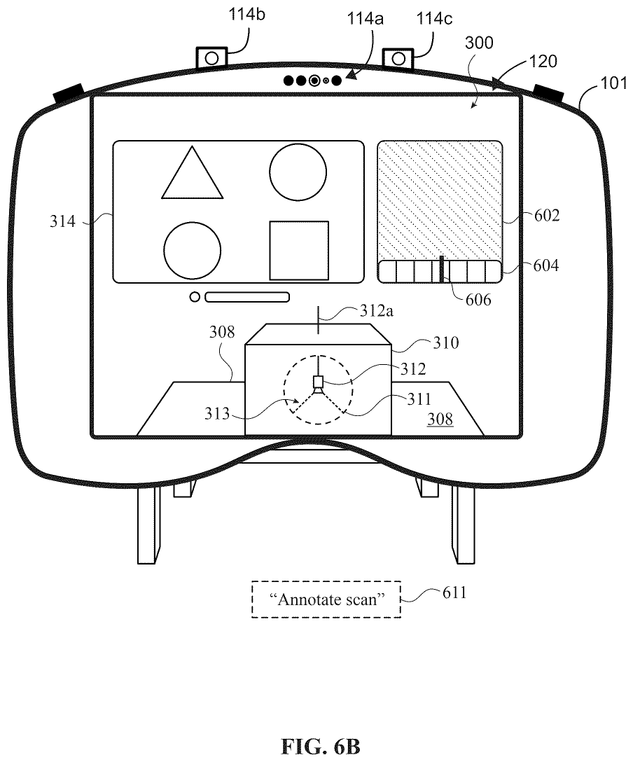

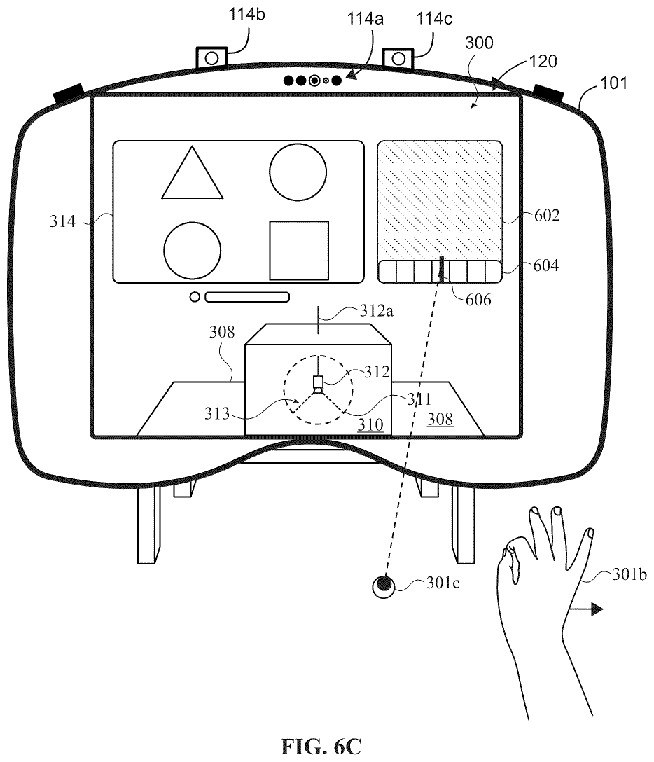

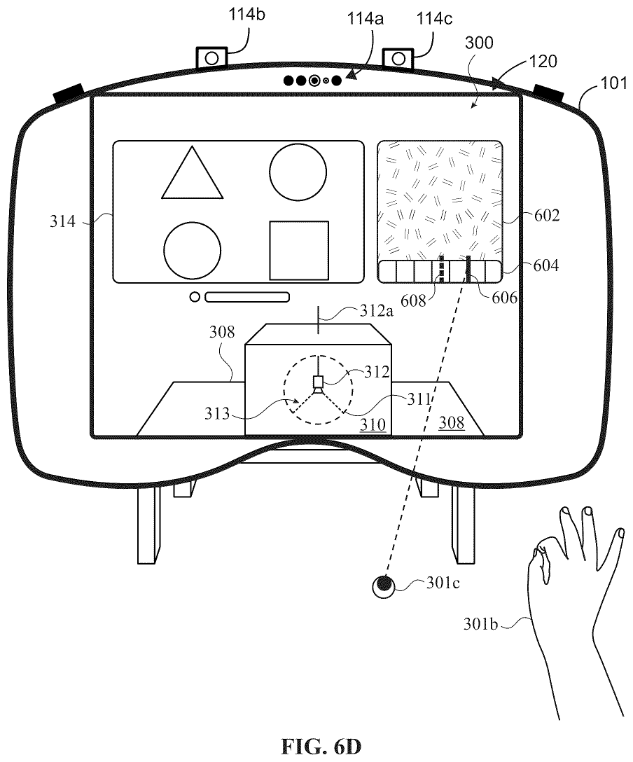

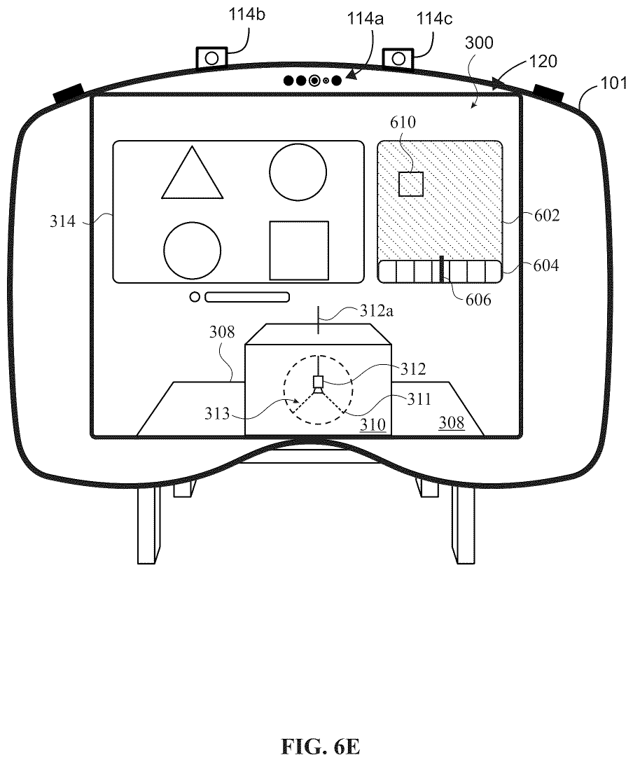

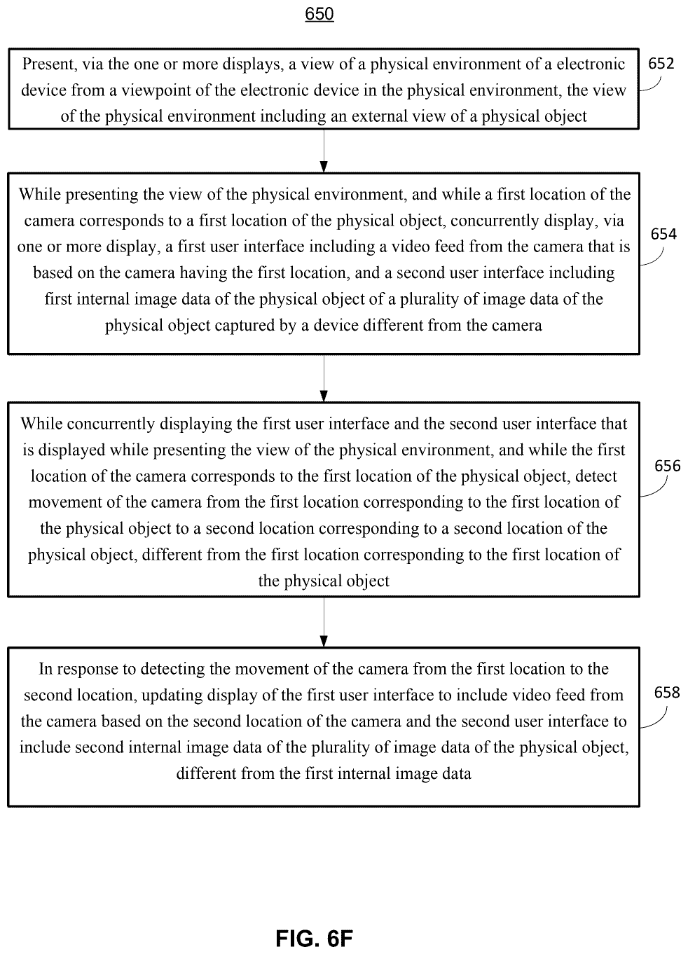

As mentioned above, some examples of the disclosure are directed to an electronic device displaying a live camera feed and image data, and scrubbing through the image data in accordance with changes to a pose of the camera relative to a physical object. For instance, in some examples, an electronic device automatically scrubs through scans (e.g., image data) based on change in a depth position of a camera relative to the physical object. FIGS. 6A-6E illustrate examples of an electronic device scrubbing through image data while displaying a live camera feed user interface according to some examples of the disclosure.

Note that in FIGS. 6A-6E, the electronic device 101 displays the view of the three-dimensional environment 300 visible through the display 120 from the viewpoint of the user 301 in the respective figure, and that the relative placements of live camera feed user interface 314 and the image data user interface 602 may be similar to (e.g., same as) the placements of the live camera feed user interface 314 and the box 322a in FIG. 3C (e.g., as shown in the top-down view 318c of FIG. 3C), respectively. For example, the live camera feed user interface 314 and the image data user interface 602 may be at a greater depth from the user 301 than the physical object 310 and the table 308 in FIGS. 6A-6E.

In FIG. 6A, the electronic device 101 displays live camera feed user interface 314 and an image data user interface 602. The live camera feed user interface 314 shows camera feed from the camera 312 that is inside of the physical object 310. The image data user interface 602 shows image data captured by a device different from the camera 312. For example, the image data shown in image data user interface 602 is optionally an MRI scan captured by a magnetic resonance imaging (MRI) device. In some examples, the image data was captured before camera 312 is inside of (e.g., inserted into) the physical object 310. For example, the image data was captured while the physical object 310 was undergoing MRI scans. In some examples, the image data is captured and then is stored as associated specifically with the physical object 310 (e.g., and/or the patient to which the physical object 310 belongs), such that a user would need authorization to view the image data that is associated with the physical object 310. In FIG. 6A, image data user interface 602 includes a scrubber bar 604 for scrubbing through a plurality of scans captured by the device. Scrubber bar 604 includes a current position indicator 606 which indicates the position in the plurality of scans that the displayed scan in the image data user interface 602 corresponds. In FIG. 6A, the current position indicator 606 is at a first position in the scrubber bar 604. In some cases, the plurality of scans of the physical object 310 includes different scans of the physical object captured at different depths or otherwise different arrangements between the physical object 310 and the device that captured the scans of the physical object 310. In some cases, it is desirable to show different views of the physical object 310, such as different scans of the physical object 310 to assist in performance of one or more operations on the physical object 310.

In some examples, the electronic device 101 displays the image data user interface 602 concurrently with the live camera feed user interface 314 in response to input directed to the user interface element 324a in FIG. 3C. For example, while the electronic device 101 is presenting the environment illustrated in FIG. 3C, in which user interface element 324b is selected, and in which live camera feed user interface 314, box 322a, and 3D object 322b are being displayed, the electronic device 101 may detect user input selecting user interface element 324a. In response, the electronic device 101 ceases display of box 322a and 3D object 322b, and displays the image data user interface 602, as shown in FIG. 6A.

In some cases, it is desirable for the electronic device 101 to automatically scrub through the plurality of scans in accordance with movement of the camera 312. For example, in FIG. 6A, while displaying image data user interface 602 showing a first scan that corresponds to a first pose of camera 312 in FIG. 6A (e.g., the camera being at a first depth in the physical object 310), the electronic device 101 may detect movement of the camera 312 to a second pose (e.g., to a second depth greater than the first depth), different from the first pose. For example, in FIG. 6A, hand 301b may move the camera 312 vertically downward in the physical object 310, thus changing a depth of the camera 312 relative to the physical object 310. In response, the electronic device 101 may scrub through the plurality of scans of the physical object in accordance with the detected change in pose, as shown from FIG. 6A to FIG. 6B.

From FIG. 6A to FIG. 6B, the current position indicator 606 in the scrubber bar 604 has moved from the first position illustrated in FIG. 6A to a second position (e.g., different from the first position) illustrated in FIG. 6B. In some examples, during the movement of the current position indicator 606, the electronic device 101 scrubs through the plurality of scans such that different scans (e.g., intermediate scans between the first position and the second position) are shown in the image data user interface 602 until the displayed scan in the image data user interface 602 corresponds to the scan at the second position of the current position indicator 606 in the scrubber bar 604. For example, while the current position indicator 606 is at its illustrated position in FIG. 6A, the image data user interface 602 may show a first scan of the plurality of scans, and while the current position indicator 606 is at its illustrated position in FIG. 6B, the image data user interface 602 may show a second scan of the plurality of scans that is different from the first scan.

The electronic device 101 may scrub through the plurality of scans in a direction based on a direction of movement of the camera 312. For example, in accordance with a determination that the movement of the camera is movement in a first direction (e.g., downward relative to the physical object 310), the electronic device 101 may scrub through the plurality of scans in a first respective direction. Continuing with this example, in accordance with a determination that the movement of the camera 312 is movement in a second direction (e.g., upward relative to the physical object 310), different from the first direction, the electronic device 101 may scrub through the plurality of scans in a second respective direction that is different from the first respective direction. For example, from FIG. 6A to 6B, the electronic device 101 may have scrubbed through the plurality of scans in the direction associated with the movement of the camera 312 downward relative to the physical object 310 and the current position indicator 606 in the scrubber bar 604 may have moved rightward due to the direction of movement of the camera 312. If the camera 312 were instead moved opposite the direction described above, the electronic device 101 would have scrubbed through the plurality of scans in the opposite direction (e.g., the current position indicator 606 in FIG. 6B would have been moved leftward of the location of the current position indicator 606 in FIG. 6A instead of rightward of the location of the current position indicator 606 in FIG. 6A). In some examples, rightward movement of the current position indicator 606 corresponds to scrubbing through scans that increase (e.g., consecutively increase) in zoom level and leftward movement of the current position indicator 606 corresponds to scrubbing through scans that decrease (e.g., consecutively decrease) in zoom level. For example, from FIG. 6A to 6B, the current position indicator 606 is moved rightward due to the increase in depth of the camera 312 relative to the physical object 310 and the resulting scan shown in image data user interface 602 in FIG. 6B is a scan that is of a greater zoom level than the zoom level of the scan in FIG. 6B.

In some cases, it is desirable to scrub through the plurality of scans independent of whether the camera 312 has moved. In some examples, the electronic device 101 provides for scrubbing through the plurality of scans independent of whether the camera 312 has moved, such as shown from FIG. 6C to FIG. 6D. For example, while displaying the image data user interface 602 concurrently with the live camera feed user interface 314, the electronic device 101 may detect an input (e.g., gaze 301c of the user 301 and/or hand 301b of the user 301 performing an air pinch gesture) directed at the scrubber bar 604 of the image data user interface 602 (e.g., a scan user interface), such as shown in FIG. 6C. For example, the input optionally requests movement of the current position indicator 606 in the scrubber bar 604 from the current position in the scrubber bar 604 to a different position in the scrubber bar 604. In some examples, the requested movement of the current position indicator 606 is in the same direction as the movement associated with the input. For example, were the input to include movement of the hand 301b of the user leftward, the requested movement of the current position indicator may be leftward, and were the input to include movement of the hand 301b of the user rightward, the requested movement of the current position indicator 606 may be rightward. In response to the input, the electronic device 101 may move the current position indicator 606 in the scrubber bar 604 to the different position and scrub through the plurality of the scans until the scan that corresponds to the different position of the current position indicator is reached, independent of a change in pose of the camera 312, as shown in FIG. 6D. In particular, in FIG. 6D, the pose of the camera 312 is the same as in FIG. 6C (e.g., the live camera feed user interface 314 is showing the same content), but the image data user interface 602 has changed in content to a different scan. For example, while the current position indicator 606 is at its illustrated position in FIG. 6C, the image data user interface 602 may show a first respective scan of the plurality of scans, and while the current position indicator 606 is at its illustrated position in FIG. 6D, the image data user interface 602 may show a second respective scan of the plurality of scans that is different from the first respective scan.

In some examples, when the input directed to the scrubber bar 604 is detected, the current position indicator 606 in the scrubber bar 604 is synchronized to the pose of the camera 312 (e.g., its current position corresponds to the current pose of the camera 312), as described with reference to FIGS. 6A and 6B. In some examples, in response to detecting the input directed to the scrubber bar 604, the current position indicator 606 in the scrubber bar 604 unlocks (e.g., ceases to be synchronized to the pose of the camera 312) and moves in accordance with the input directed to the scrubber bar 604 that requests its movement, as shown from FIG. 6C to FIG. 6D. In some examples, while the current position indicator 606 is not at the position in the scrubber bar 604 that corresponds to the current pose of the camera 312, the electronic device 101 displays a marker that indicates the position in the scrubber bar 604 that corresponds to the current pose of the camera 312, such as marker 608 in FIG. 6D. In some examples, while the current position indicator 606 is not at the position in the scrubber bar 604 that corresponds to the current pose of the camera 312, were the camera 312 to move to a pose that corresponds to the position of the marker 608 in the scrubber bar 604, the electronic device 101 may synchronize (e.g., lock) the current position indicator 606 to the current pose of the camera 312 such that were further movement of the camera 312 detected after the camera 312 has been moved to the pose that corresponds to the position of the marker 608 in the scrubber bar 604, the current position indicator 606 in the scrubber bar 604 would automatically move to maintain the correspondence.

In some examples, the scrubber bar 604 maintains display of marker 608 in the scrubber bar 604 even when the current position indicator 606 in the scrubber bar 604 is moved in response to user input directed at the scrubber bar 604. In some examples, if, while the current position indicator 606 is moving in accordance with the input directed to the scrubber bar 604, the current position indicator 606 is moved to the position of the marker 608 in the scrubber bar 604, the current position indicator 606 may lock to the location of the marker 608 (e.g., the current position indicator 606 may cease movement or become synchronized to the current pose of the camera 312, and the marker 608 may cease in display) and the image data user interface 602 would show the scan that corresponds to the current pose of the camera 312, which is the scan at the position of the current position indicator 606. In some examples, were the user 301 to request further movement of the current position indicator 606 after the current position indicator 606 is locked again to correspond to the current pose of the camera 312, the user may have to provide a second input to the electronic device 101 for scrubbing through the plurality of scans. In some examples, if, while the current position indicator 606 is moving in accordance with the input directed to the scrubber bar 604, the current position indicator 606 is moved to the position of the marker 608 in the scrubber bar 604, the current position indicator 606 may continue moving (e.g., in accordance with the input directed to the scrubber bar 604) without locking to the position of the marker 608 in the scrubber bar 604.

In some examples, when the input directed to the scrubber bar 604 is complete (e.g., when the gaze of the user 301 is no longer directed to the current position indicator 606 in the scrubber bar 604 and/or when the hand 301b is no longer in the pose (e.g., the air pinch pose)), the electronic device 101 maintains display of the scan corresponding to where the current position indicator 606 has been moved and does not scrub back through the plurality of scans to display the scan that corresponds to the current pose of the camera 312. In some examples, when the input directed to the scrubber bar 604 is complete (e.g., when the gaze of the user 301 is no longer directed to the current position indicator 606 in the scrubber bar 604 and/or when the hand 301b is no longer in the pose (e.g., the air pinch pose)), the electronic device 101 moves (e.g., automatically moves) the current position indicator back to the location in the scrubber bar 604 that corresponds to the current pose of the camera 312 and scrubs back to display the scan that corresponds to the current pose of the camera 312. For example, the input directed to the scrubber bar 604 may be complete in FIG. 6D while the current position indicator 606 is at its illustrated location, and in response to such completion, the electronic device 101 may automatically scrub through the plurality of scans to display the scan that corresponds to the current pose of the camera 312, which is the scan illustrated in FIG. 6C, including automatically moving the current position indicator 606 to the location of marker 608.

In some cases, it is desirable to pin a scan (e.g., to maintain display of a scan in image data user interface 602) when the input directed to the scrubber bar 604 is being detected. In some examples, while the input directed to the scrubber bar 604 is being detected, the electronic device 101 does not detect an input for pinning a scan of the plurality of scans. In response to not detecting the input for pinning the scan of the plurality of scans while the input directed to the scrubber bar 604 is being received, the electronic device 101 may automatically scrub through the plurality of scans to return the image data user interface 602 to displaying the scan that corresponds to the current pose of the camera 312 (e.g., the scan that corresponds to the position of marker 608) as described above. In some examples, while the input directed to the scrubber bar 604 is being detected, the electronic device 101 detects an input (e.g., a voice input or another type of input described herein) for pinning a scan of the plurality of scans. For example, the input for pinning the scan may include a voice input of the user 301 indicating (e.g., that includes the word or command) “pin” while a gaze of the user is directed at the image data user interface 602. For example, while the input directed to the scrubber bar 604 is being detected as shown in FIG. 6D, the electronic device 101 may detect the input for pinning the scan illustrated in image data user interface 602 in FIG. 6D. In response to detecting input for pinning the scan of the plurality of scans, the electronic device 101 may maintain display of the pinned scan such that were the input directed to the scrubber bar 604 in FIG. 6D to be ceased while the electronic device 101 is displaying the scan illustrated in FIG. 6D, the electronic device 101 would maintain display of the pinned scan in the image data user interface 602 instead of automatically scrubbing back to the scan that corresponds to the current pose of the camera 312. In some examples, when the scan is pinned, the electronic device 101 displays an indication (e.g., an icon or a user interface element such as a pin) in the image data user interface 602 notifying the user 301 of the electronic device 101 that the scan is pinned.

In some examples, while displaying the image data user interface 602, the electronic device 101 detects and responds to input requesting to annotate a scan of the plurality of scans by annotating the scan of the plurality of scans in accordance with the input, as shown in FIGS. 6B and 6E. For example, in FIG. 6B, the electronic device 101 may detect input 611 requesting annotation of a portion of the displayed scan in the image data user interface 602. For example, the input 611 may include a voice input of the user 301 while gaze of the user 301 and/or a hand of the user 301 is directed to a portion of the image data user interface 602, and/or may include other types of input described herein. In response, the electronic device 101 may annotate the portion of the displayed scan, as shown with portion 610 in FIG. 6E. For example, the electronic device 101 may have annotated portion 610 in FIG. 6E in response to the annotation input received in FIG. 6B. In some examples, the electronic device 101 saves the annotations made on scans of the plurality of scans such that were the electronic device 101 to scrub away from the annotated scan and then scrub back to the scan that was previously annotated, the electronic device 101 would display the scan as previously annotated.

FIG. 6G is a flow diagram illustrating a method 650 for updating display of user interfaces in response to detecting camera movement according to some examples of the disclosure. It is understood that method 650 is an example and that more, fewer, or different operations can be performed in the same or in a different order. Additionally, the operations in method 650 described below are, optionally, implemented by running one or more functional modules in an information processing apparatus such as general-purpose processors (e.g., as described with respect to FIG. 2) or application specific chips, and/or by other components of FIG. 2.

Therefore, according to the above, some examples of the disclosure are directed to a method (e.g., method 650 of FIG. 6G) including at an electronic device in communication with one or more displays and one or more input devices, including a camera, presenting (652), via the one or more displays, a view of a physical environment of the electronic device from a viewpoint of the electronic device in the physical environment, the view of the physical environment including an external view of a physical object, while presenting the view of the physical environment, and while a first location of the camera corresponds to a first location of the physical object, concurrently displaying (654), via the one or more displays, a first user interface including a video feed from the camera that is based on the camera having the first location, and a second user interface including first internal image data of the physical object of a plurality of image data of the physical object captured by a device different from the camera, while concurrently displaying the first user interface and the second user interface that is displayed while presenting the view of the physical environment, and while the first location of the camera corresponds to the first location of the physical object, detecting (656) movement of the camera from the first location corresponding to the first location of the physical object to a second location corresponding to a second location of the physical object, different from the first location corresponding to the first location of the physical object, and in response to detecting the movement of the camera from the first location to the second location, updating display (658) of the first user interface to include video feed from the camera based on the second location of the camera and the second user interface to include second internal image data of the plurality of image data of the physical object, different from the first internal image data.

Additionally or alternatively, in some examples, updating the second user interface includes displaying scrubbing through the plurality of image data of the physical object from the first internal image data to the second internal image data.

Additionally or alternatively, in some examples, updating display of the first user interface and updating display of the second user interface is performed concurrently.

Additionally or alternatively in some examples, the view of the physical environment includes a view of a portion of the camera.

Additionally or alternatively in some examples, the movement of the camera from the first location to the second location includes a change of depth of the camera relative to the physical object.

Additionally or alternatively, in some examples, method 650 includes displaying the second user interface including the first internal image data of the physical object and a scrubber bar, wherein the scrubber bar including a position indicator in the scrubber bar that is moved as the camera is moved.

Additionally or alternatively, in some examples, method 650 includes displaying the second user interface including the first internal image data of the physical object and a scrubber bar, wherein the scrubber bar includes a position indicator in the scrubber bar that is moved as the camera is moved, and after updating display of the first user interface and of the second user interface in response to detecting the movement of the camera from the first location to the second location, and while the camera has a respective location, detecting an input directed to the scrubber bar, and in response to detecting the input directed to the scrubber bar, scrubbing through the plurality of image data of the physical object in accordance with the input.

Additionally or alternatively, in some examples, method 650 includes while the camera has the respective location, in accordance with a determination that while scrubbing through the plurality of image data of the physical object, the second user interface shows respective internal image data of the physical object that is based on the camera having the respective location, forgoing scrubbing past the respective internal image data of the physical object that is based on the camera having the respective location, including maintaining display of the respective internal image data of the physical object that is based on the camera having the respective location.

Additionally or alternatively, in some examples, the camera is a laparoscopic camera, and wherein the plurality of image data of the physical object are Magnetic Resonance Imaging (MRI) scans of the physical object.

Additionally or alternatively, in some examples, method 650 includes while concurrently displaying the first user interface and the second user interface, displaying a user interface element that is selectable to display a model of an object and detecting input directed to the user interface element, and in response to detecting the input directed to the user interface element, maintaining display of the first user interface, ceasing display of the second user interface, and displaying, via the one or more displays, a third user interface including a first amount of the model of the object.

Additionally or alternatively, in some examples, method 650 includes while concurrently displaying the first user interface and the third user interface, detecting an input for modifying a view of the model of the object, and in response to detecting the input for modifying the view of the model of the second object, modifying the view of the model of the object, including displaying, via the one or more displays, a second amount of the model of the second object, different from the first amount of the model of the second object.

Additionally or alternatively, in some examples, detecting movement of the camera from the first location corresponding to the first location of the physical object to the second location corresponding to the second location of the physical object includes detecting user interaction with the camera.

Additionally or alternatively, in some examples, detecting movement of the camera from the first location corresponding to the first location of the physical object to the second location corresponding to the second location of the physical object includes detecting a change in depth of the camera relative to the physical environment.

Additionally or alternatively, in some examples, the camera is a laparoscopic camera, the physical object is a body of a patient.

Additionally or alternatively, in some examples, the device different from the camera is a Magnetic Resonance Imaging (MRI) device (e.g., an MRI system). Additionally or alternatively, in some examples, the device different from the camera is an X-ray system, a Computerized tomography (CT) system, or an Ultrasound system, or another type of device.

Additionally or alternatively, in some examples, the electronic device includes a head-mounted display system and the one or more input devices include one or more sensors configured to detect interaction with the camera.

Attention is now directed towards examples of an electronic device displaying live stereoscopic camera feed with special effects in accordance with some examples.

In some cases, it is desirable for camera 312 to be a stereoscopic camera so that depth effects may be shown in the live camera feed user interface 314. For example, the physical object 310 is optionally a body of a patient, and the camera 312 is optionally a stereo laparoscopic camera that is inside of the body and is being used to view an area of the inside of the body on which one or more operations will be performed in a surgical operation. A stereoscopic camera that captures images and presents them with an amount of stereo disparity may provide an enhanced spatial understanding of a spatial arrangement of elements of the area in the body (e.g., of organs, veins, arteries, and/or other body parts) and/or of the placement of medical instruments relative to the inside of the body.

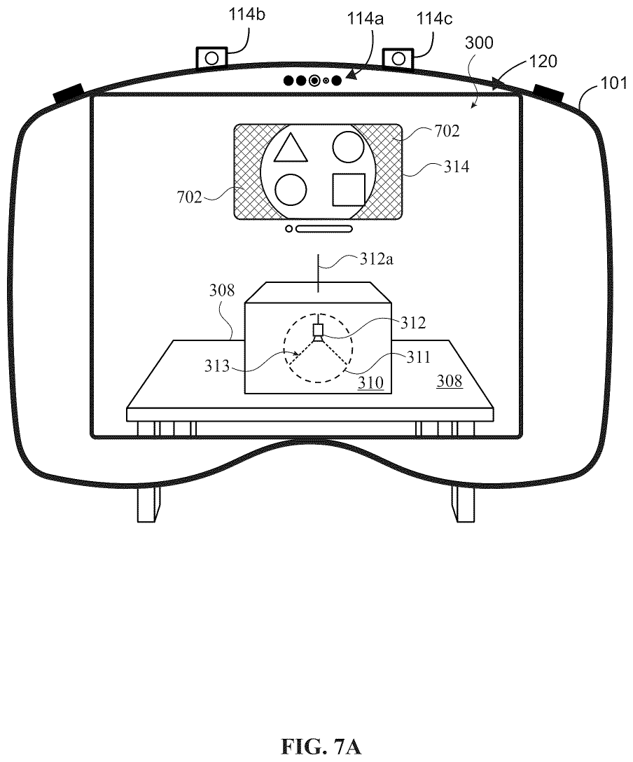

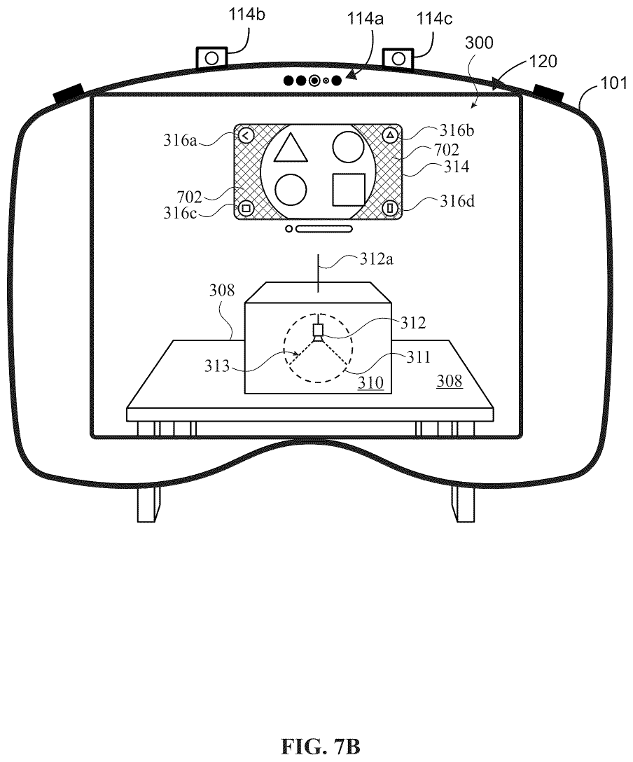

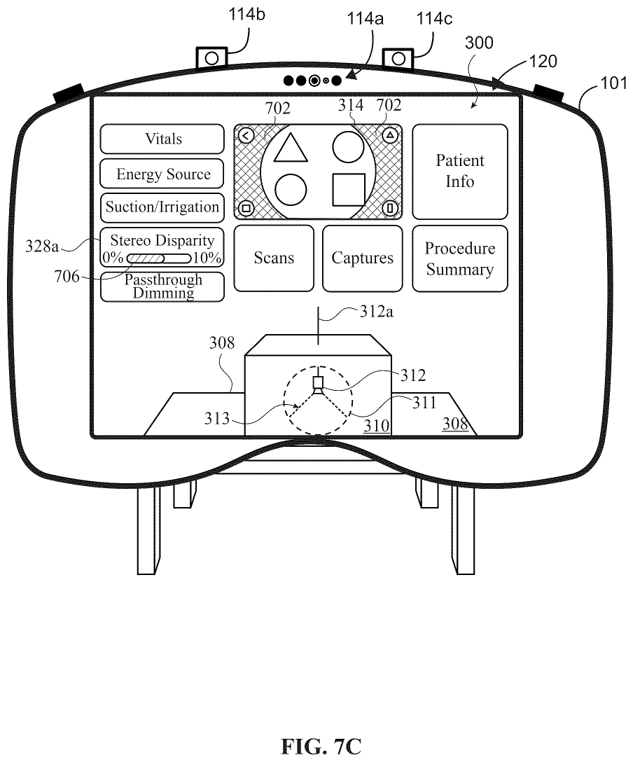

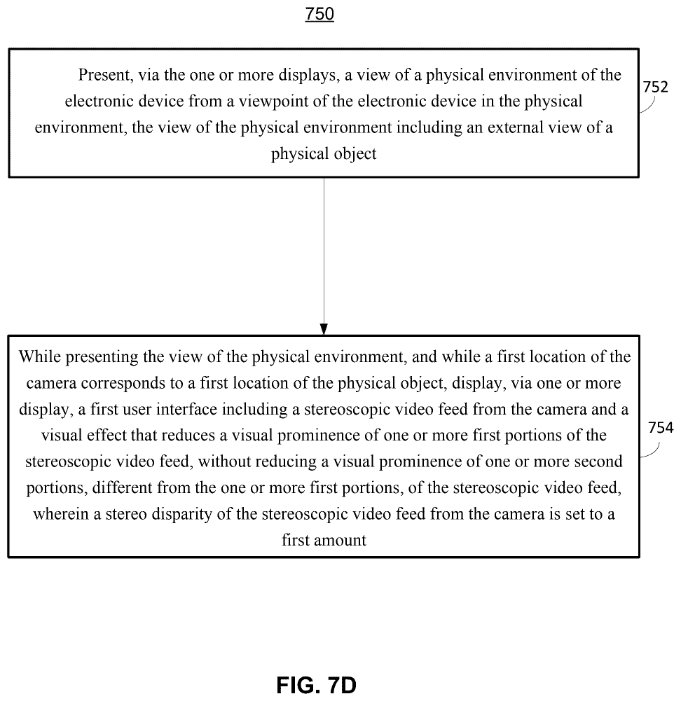

Note that in FIGS. 7A-7C, the electronic device 101 displays the view of the three-dimensional environment 300 visible through the display 120 from the viewpoint of the user 301 in the respective figure, and that the relative placements of live camera feed user interface 314, the physical object 310, the user 301, and the table 308 may be similar to (e.g., same as) the placements of these in FIG. 3A and/or 3H.

In some examples, camera 312 described herein is a stereoscopic camera. FIG. 7A illustrates live camera feed user interface 314 displaying feed from camera 312, which is a stereoscopic camera, in accordance with some examples. In the illustrated example of FIG. 7A, the electronic device 101 is streaming feed from the camera 312, and the feed is being displayed in live camera feed user interface 314.

In some examples, when the live camera feed user interface 314 is streaming stereo, the electronic device 101 displays a mask effect 702 in live camera feed user interface 314, such as shown in FIG. 7A. The mask effect 702 is not part of the feed that is from the camera 312, but is applied by the electronic device 101 to cover portions of the live camera feed user interface 314 that includes the feed. Note that the live camera feed user interface 314 is optionally showing the stereo feed from the camera 312 as captured by the camera 312 without the electronic device 101 having removed portions of the captured stereo feed. That is, in some examples, the live camera feed user interface 314, including the portions of the live camera feed user interface 314 that are covered by mask effect 702, includes the stereo feed from the camera 312. As such, in some examples, mask effect 702 is covering portions of the stereo feed that is captured by the camera 312. As another example, were the mask effect 702 removed from the live camera feed user interface 314, the live camera feed user interface 314 would optionally display the portion of the stereo feed that was being reduced in visual prominence by the mask effect 702 at the same visual prominence as the other portions of the stereo feed in the live camera feed user interface 314 that were not covered by the mask effect 702. In some examples, the electronic device 101 displays mask effect 702 to hide one or more artifacts that would be visible in the portions of the live camera feed user interface 314 were the mask effect 702 not displayed.

In the illustrated example of FIG. 7A, the mask effect 702 is covering left and right sides of the live camera feed user interface 314. In some examples, a visual prominence (e.g., a visual emphasis, a level of opacity, etc.) of the mask effect 702 at the boundary between the mask effect 702 and the portion of the live camera feed user interface 314 outside of the mask effect 702 is less than a visual prominence of the mask effect 702 at the edges of the live camera feed user interface 314 that have the mask effect 702 applied. As such, in the live camera feed user interface 314, the further away from the above-described boundary, the greater the visual prominence of the mask effect 702 and the lesser the visual prominence the camera feed in the live camera feed user interface 314 that is being covered by the mask effect 702. In some examples, the electronic device 101 displays mask effect 702 to increase a visual differentiation between the live camera feed user interface 314, which has depth effects applied, and other portions of the user's environment that are visible via electronic device 101. For example, when live camera feed user interface 314 is a stream of stereo video feed, the video feed is enhanced with depth effects, but the same enhancement is not being applied outside of the live camera feed user interface 314 (e.g., outside of the live camera feed user interface 314 the electronic device may be presenting (e.g., via optical or video passthrough) a view of the physical environment of the user of the electronic device 101). To increase a level of spatial understanding between the stream of stereo video feed, which has stereo effects applied, and the portions of the three-dimensional environment that are presented outside of the live camera feed user interface 314, the electronic device 101 may display the mask effect 702, such as illustrated in FIG. 7A. Doing so may reduce errors when interacting with the electronic device 101.

In some examples, the electronic device 101 displays mask effect 702 to provide for increased spatial understanding between the stereo feed in live camera feed user interface 314, which has the depth effects applied, and user interface elements that may be displayed in the live camera feed user interface 314, such as the user interface elements 316a-316d in FIG. 7B. For example, in FIG. 7B, the electronic device 101 displays the user interface elements 316a-316d at the locations of mask effect 702. In some examples, by displaying the user interface element 316a-316d at the location of the mask effect 702 in the live camera feed user interface 314, the placements of the user interface element 316a-316d are more easily determinable by the user 301 of the electronic device 101. For example, were the mask effect 702 not displayed and were the user interface element 316a-316d displayed in the live camera feed user interface 314 that is streaming stereo feed, misunderstanding between the placements of the user interface elements may arise since they would overlap portions of the live camera feed user interface 314 that has depth effects applied. In some examples, user interface elements 716a-716d fade out (e.g., cease to be displayed) after selection of any of user interface elements 716a-716d. By displaying the user interface element 316a-316d at the location of the mask effect 702 in the live camera feed user interface 314, errors resulting from interaction with the electronic device 101 may be reduced.

In some examples, the electronic device 101 displays the user interface elements 316a-316d in FIG. 7B in response to input detected while the electronic device 101 is displaying live camera feed user interface 314 of FIG. 7A. For example, while displaying live camera feed user interface 314 of FIG. 7A, the electronic device 101 may detect input (e.g., gaze of the user, input from the hand of the user (e.g., the hand of the user being in a pinch pose while gaze of the user is directed to the live camera feed user interface 314), voice input, or another type of input) requesting display of the user interface elements 316a-316d. In response, the electronic device 101 may display the live camera feed user interface 314 as shown in FIG. 7B with the user interface elements 316a-316d. User interface elements 316a-316d are selectable to perform corresponding operations as previously described with reference to FIG. 3A.

In some examples, live camera feed user interface 314a in the widget dashboard user interface 330 (e.g., of FIG. 3H) is a stream from a stereoscopic camera. In some examples, when displaying live camera feed user interface 314a in the widget dashboard user interface 330 and streaming stereo feed, the electronic device 101 may display mask effect 702, such as shown in FIG. 7C. In some examples, the electronic device 101 maintains the set amount of stereo when transitioning between display of the live camera feed user interface 314a in the widget dashboard user interface 330 (e.g., live camera feed user interface 314a in the widget dashboard user interface 330 in FIG. 3H) and display of the live camera feed user interface 314 in FIG. 7A. For example, when the live camera feed user interface 314a in the widget dashboard user interface 330 in FIG. 7C is displayed, the amount of stereo disparity is set to a first amount, and in response to detecting an input requesting transition from display of the widget dashboard user interface 330 in FIG. 7C to display of live camera feed user interface 314 in FIG. 7A, the electronic device 101 may transition from display of the widget dashboard user interface 330 in FIG. 7C to display of live camera feed user interface 314 in FIG. 7A while maintaining display of the stereo feed with the stereo disparity set the first amount. Continuing with this example, when the live camera feed user interface 314 is displayed in response to the input described above, the electronic device 101 may display the live camera feed user interface 314 with the stereo disparity being the same amount as in FIG. 7C. As another example, when the live camera feed user interface 314 in FIG. 7A is displayed, the amount of stereo disparity is set to a particular amount, and in response to detecting a transition from display of the live camera feed user interface 314 in FIG. 7A to display of widget dashboard user interface 330 in FIG. 7C, the electronic device 101 may transition from display of live camera feed user interface 314 in FIG. 7A to display of widget dashboard user interface 330 in FIG. 7C while maintaining display of the camera feed with the stereo disparity set to the particular amount. Continuing with this example, when the live camera feed user interface 314a of FIG. 7C is displayed in response to the input described above, the electronic device 101 may display the live camera feed user interface 314a with the stereo disparity being the same amount as in FIG. 7A. In some examples, live camera feed user interface 314a of FIG. 7C is of a first size and live camera feed user interface 314 of FIG. 7A is of a second size greater than the first size, and when transitioning between the first size and the second size, the amount of stereo disparity is maintained.

In some examples, the electronic device 101 detects and responds to input for re-sizing the live camera feed user interface 314 of FIG. 7A. For example, while displaying the live camera feed user interface 314 of FIG. 7A, the electronic device 101 may detect input (e.g., gaze, voice, input involving a hand, and/or another type of input) from the user requesting to change a size of the live camera feed user interface 314 from a first size to a second size different from (e.g., greater than or less than) the first size. In response, the electronic device 101 may change the size of the live camera feed user interface 314 from the first size to the second size while maintaining the same amount of stereo disparity (e.g., the amount of stereo disparity is optionally the same at the second size as the first size). As such, the electronic device 101 optionally provides for re-sizing the stereoscopic feed shown in the live camera feed user interface 314 while maintaining the same amount of stereo disparity.

In some examples, the electronic device 101 changes the amount of disparity in the live camera feed user interface 314 in response to input requesting the change. For example, while displaying widget dashboard user interface 330 of FIG. 7C, the electronic device 101 may detect an input directed to stereo disparity widget 328d requesting change in an amount of stereo disparity. In the illustrated example of FIG. 7C, stereo disparity widget 328d includes a slider 706 for setting an amount of stereo disparity. In response to detecting input (e.g., gaze of the user, voice input from the user, input from the hand of the user, or another type of input) directed to slider 706, the electronic device 101 may change the amount of stereo disparity in accordance with the input. That is, the electronic device 101 may cause the camera 312 to capture images according to the second amount of stereo disparity, cause the widget dashboard user interface 330 of FIG. 7C to update display of the live camera feed user interface 314a to have the second amount of stereo disparity applied, and/or cause the position of the slider 706 to update to reflect the set amount of stereo disparity being the second amount. In addition, were the electronic device 101 to display live camera feed user interface 314 after detecting the input to change the amount of stereo disparity, the electronic device 101 would update the live camera feed user interface 314 to display the stereo feed according to the second amount of stereo disparity. Further, if when the input is detected, the amount of stereo disparity is set to a first amount, and the input requests change in the amount of stereo disparity to a second amount, different than the first amount, then the electronic device 101 may change the amount of stereo disparity to the second amount in accordance with the input. Were the second amount less than the first amount of stereo disparity, the electronic device 101 would reduce the amount of stereo disparity with which the feed in the live camera feed user interface 314 is presented and where the second amount greater than the first amount of stereo disparity, the electronic device 101 would increase the amount of stereo disparity with which the feed in the live camera feed user interface 314 is presented.

Note that, in some examples, the input that requests change in the amount of stereo disparity may be an input requesting a setting of the amount of stereo disparity to a maximum amount of stereo disparity. Also, note that, in some examples, the input that requests change in the amount of stereo disparity may be an input requesting a setting of the amount of stereo disparity to a minimum amount of stereo disparity, which may correspond to specifying a minimum amount of stereo disparity or no stereo disparity at all.

In some examples, the electronic device 101 toggles a stereo disparity mode without detecting input directed to the stereo disparity widget 328d. In some examples, camera 312 described herein can operate as a stereoscopic camera or as a camera with no stereo disparity active. In some examples, the electronic device 101 changes the stereo disparity setting based on an amount of relative movement between the camera 312 and the physical object 310. For example, if the electronic device 101 were to detect that the camera 312 in FIG. 7A is moving in the physical environment beyond a threshold amount of movement (or were to detect that relative movement between the camera 312 and the physical object 310 in the field of view 313 of the camera 312 is beyond a threshold amount of movement, such as if the physical object 310 in the field of view 313 of the camera 312 is moving as shown in the live camera feed user interface 314 beyond the threshold amount of movement), the electronic device 101 may automatically change (e.g., reduce) an amount of stereo disparity, such as reduce the amount of stereo disparity to no stereo disparity. For example, live camera feed user interface 314 would include video feed from the camera 312 that may not have stereo in response to detecting the movement that is beyond the threshold amount of movement. Continuing with this example, if, after detecting that the camera 312 is moving in the physical environment beyond the threshold amount of movement, the electronic device 101 detects that the camera 312 is no longer moving in the physical environment beyond the threshold amount of movement, the electronic device 101 may automatically change (e.g., increase) the amount of stereo disparity to the same amount it was before it was detected that the camera 312 was moving beyond the threshold amount of movement, or may maintain the camera 312 having the reduced amount of stereo disparity until the user provides input requesting a change in the amount of stereo disparity.

In some examples, the electronic device 101 displays an indication (e.g., a user interface element, textual content, etc.) of a suggested distance between the camera 312 and the portion of the physical object 310 that is in the field of view 313 of the camera 312. For example, the stereoscopic functionalities of the camera 312 have optimal performance when the portion of the physical object 310 is at or beyond the suggested distance (e.g., 3 cm, 5 cm, 10 cm, 20 cm, or another suggested distance) from the camera 312. In some examples, the electronic device 101 displays the indication when a distance between the camera 312 and the portion of the physical object 310 that is in the field of view 313 of the camera 312 is not within a threshold of the suggested distance. In some examples, the electronic device 101 forgoes displaying the indication when a distance between the camera 312 and the portion of the physical object 310 that is in the field of view 313 of the camera 312 is not within a threshold of the suggested distance. In some examples, the electronic device 101 suggests different distances between the camera 312 and the portion of the physical object 310 that is in the field of view 313 of the camera 312 based on the amount of stereo disparity that is desired. For example, if the amount of stereo disparity is set to a first amount (e.g., via the slider 706), then the electronic device 101 may display a suggested distance between the camera 312 and the portion of the physical object 310 that is in the field of view 313 of the camera 312 as being a first distance, and if the amount of stereo disparity is set to a second amount, different from the first amount, then the electronic device 101 may display a suggested distance between the camera 312 and the portion of the physical object 310 that is in the field of view 313 of the camera 312 as being a second distance that is different from the first distance. In some examples, the electronic device 101 suggests the same distance between the camera 312 and the portion of the physical object 310 that is in the field of view 313 of the camera 312 independent of the amount of stereo disparity (e.g., while stereo video is being streamed, the suggested distance is constant with respect to the amount of stereo disparity).

FIG. 7D is a flow diagram illustrating a method 750 for displaying live stereoscopic camera feed with special effects according to some examples of the disclosure. It is understood that method 750 is an example and that more, fewer, or different operations can be performed in the same or in a different order. Additionally, the operations in method 750 described below are, optionally, implemented by running one or more functional modules in an information processing apparatus such as general-purpose processors (e.g., as described with respect to FIG. 2) or application specific chips, and/or by other components of FIG. 2.

Therefore, according to the above, some examples of the disclosure are directed to a method (e.g., method 750 of FIG. 7D) including at an electronic device in communication with one or more displays and one or more input devices, including a camera, presenting (752), via the one or more displays, a view of a physical environment of the electronic device from a viewpoint of the electronic device in the physical environment, the view of the physical environment including an external view of a physical object, while presenting the view of the physical environment, and while a first location of the camera corresponds to a first location of the physical object, displaying (754), via the one or more displays a first user interface including a stereoscopic video feed from the camera and a visual effect that reduces a visual prominence of one or more first portions of the stereoscopic video feed, without reducing a visual prominence of one or more second portions, different from the one or more first portions, of the stereoscopic video feed, wherein a stereo disparity of the stereoscopic video feed from the camera is set to a first amount.

Additionally or alternatively, in some examples, the visual effect is a masking effect applied to the one or more first portions of the stereoscopic video feed.

Additionally or alternatively, in some examples, the one or more first portions of the stereoscopic video feed include one or more edge regions of the stereoscopic video feed.

Additionally or alternatively, in some examples, the one or more first portions of the stereoscopic video feed include one or more edges of the stereoscopic video feed in the first user interface, and wherein the one or more second portions include one or more central portions of the stereoscopic video feed in the first user interface.

Additionally or alternatively, in some examples, the stereoscopic video feed is a live stream of stereo video feed. Additionally or alternatively, in some examples, the live stream is transmitted to the electronic device via a wireless connection.

Additionally or alternatively, in some examples, method 750 includes displaying, via the one or more displays, a user interface element that indicates a distance between the camera and a portion of the physical object that is shown in the stereoscopic video feed.

Additionally or alternatively, in some examples, method 750 includes displaying, via the one or more displays, a user interface element selectable to change an amount of stereo disparity with which the stereoscopic video feed from the camera is displayed, while displaying the user interface element, and while the stereo disparity of the stereoscopic video feed from the camera is set to the first amount, detecting, via the one or more input devices, input directed to the user interface element, the input corresponding to a request change of the amount of stereo disparity from the first amount to a second amount, different from the first amount, and in response to the input changing the amount of stereo disparity from the first amount to the second amount and displaying, via the one or more displays, the stereoscopic video feed having the second amount of stereo disparity.

Additionally or alternatively, in some examples, method 750 includes displaying, via the one or more displays, one or more respective user interface elements that are selectable to perform one or more respective operations associated with the first user interface, wherein the one or more respective user interface elements are displayed in the first user interface at one or more locations corresponding to the one or more first portions of the stereoscopic video feed. Additionally or alternatively, in some examples, method 750 includes while displaying the first user interface without the one more respective user interface elements, detecting, via the one or more input devices, input requesting display of the one or more respective user interface elements, and in response to detecting the input requesting display of the one or more respective user interface elements, displaying, via the one or more displays, the first user interface including the one or more respective user interface elements that are selectable to perform the one or more respective operations.

Additionally or alternatively, in some examples, method 750 includes while displaying the first user interface including the stereoscopic video feed from the camera, detecting, via the one or more input devices, movement of the camera, and in response to detecting the movement of the camera in accordance with a determination that the movement of the camera is less than a threshold amount of movement, maintaining display of the stereoscopic video feed from the camera, and in accordance with a determination that the movement of the camera is greater than the threshold amount of movement, update display of the first user interface to include video feed from the camera that is different from stereoscopic video feed from the camera. For example, the video feed from the camera that is different from stereoscopic video feed may be video feed that is not stereoscopic.

Additionally or alternatively, in some examples, method 750 includes while displaying the first user interface including the stereoscopic video feed from the camera, detecting, via the one or more input devices, an input requesting a re-sizing of the stereoscopic video feed in the first user interface, and in response to detecting the input, re-sizing the stereoscopic video feed in the first user interface in accordance with the input.

Additionally or alternatively, in some examples, the camera is laparoscopic stereo camera.

Additionally or alternatively, in some examples, the one or more displays are part of a head-mounted display system.

Attention is now directed to an electronic device detecting and responding to inputs for annotating objects in the camera feed in accordance with some examples of the disclosure.

In some cases, it is desirable to annotate portions of the physical object that are shown in the live camera feed user interface 314. For example, the live camera feed user interface 314 may be showing a view of a uterus of a patient, and a surgeon may desire to virtually annotate a portion of the uterus to assist in one or more operations that are to be performed on the uterus. As another example, the surgeon may desire to virtually annotate a portion of an organ of a patient that is shown in the live camera feed user interface 314 for training purposes and/or as a reference in future operations involving the same organ in other patients.

In some examples, the electronic device 101 detects and responds to inputs for annotating portions of objects in the camera feed by virtually annotating the portions. In some examples, the annotations include annotations indicative of a point of interest in the physical object 310, a danger zone in the physical object 310, and/or a distance in the physical object 310, among other possibilities. In some examples, the input includes a voice input, gaze input, input from one or more hands of the user, and/or another type of input that requests annotation. In some examples, the input includes a request for annotation using a physical tool that is shown in the camera feed.

In some examples, when a portion of the physical object is annotated, the electronic device 101 locks the virtual annotation to the portion to maintain a spatial arrangement between the portion and the annotation such that the virtual annotation may move if relative movement between the camera and the portion were detected. In some examples, the electronic device 101 locks the virtual annotation to a portion of the physical object such that, were the portion to move (e.g., move in the physical environment relative to the camera), the virtual annotation would move in accordance with the movement of the portion. In some examples, the electronic device 101 locks the virtual annotation to the portion of the physical object such that were the camera to move while the object has not moved, the electronic device 101 would maintain the spatial arrangement of the virtual annotation relative to the portion rather than relative to the camera.

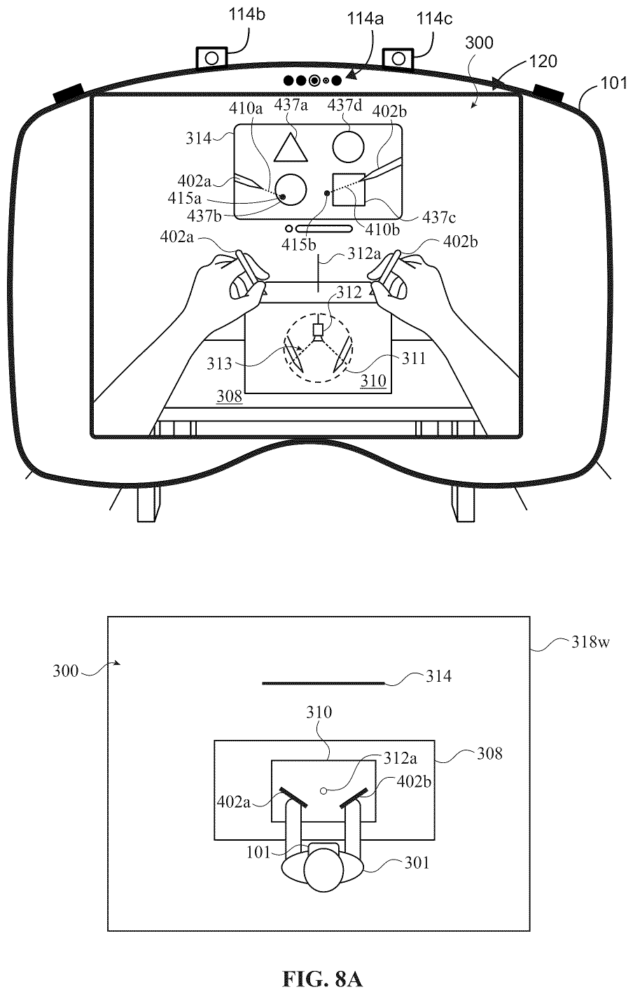

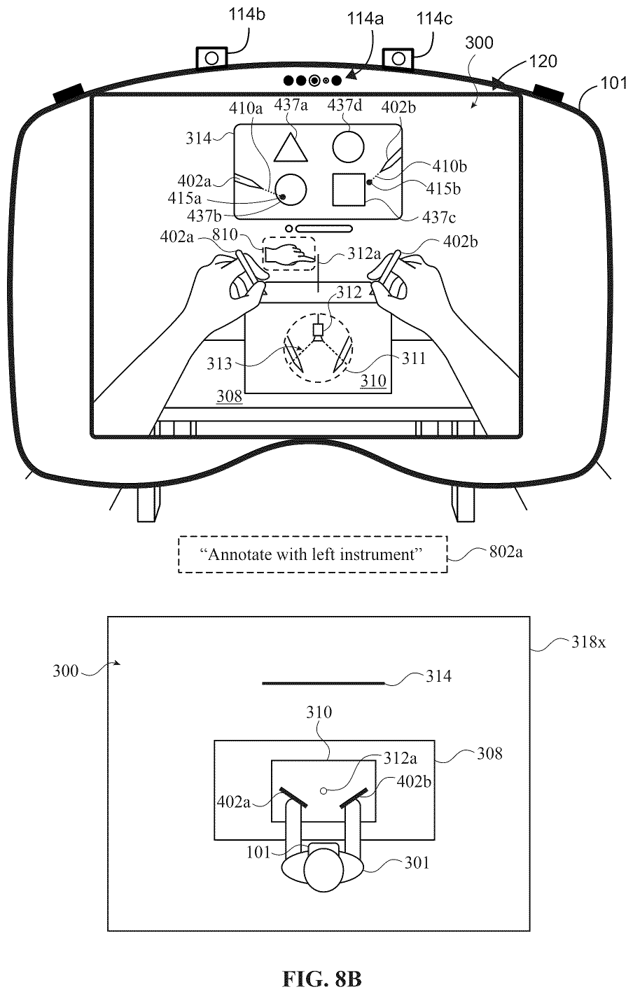

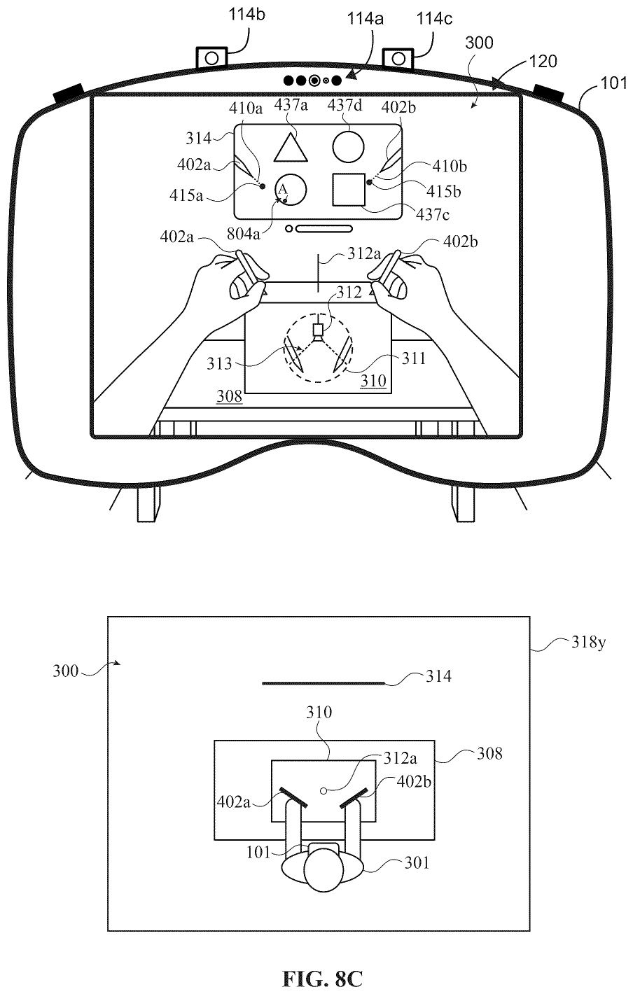

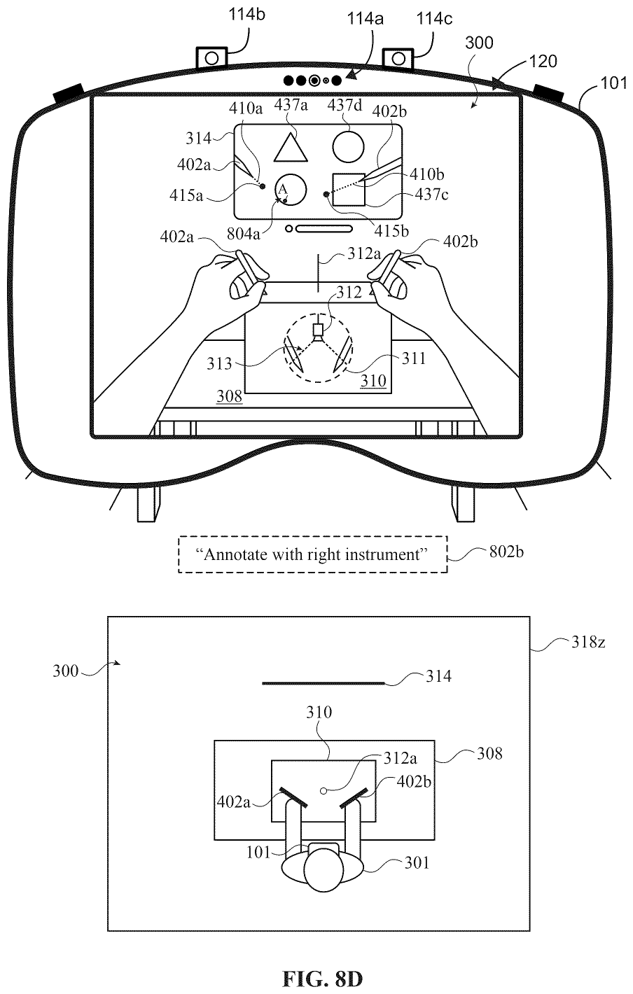

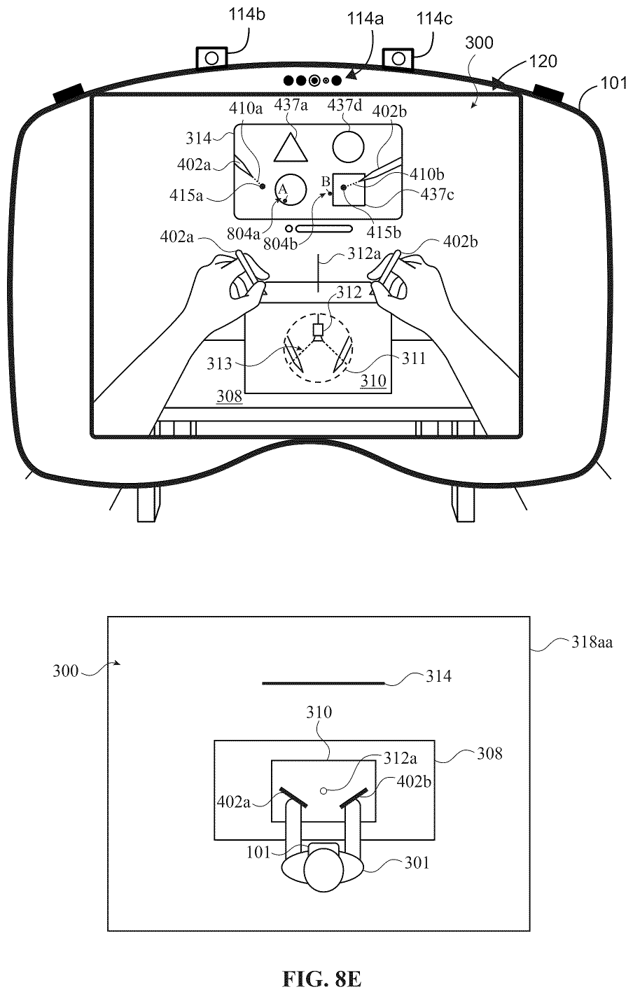

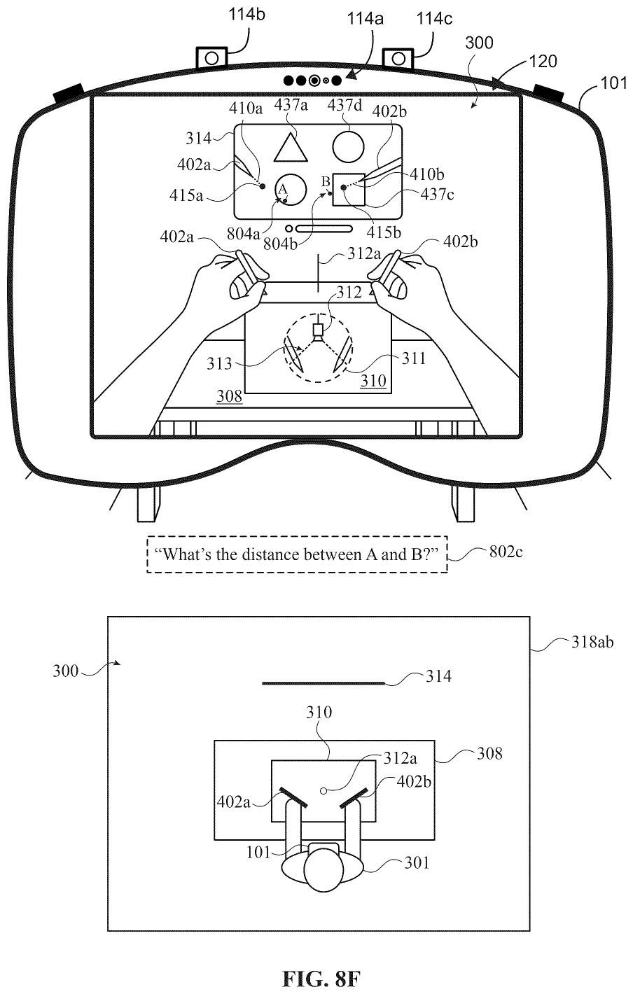

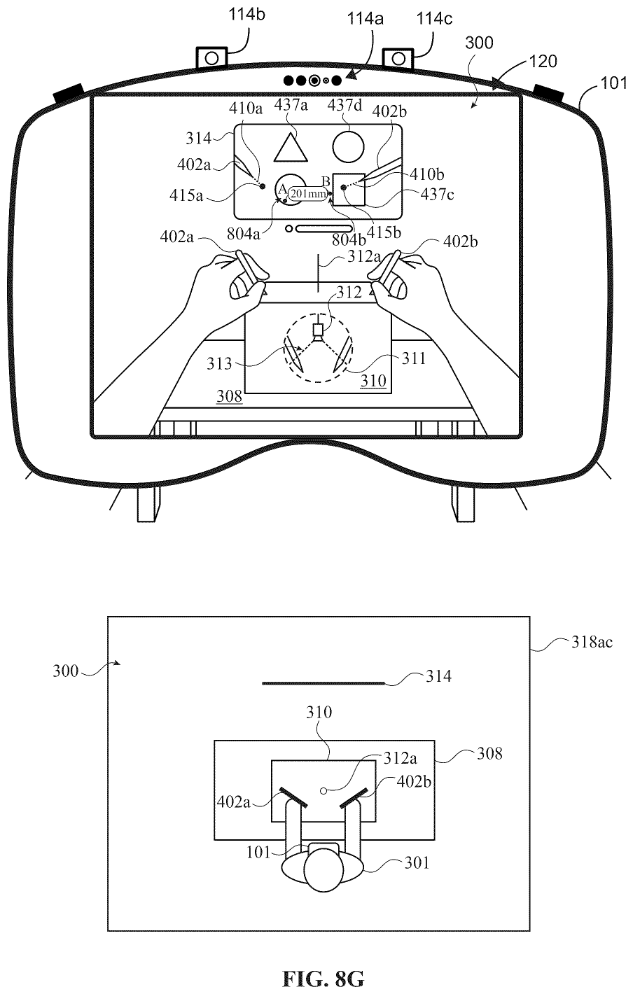

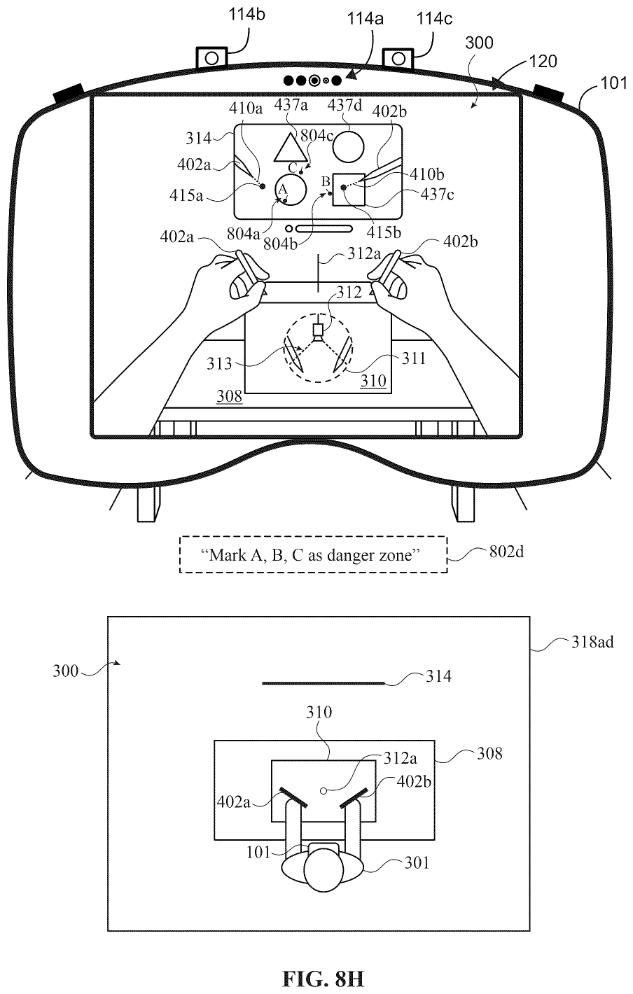

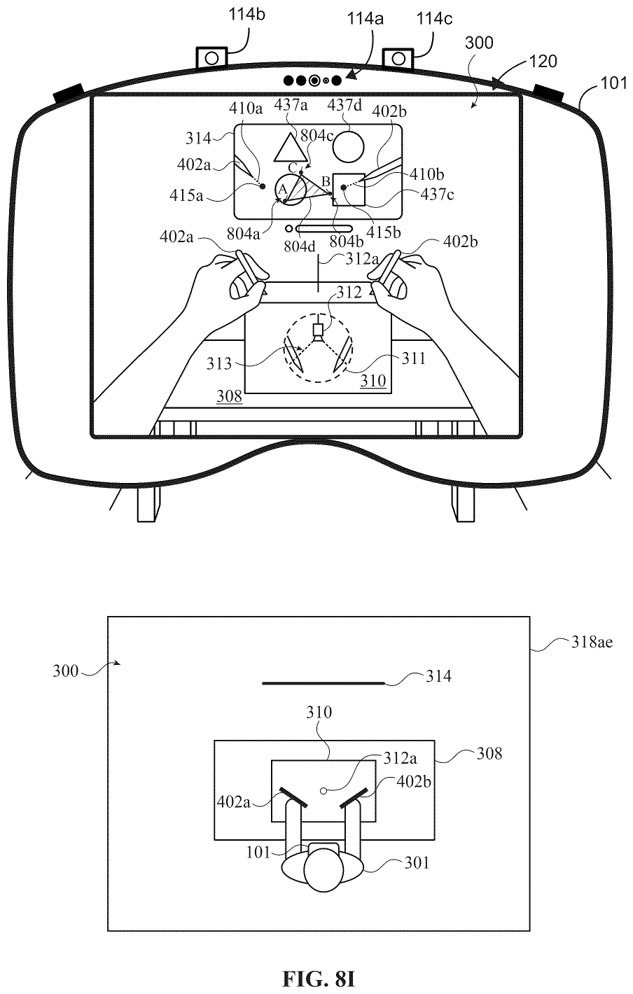

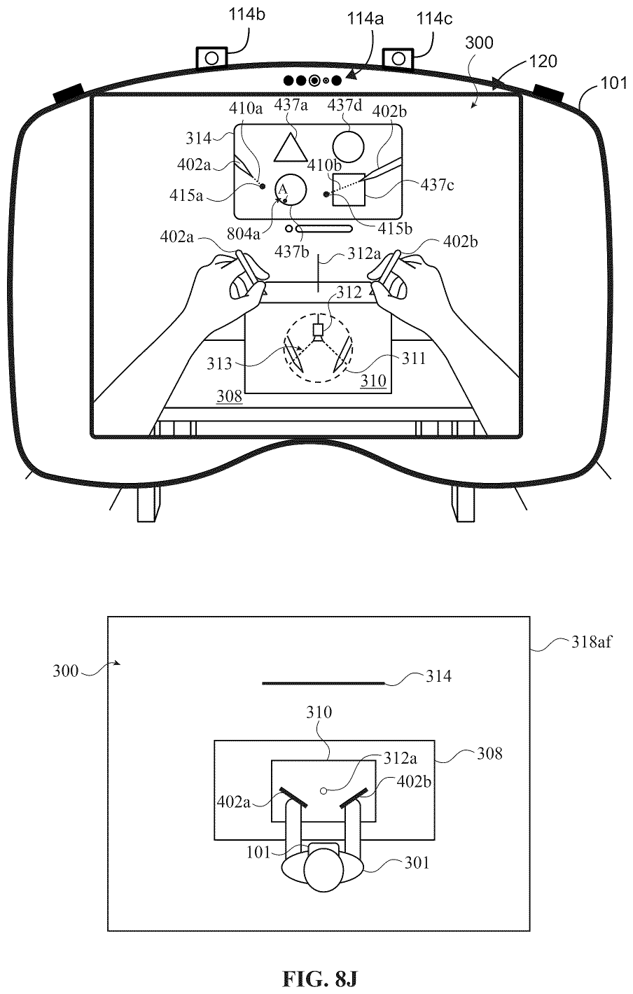

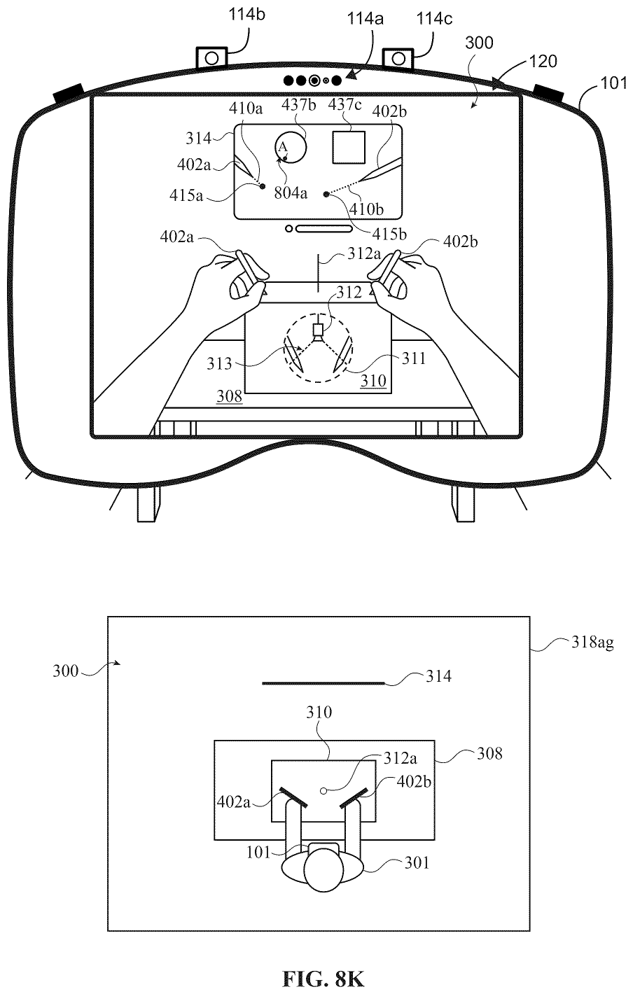

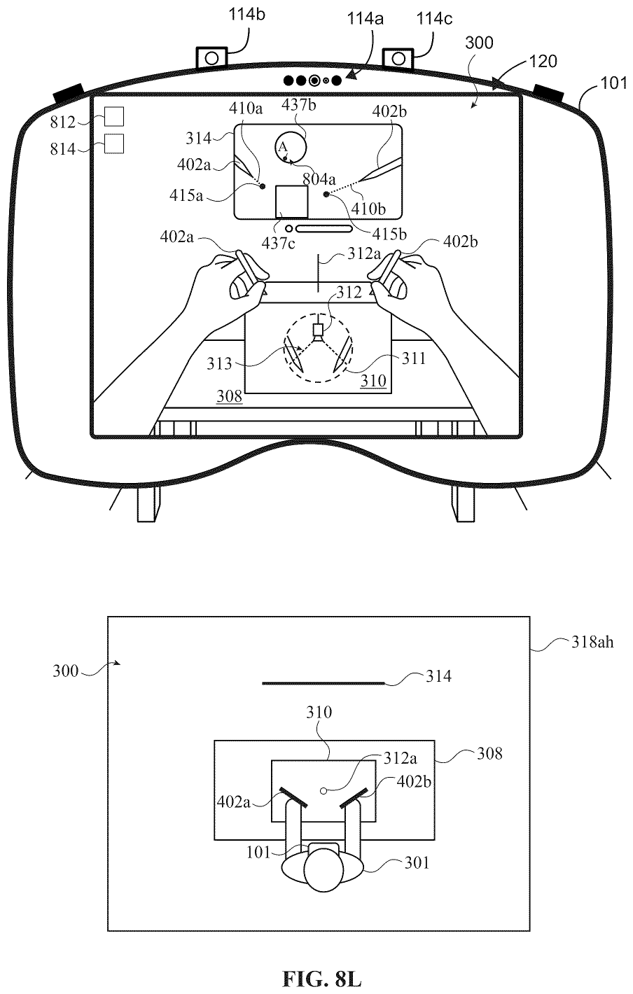

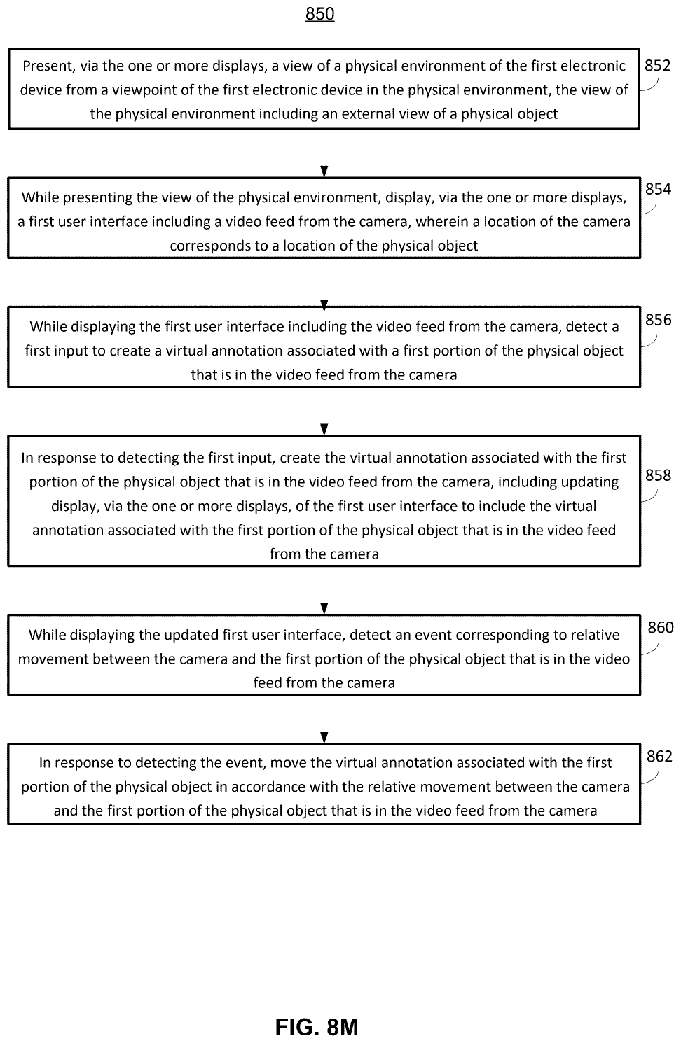

FIGS. 8A-8L illustrate examples of an electronic device presenting a live camera feed user interface 314 including video feed from camera 312 from inside the physical object 310, and virtually annotating in the live camera feed user interface 314.

For the purpose of illustration, FIGS. 8A-8L include respective top-down views 318w-318ah of the three-dimensional environment 300 that indicate the positions of various objects (e.g., real and/or virtual objects) in the three-dimensional environment 300 in a horizontal dimension and a depth dimension. The top-down view of the three-dimensional environment 300 further includes an indication of the viewpoint of the user 301 of the electronic device 101. For example, in FIG. 8A, the electronic device 101 displays the view of the three-dimensional environment 300 visible through the display 120 from the viewpoint of the user 301 illustrated in the top-down view 318w of the three-dimensional environment 300.

In the illustrated example of FIG. 8A, the field of view 313 of the camera 312 includes physical tool 402a (e.g., a first medical instrument), physical tool 402b (e.g., a second medical instrument), and surfaces 437a-437d. In some examples, physical object 310 is a body of a person, and surfaces 437a-437d corresponds to different organs in the body and/or are portions of organs in the body that are in the field of view 313 of the camera 312. In some examples, surfaces 437a-437d are portions of the same object (e.g., organ). In some examples, surfaces 437a-437d corresponds to specific surface areas of the same object (e.g., organ) in physical object 310, or to specific surface areas within physical object 310 generally. Note that objects 437a-437d are representative and nonlimiting. Also, note that the portion of the live camera feed user interface 314 in FIG. 8A that is outside of the surfaces 437a-437d and outside of physical tools 402a/402b are part of (e.g., comprise one or more internal surfaces of) the physical object 310 that is captured in the camera feed. In FIG. 8A, user 301 is holding physical tools 402a/402b (e.g., physical tool 402a is in the left hand of the user and physical tool 402b is in the right hand of the user), and as described previously with reference to FIG. 4C, the electronic device 101 displays a pointer 410a (e.g., first virtual pointer extending from a tip of the physical tool 402a to a position in the physical object 310, and including visual indication 415a on the position) and a pointer 410b (e.g., a second virtual pointer extending from a tip of the physical tool 402b to a position in the physical object 310 and including visual indication 415b on the position). In the illustrated example of FIG. 8A, pointer 410a is pointing towards a first position in the physical object 310, including being displayed on the first position (e.g., via visual indication 415a), and pointer 410b is pointing towards a second position in the physical object 310, including being displayed on the second position (e.g., via visual indication 415b). In FIG. 8A, the first position is the illustrated position on the object 437b.

FIG. 8B illustrates the electronic device 101 detecting an input requesting an annotation with physical tool 402a while the visual indication 415a of the pointer 410a is at the first position inside the physical object 310, as described with reference to FIG. 8A. In the illustrated example, the input includes an audio input 802a from the user 301 requesting that the electronic device 101 “annotate with left instrument”. It should be noted that other input types, including other hands-off input mechanisms or hand-on input mechanisms, and/or other input mechanism described herein, are contemplated. For example, the input of FIG. 8B additionally or alternatively includes input from a hand 810. For example, the electronic device 101 detects that camera part 312a is being tapped (e.g., contacted) by hand 810. In some examples, the hand is hand 301b of the user 301 of the electronic device 101. In some examples, the hand is a hand of someone other than the user 301 (and other than the physical object 310 were physical object 310 to include a hand). In some examples, the electronic device 101 detects a hand gesture without detecting contact between the camera 312 and the hand 810 associated with the input. For example, the electronic device 101 may detect the hand 810 being in a predetermined pose or the hand 810 performing a predetermined gesture (e.g., making a tapping gesture as if tapping a point in space) that the electronic device 101 interprets as input requesting annotation at the location of the pointer 410a. As such, the electronic device 101 can respond to annotation inputs detected using different mechanisms.

In response to the input in FIG. 8B, the electronic device 101 annotates the first position in physical object 310 on which the visual indication 415a was displayed, as shown with the first annotation 804a in FIG. 8C.

As shown in FIG. 8C, the electronic device 101 displays the first annotation 804a at the first position described with reference to FIG. 8A. Further, in FIG. 8C, though the visual indication 415a of the pointer 410a has moved from the first position to a third position, the electronic device 101 maintains the first annotation at the first position. Furthermore, in FIG. 8C, though pointer 410b has moved away from the second position described with reference to FIG. 8A, the electronic device 101 is not displaying an annotation at the second position because no input requesting annotation of the second position has been received.

FIG. 8D illustrates the electronic device 101 detecting an input requesting an annotation with physical tool 402b while the visual indication 415b of the pointer 410b is at the second position inside the physical object 310, as described with reference to FIG. 8A, and after detecting and responding to the input requesting annotation with the physical tool 402a described with reference to FIGS. 8B and 8C. In the illustrated example, the input includes an audio input 802b from the user 301 requesting that the electronic device 101 “annotate with right instrument”. It should be noted that other input mechanisms, including other hands-off input mechanisms or hand-on input mechanisms, are contemplated. In response to the input in FIG. 8D, the electronic device 101 annotates the second position in the body, as shown with the second annotation 804b in FIG. 8E.

In FIG. 8E, the electronic device 101 displays the second annotation 804b at the second position described with reference to FIG. 8A. Further, in FIG. 8E, though the visual indication 415b of the second pointer 410b has moved from the second position to a fourth position (e.g., due to movement of physical tool 402b), the electronic device 101 maintains the second annotation 804b at the second position.

Additionally, FIG. 8E illustrates the electronic device 101 concurrently displaying the first annotation 804a and the second annotation 804b. In the illustrated example of FIG. 8E, the first annotation 804a includes a first textual representation indicating “A” and a first pin, and the second annotation 804b includes a second textual representation indication “B” and a second pin. In some examples, the first pin is at the first position described with reference to FIG. 8A and the second pin is at the second position described with reference to FIG. 8A. In some examples, first textual representation is at the first position described with reference to FIG. 8A and the second textual representation is at the second position described with reference to FIG. 8A.

FIG. 8F illustrates the electronic device 101 detecting a request to indicate a distance between the first annotation 804a and the second annotation 804b. In the illustrated example of FIG. 8F, the input includes an audio input 802c from the user 301 that asks of the electronic device “what's the distance between “A” and “B”?”, to which the electronic device 101 interprets as a request to indicate the distance between the first annotation 804a and the second annotation 804b. It should be noted that other input mechanism, including other hands-off input mechanisms or hands-on input mechanisms, are contemplated. In response to the input in FIG. 8F, the electronic device 101 displays a notification 806, which indicates the distance between the first annotation 804a and the second annotation 804b, as shown in FIG. 8G. Note that from FIG. 8F to FIG. 8G, the orientations of the pins of the first annotation 804a and of the second annotation 804b have aligned with a hypothetical line extending from the first annotation 804a to the second annotation 804b. As such, in some examples, the electronic device 101 changes the orientations of the pins of the annotations to align them with the hypothetical line when a distance between them is requested. Additionally or alternatively, in some examples, the electronic device presents the distance via audio output. Additionally or alternatively, in some examples, the electronic device 101 displays two notifications of the distance—one in between the location of the first annotation 804a and the location of the second annotation 804b and another that is outside of the live camera feed user interface 314 where both notifications indicate the same distance since both are displayed in response to the input of FIG. 8F.

In some examples, the electronic device 101 detects and responds to a request to indicate a distance between the pointer 410a and the pointer 410b (e.g., the distance between the visual indication 415a and the visual indication 415b) by presenting one or more notifications of said distance in a similar manner that the distance between the first annotation 804a and the second annotation 804b was presented. For example, where the distance between the pointer 410a and the pointer 410b is a first distance when the input is request is detected, the electronic device 101 would present (e.g., display) an indication of that first distance, and were the distance a second distance, different from the first distance, the electronic device 101 would present an indication of that second distance.

FIG. 8H illustrates the electronic device 101 detecting a request to mark a zone (e.g., a danger zone or other predefined zone) in the physical object 310. In FIG. 8H, the electronic device 101 displays the first annotation 804a, second annotation 804b, and a third annotation 804c applied on respective portions inside of the physical object 310 (e.g., the first position, the second position, and a third position). In the illustrated example, the input includes an audio input 802d from the user 301 that requests that the electronic device 101 “Mark A, B, C as danger zone”, to which the electronic device 101 interprets as a request to mark the area defined by (e.g., bounded by) the first annotation 804a, second annotation 804b, and the third annotation 804c as a danger zone. It should be noted that other input mechanisms, including other hands-off input mechanisms or hand-on input mechanisms, are contemplated. In response to the input in FIG. 8H, the electronic device 101 marks the area between the first annotation 804a, second annotation 804b, and the third annotation 804c, as shown in FIG. 8I.

FIG. 8I illustrates the electronic device 101 responding to the input of FIG. 8H with display of fourth annotation 804d, which is an annotation covering the area (e.g., the surface area) defined by the first annotation 804a, second annotation 804b, and the third annotation 804c in the live camera feed user interface 314. Thus, in some examples, the electronic device 101 can detect and respond to input for annotating zones or areas inside the physical object 310, such as shown and described with reference to FIGS. 8H and 8I.

FIG. 8J illustrates an example of the electronic device 101 detecting and responding to an event corresponding to relative movement between the camera 312 and the first portion of the physical object 310 in accordance with some examples.

In some cases, a surface of the physical object 310 that is displayed in the live camera feed user interface 314 moves or deforms. For example, the feed shown in the live camera feed user interface 314 may show feed of body tissue that is flexible or deformable. If the electronic device 101 has applied an annotation to a portion of the surface of the physical object 310 and then later detects movement of that portion in the live camera feed user interface 314, it is desirable for that annotation to track that portion of the surface in the live camera feed user interface 314 (e.g., to maintain the integrity of the annotation as being on the portion of the object). In some cases, the camera 312 moves. In some examples, movement of the camera 312 is detected via an IMU sensor in communication with the camera 312. In some examples, the movement of the camera 312 is detected via image sensors of the electronic device 101. If the electronic device 101 has applied an annotation to a portion of the surface and then later detects movement of the camera 312, it is desirable for that annotation to track that portion of the object in the live camera feed user interface 314, to maintain the integrity of the annotation as being on the portion of the object. In some examples, the electronic device 101 performs an action with respect to a virtual annotation in response to detecting an event corresponding to relative movement between the camera 312 and the first portion of the physical object 310.

In FIG. 8J, the electronic device 101 displays first annotation 804a at the same location in the live camera feed user interface 314 as in FIG. 8C, and the annotated surface is at the same location in the live camera feed user interface 314 as in FIG. 8C. From FIG. 8J to FIG. 8K, the electronic device 101 detects an event corresponding to relative movement between the camera 312 and the first portion of the physical object 310. For example, the electronic device 101 may detect that the surface 437b of the physical object 310 has moved in the live camera feed user interface 314, resulting in movement of the surface that originally was at a first location in the live camera feed user interface 314. In response to detecting the event, the electronic device 101 moves the first annotation 804a in the live camera feed user interface 314 in accordance with the detected relative movement to maintain the spatial arrangement of the first annotation 804a and the surface originally requested to be annotated. If the movement of the first portion is movement to a location that is still inside of the field of view 313 of the camera 312, then the electronic device 101 may move display of the first annotation 804a in the live camera feed user interface 314 to another location inside in the live camera feed user interface 314 that corresponds to the new location of the first portion of the surface that is annotated, as shown in FIG. 8K. If the movement of the first portion is movement to a location that is outside of the field of view 313 of the camera 312, then the electronic device 101 may cease display of the first annotation in the live camera feed user interface 314 when it is moved outside of the field of view 313 of the camera 312, as shown in FIG. 8L.

In some examples, the surface (e.g., the first portion) to which the first annotation 804a corresponds has a first appearance (e.g., a first shape in the live camera feed user interface 314) and the first annotation 804a has a first annotation appearance (e.g., a first color, a first amount of transparency, a first brightness level, etc.) in the field of view of the camera 312. In some examples, the electronic device 101 detects that the surface has changed in appearance from the first appearance to a second appearance that is different from the first appearance. For example, a shape of the surface may have changed from a first shape to a second shape that is different from the first shape. In some examples, when the surface changes in shape, a level of confidence that the first annotation 804a applies to the surface decreases. In some examples, if the change in shape (e.g., the deformity) is a within a threshold change in shape (e.g., based on a comparison between the first shape and the second shape), the electronic device 101 may maintain display of the first annotation 804a having the first annotation appearance. In some examples, if the change in shape (e.g., the deformity) is beyond a threshold change in shape, the electronic device 101 may change display of the first annotation 804a to have a second annotation appearance (e.g., a second color, a second amount of transparency, a second brightness level) that is different from the first annotation appearance, or may cease display of the first annotation 804a altogether. In some examples, the second annotation appearance is a different color than the first annotation appearance. Additionally or alternatively, in some examples, the second annotation appearance has a higher amount of transparency than the first annotation appearance. Additionally or alternatively, in some examples, the second annotation appearance has a lower brightness level than the first annotation appearance. Additionally or alternatively, in some examples, the second annotation appearance is smaller in size than the first annotation appearance. Other differences between the second annotation appearance and the first annotation appearance are contemplated and are within the scope of the disclosure.

In some examples, the electronic device 101 displays a user interface 812 that indicates a level of confidence (e.g., a level of integrity) that the location of display of the first annotation 804a in the live camera feed user interface 314 corresponds to the location of the surface originally requested to be annotated. For example, in accordance with a determination that the confidence level is high, the user interface 812 indicates that the level of confidence is high; in accordance with a determination that the level of confidence is medium, the user interface 812 indicates that the level of confidence is medium (e.g., and not high); in accordance with a determination that the level of confidence is low, the user interface 812 indicates that the level of confidence is low (e.g., and not high or medium). Additionally or alternatively, in accordance with a determination that the level of confidence is medium or low, the electronic device 101 may display an indication requesting that the user 301 of the electronic device 101 annotate again. In some examples, the electronic device 101 reduces a visual prominence of the first annotation 804a in the live camera feed user interface 314 as a level of confidence is reduced.

In some examples, the electronic device 101 moves the first annotation 804a based on camera motion detection techniques. For example, if the electronic device 101 detects that the camera part 312a has moved three points rightward (e.g., rotated rightward without tangential movement), then the electronic device 101 may move the first annotation 804a in the live camera feed user interface 314 three points to the left. In some examples, the electronic device uses SLAM map localization to detect camera motion.

In some examples, the electronic device moves the first annotation based on object recognition detection techniques. For example, the electronic device 101 may use an object recognition system that identifies a surface in the live camera feed user interface 314, such as surface 437b, and may detect that the surface 437b has moved in the field of view 313 of the camera 312.

In some cases, users of electronic devices may desire to collaborate with each other. For example, as described with reference to FIGS. 3F and 3G, user 301 may desire to collaborate with “Dr. 1”. In some cases, a first user is in the physical presence of the physical object 310 and a second user is not in the physical presence of the physical object 310 (e.g., the second user is remote from the location of the first user and the physical object 310). It may be desirable for the second user to see and/or provide input regarding one or more operations to be performed on the physical object 310. In some examples, the electronic device 101 provides for recording the three-dimensional environment presented by the electronic device 101 to the user 301. For example, the electronic device 101 may record the three-dimensional environment of the user 301 that is presented at the electronic device 101, including that of live camera feed user interface 314, annotations made by the user 301, and of the external view of the physical object 310. For example, the electronic device 101 may record the field of view of the electronic device 101 that is visible via display 120 in FIG. 8L. In some examples, while recording the field of view of the electronic device 101 that is visible via display 120, the electronic device 101 displays an indication 814 that the electronic device 101 is recording the field of view, as shown in FIG. 8L. In some examples, the electronic device 101 transmits (e.g., uploads to a data storage system) the recording to a location that is accessible by the second user of a second electronic device, so that the second user can view the recording. In some examples, when the recording is in playback, it is two-dimensional. In some examples, when the recording is in playback, it is three-dimensional.

In some cases, different users of electronic devices may operate on the physical object 310 at different times. For example, a first user of the electronic device 101 may operate on the physical object 310 at a first time (e.g., at a first hour of a first day), and a second user of the electronic device 101 may operation on the physical object 310 at a second time that is after the first time (e.g., at a fifth hour of the first day, or at another hour or day that is after the first hour of the first day). Continuing with this example, it may be desirable for the second user to view and/or access virtual annotations made by the first user. In some examples, the electronic device 101 provides for conserving annotations made between different users of electronic devices so that new users can view annotations made by previous users. For example, user 301 may be a first user and may have created the first annotation 804a while operating on physical object 310. After user 301 is finished operating on physical object 310, a second user may operate on physical object 310 and the second electronic device of the second user may display a live camera feed user interface 314. If while operating on the physical object 310, the second electronic device detects that the location of the surface of physical object 310 originally requested to be annotated by the first user is in the live camera feed user interface 314, the second electronic device may display the first annotation 804a that was made by the first user at that location.

In some cases, while user 301 (e.g., a first user of a first electronic device) is operating on the physical object 310, user 301 may desire input from a second user of a second electronic device who is not in the physical presence of the first user 301 (or of the physical object 310). In some examples, as shown in FIG. 3G, the electronic device 101 may display the second user (e.g., representation 326b in FIG. 3G), and may cause the second electronic device of the second user (e.g., the computer system associated with “Dr. 1”) to display the live camera feed user interface 314.

In some examples, the electronic device 101 transmits to the second electronic device an environment including virtual representation of the physical object 310, optionally in addition to the transmission of the live camera feed. In some examples, the environment is two-dimensional. In some examples, the environment is three-dimensional. For example, the electronic device 101 optionally transmits a three-dimensional model of the physical object 310 including its internal surfaces, and the second electronic device may detect input from the second user requesting an annotation on a respective surface of the three-dimensional model of the physical object. In response, the second electronic device may annotate the respective portion. In some examples, the second electronic device transmits the three-dimensional model of the portion of the physical object, including the annotations that may have been made by the second user on the model to the electronic device 101 (or to another electronic device) so that another user can view the annotated model.

In some examples, the second electronic device displays a live camera feed user interface (e.g., live camera feed user interface 314) and permits the second user to annotate in the live camera feed user interface. For example, the live camera feed user interface 314 that is displayed by the electronic device 101 may also be displayed elsewhere by a second electronic device that is remote from the physical environment of the three-dimensional environment 300, and both user interfaces may be updated in response to annotation inputs made by the users (e.g., either or both users) of the electronic devices. For instance, in some examples, the electronic device 101 responds to annotations made by the second user of the second electronic device by updating display of live camera feed user interface 314 (that is displayed by the electronic device 101) to include the annotations made by the second user of the second electronic device (e.g., while a live camera feed user interface of the physical object 310 is being displayed by the second electronic device remote from the physical environment of the three-dimensional environment 300). For example, the input requesting the first annotation 804a could have alternatively been detected by the second electronic device as input from the second user (e.g., who is remote from the physical object 310), and in response the electronic device 101 may display the first annotation 804a in live camera feed user interface 314 as well. In some examples, the electronic device 101 visually differentiates between annotations made by different users so that the different users can determine who made the annotation. In some examples, the electronic device 101 does not visually differentiate between annotations made by different users.

FIG. 8M is a flow diagram illustrating a method 850 for displaying an annotation in a user interface that includes a render of camera feed showing a portion of an object, and for moving the annotation in response to detecting an event corresponding to relative movement between the camera and the portion of the object according to some examples of the disclosure. It is understood that method 850 is an example and that more, fewer, or different operations can be performed in the same or in a different order. Additionally, the operations in method 850 described below are, optionally, implemented by running one or more functional modules in an information processing apparatus such as general-purpose processors (e.g., as described with respect to FIG. 2) or application specific chips, and/or by other components of FIG. 2.

Therefore, according to the above, some examples of the disclosure are directed to a method (e.g., method 850 of FIG. 8M) including at an electronic device in communication with one or more displays and one or more input devices, including a camera, presenting (852), via the one or more displays, a view of a physical environment of the first electronic device from a viewpoint of the first electronic device in the physical environment, the view of the physical environment including an external view of a physical object, while presenting the view of the physical environment, displaying (854), via the one or more displays, a first user interface Including a video feed from the camera, wherein a location of the camera corresponds to a location of the physical object (e.g., the camera is inside the physical object), while displaying the first user interface including the video feed from the camera, detecting (856) a first input to create a virtual annotation associated with a first portion of the physical object that is in the video feed from the camera, in response to detecting the first input, creating (858) the virtual annotation associated with the first portion of the physical object that is in the video feed from the camera, including updating display, via the one or more displays, of the first user interface to include the virtual annotation associated with the first portion of the physical object that is in the video feed from the camera, while displaying the updated first user interface, detecting (860) an event corresponding to relative movement between the camera and the first portion of the physical object that is in the video feed from the camera, and in response to detecting the event, moving (862) the virtual annotation associated with the first portion of the physical object in accordance with the relative movement between the camera and the first portion of the physical object that is in the video feed from the camera.

Additionally or alternatively, in some examples, the first portion is a point on a surface of the physical object that is in the video feed from the camera when the first input is detected, and updating display of the first user interface to include the virtual annotation includes displaying the virtual annotation on the point.

Additionally or alternatively, in some examples, the first portion is an area defined according to a plurality of points on one or more surfaces of the physical object that are in the video feed from the camera when the first input is detected, and updating display of the first user interface to include the virtual annotation includes displaying the virtual annotation overlaid on the area.

Additionally or alternatively, in some examples, the first portion corresponds to two points on one or more surfaces in the physical object that are in the video feed from the camera when the first input is detected, the first input includes a request to determine a distance between the two points, and updating display of the first user interface to include the virtual annotation includes displaying an indication of the distance between the two points in the first user interface.

Additionally or alternatively, in some examples, the one or more input devices includes an audio input device, and wherein the first input is detected via the audio input device.

Additionally or alternatively, in some examples, the event includes movement of the camera in the physical environment.

Additionally or alternatively, in some examples, the event includes movement of the first portion in the physical environment and/or a change in a shape of the first portion in the physical environment.

Additionally or alternatively, in some examples, the event includes movement of the camera in the physical environment and movement of the first portion in the physical environment.

Additionally or alternatively, in some examples, the first electronic device is in communication with a second electronic device, and the method 850 comprises while presenting the view of the physical environment of the first electronic device and while displaying the first user interface or the updated first user interface, causing display, at the second electronic device, of a three-dimensional representation of the view of the physical environment of the first electronic device, including a representation of the first user interface or the updated first user interface. Additionally or alternatively, in some examples, the first input is detected at the second electronic device via one or more second input devices that are in communication with the second electronic device before being detected at the first electronic device, and detecting the first input at the first electronic device includes detecting that the first input was detected at the second electronic device. Additionally or alternatively, in some examples, the first input is detected at the first electronic device via the one or more input devices before being detected at the second electronic device, and detecting the first input at the second electronic device includes detecting that the first input was detected at the first electronic device.

Additionally or alternatively, in some examples, the first electronic device is located in the same physical environment as the physical object and the second electronic device is remote from the physical environment.

Additionally or alternatively, in some examples, the method 850 includes detecting a second input to create a virtual annotation associated with a second portion of the physical object, different from the first portion of the physical object and in response to detecting the second input, creating the virtual annotation associated with the second portion of the physical object, including updating display, via the one or more displays, of the first user interface to include the virtual annotation associated with the second portion of the physical object.

Additionally or alternatively, in some examples, the method 850 includes saving the virtual annotation associated with the first portion.

Additionally or alternatively, in some examples, the video feed from the camera is stereo video feed.

Additionally or alternatively, in some examples, the camera is laparoscopic camera and the physical object is a body of a patient.

Additionally or alternatively, in some examples, the first electronic device includes a head-mounted display system.

Attention is now directed towards examples of an electronic device displaying models of objects, detecting and responding to input for rotating the models of objects, and detecting and responding to input for displaying different amounts of the models of the objects in accordance with some examples.

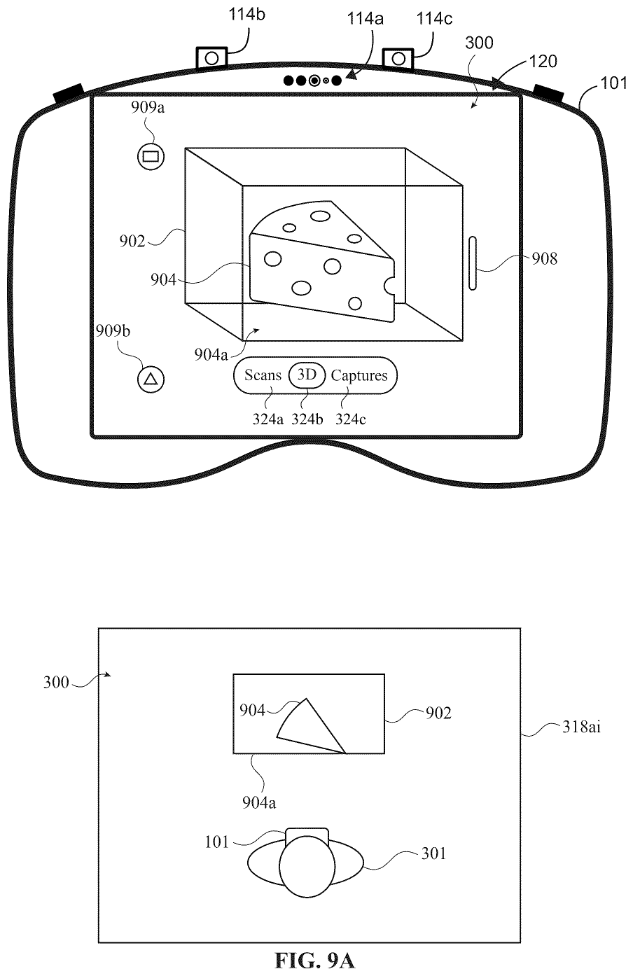

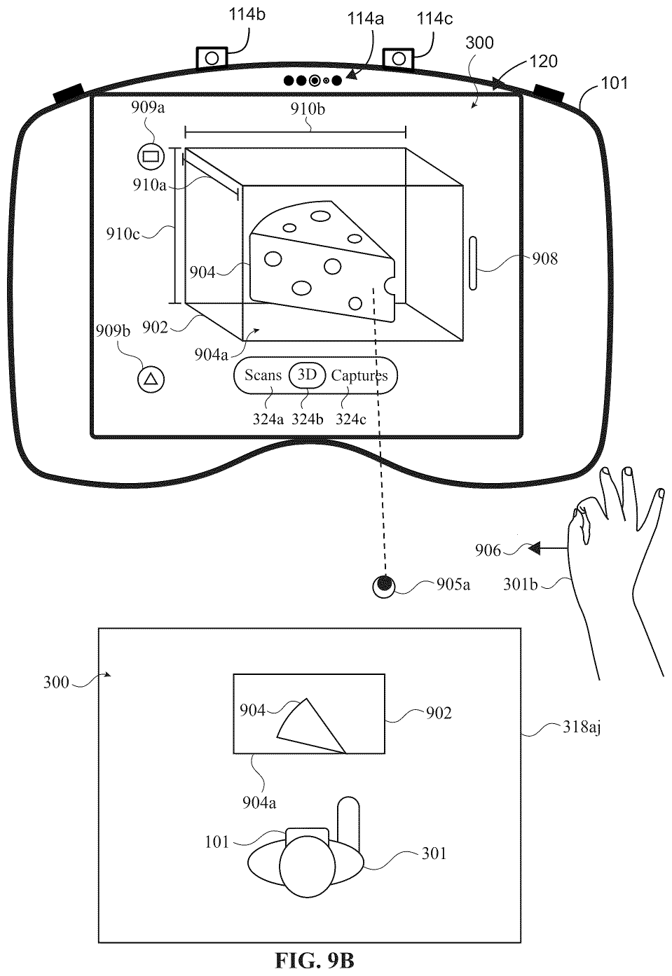

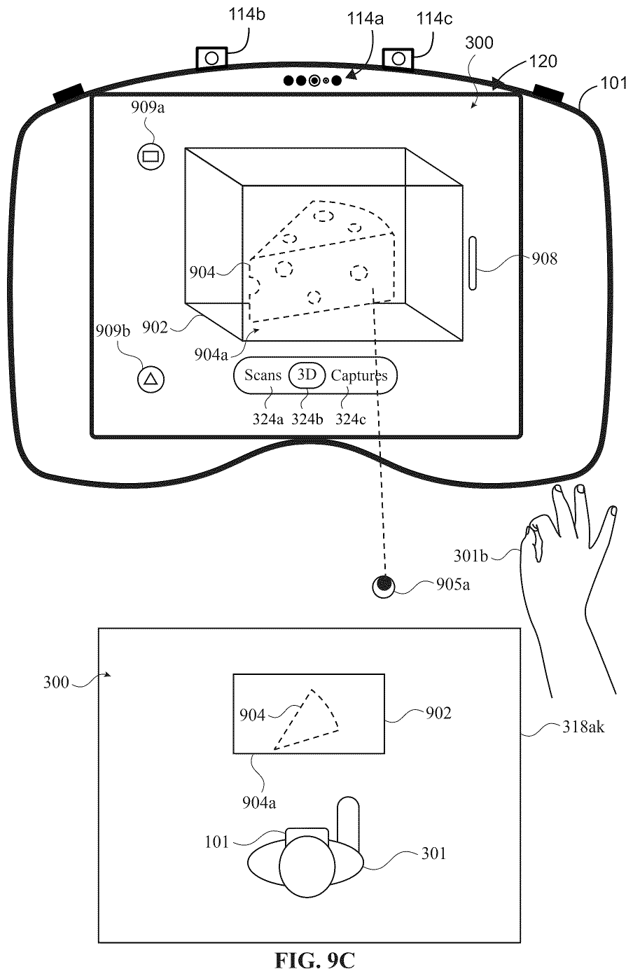

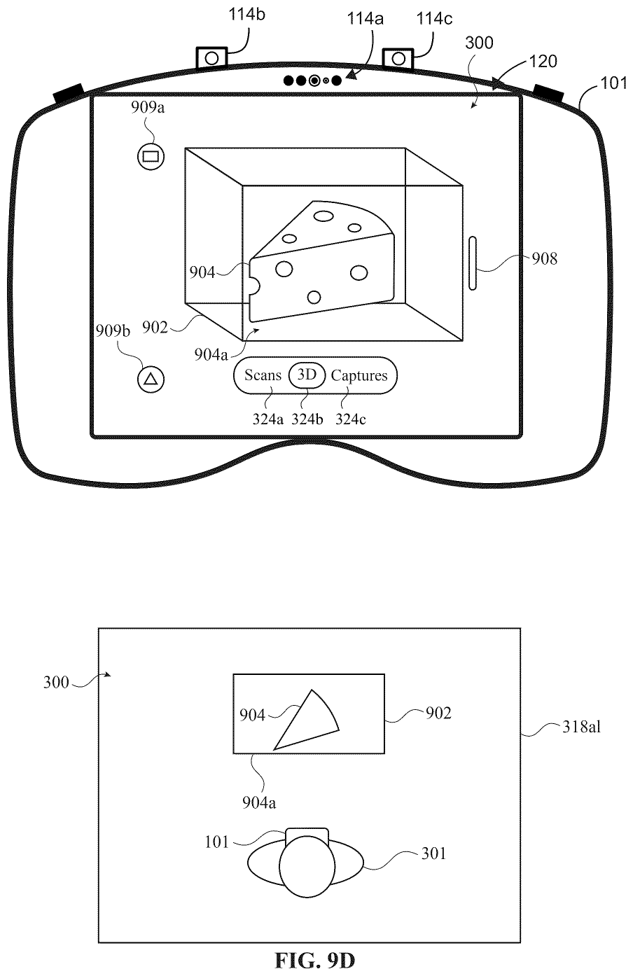

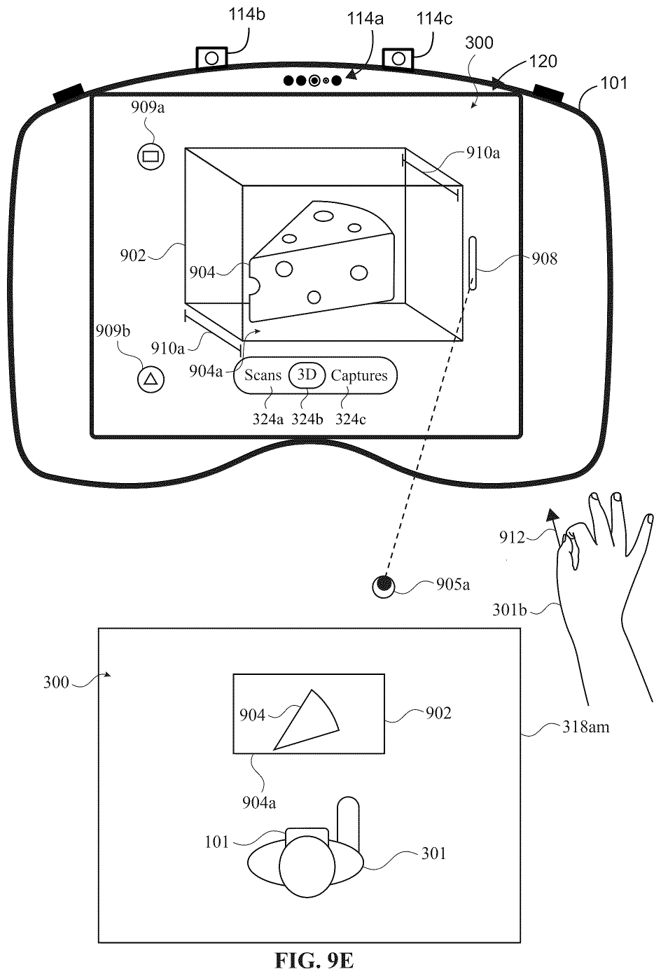

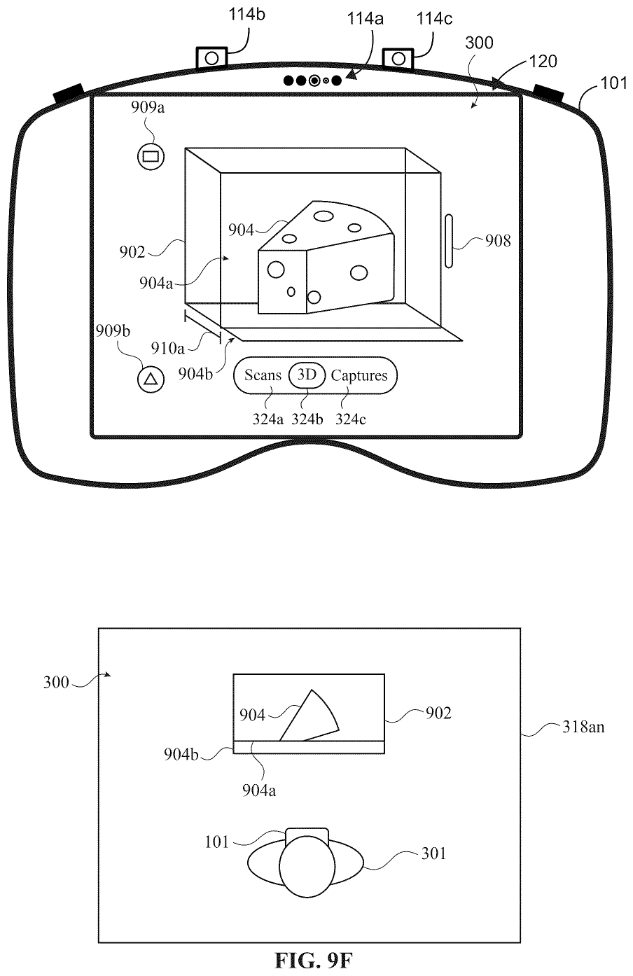

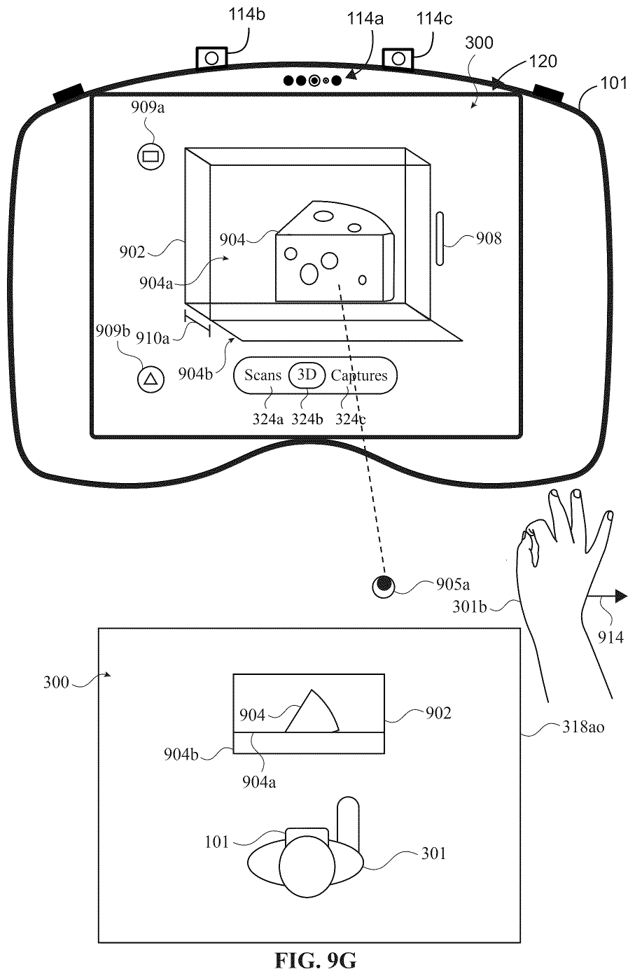

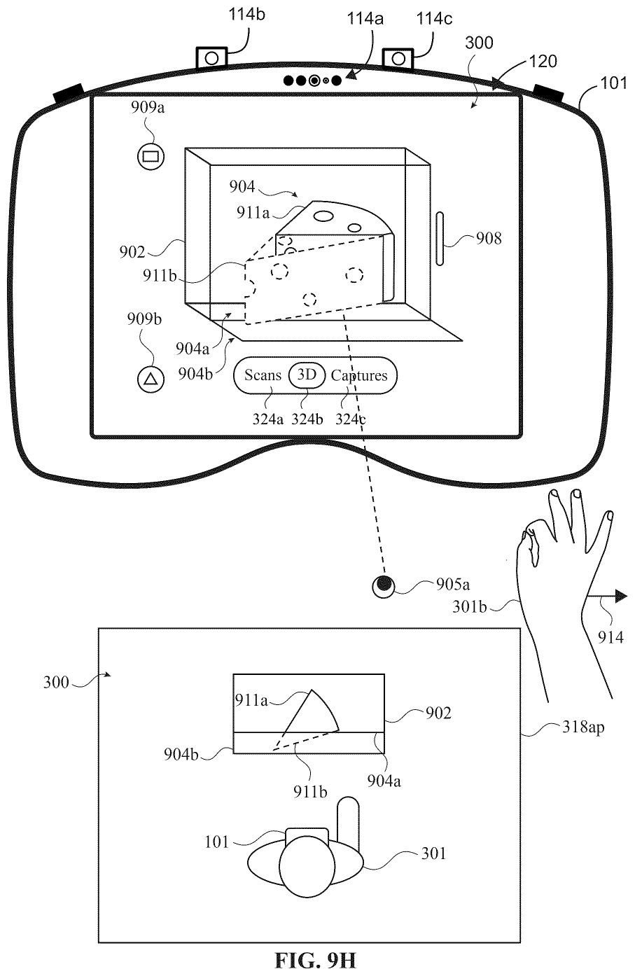

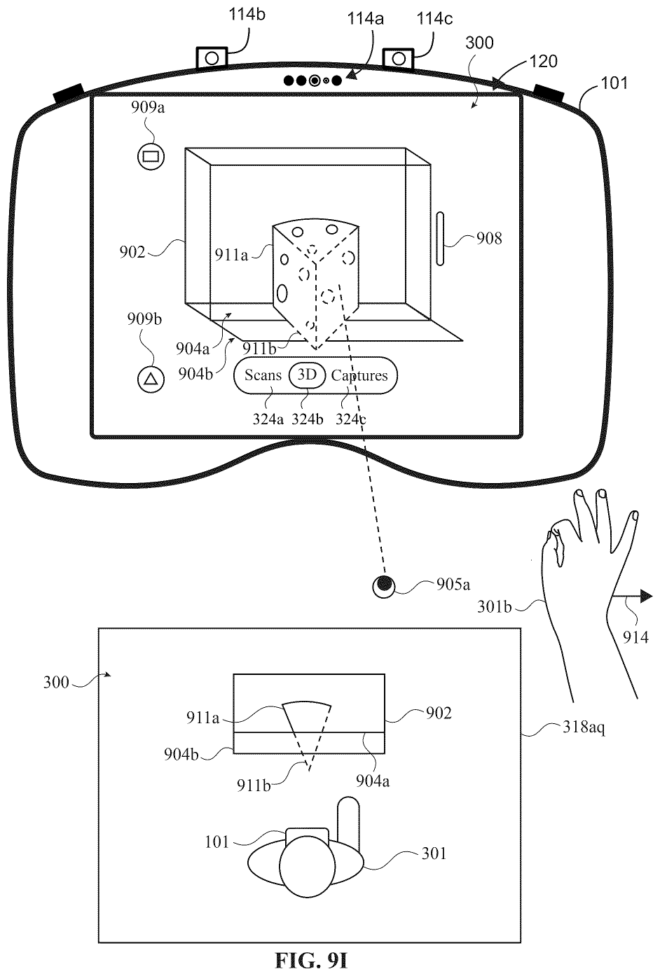

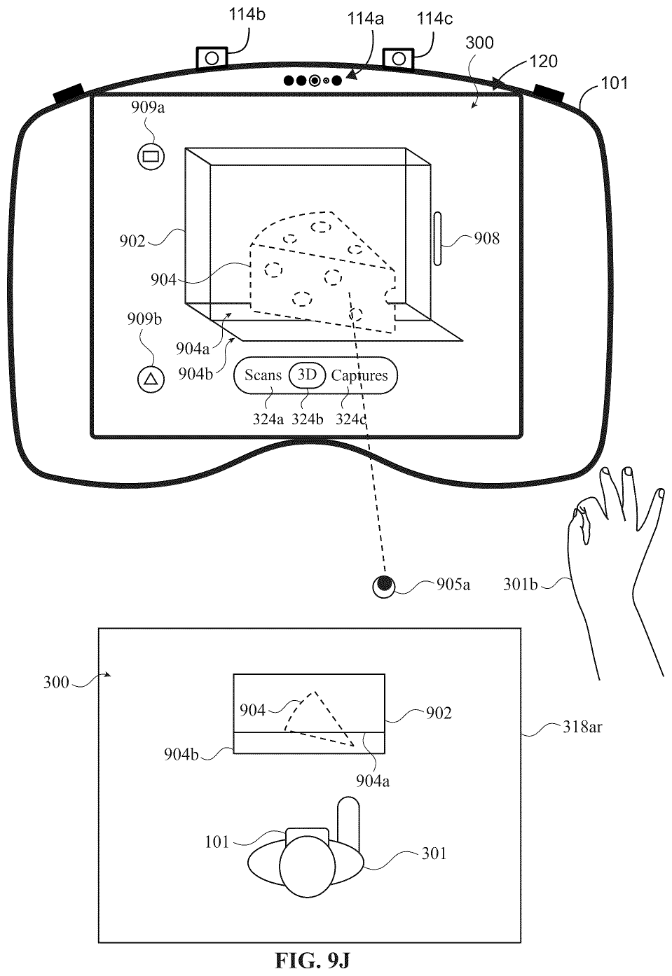

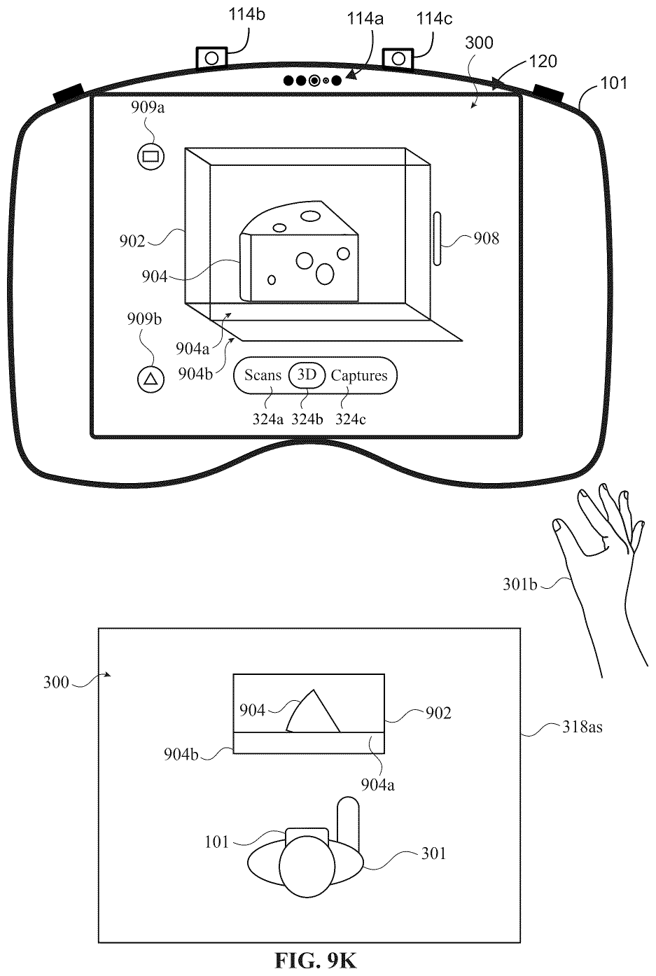

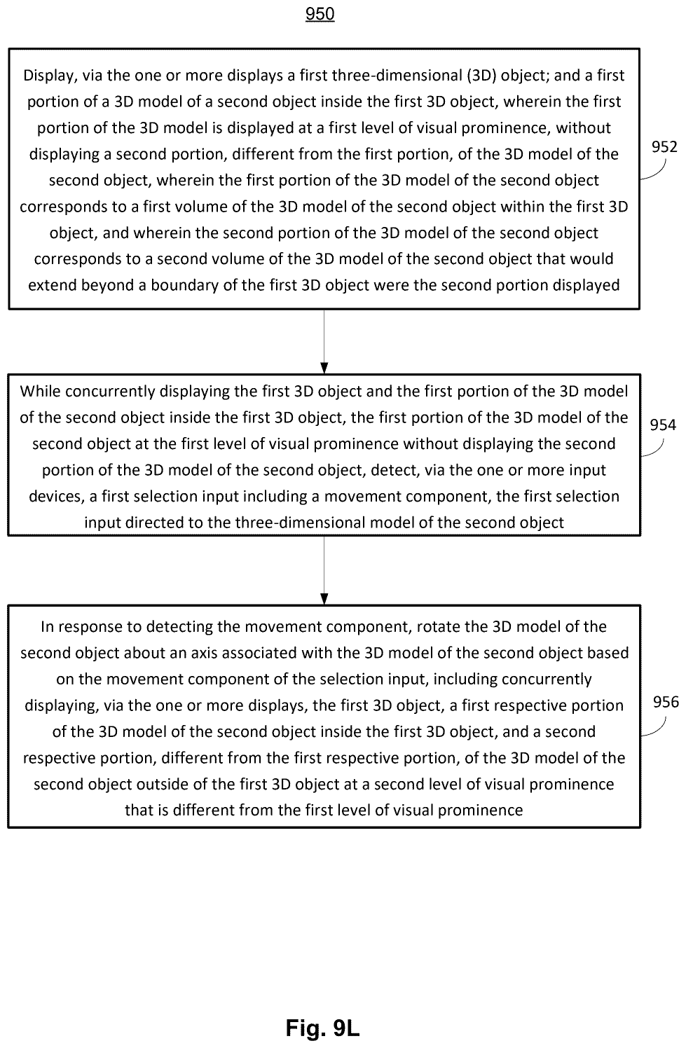

In some cases, it is desirable for users to view models of objects (e.g., models of physical objects). For example, a user who will be operating on physical object 310 may desire to see a three-dimensional model of the physical object 310 (or of a portion of an object inside of physical object 310) to assist the user in preparing for the operation that is to be performed on the physical object 310 and/or to assist the user in the operation that the user is currently performing on the physical object 310. In some examples, an electronic device displays a model of an object concurrently with display of the live camera feed user interface 314, such as shown in FIG. 3G with display of 3D object 322b and box 322a. In some examples, an electronic device displays the model of the object without display of the live camera feed user interface 314, such as shown in FIG. 9A. In some examples, the electronic device detects and responds to input for rotating the model by rotating the model. In some examples, the electronic device detects and responds to input for viewing the model from different depth positions within the model.

FIGS. 9A-9K illustrates examples of an electronic device displaying a 3D model of an object, and detecting and responding to input for viewing the model from different depth positions within the model in accordance with some examples.

For the purpose of illustration, FIGS. 9A-9K include respective top-down views 318ai-318as of the three-dimensional environment 300 that indicate the positions of various objects (e.g., real and/or virtual objects) in the three-dimensional environment 300 in a horizontal dimension and a depth dimension. The top-down view of the three-dimensional environment 300 further includes an indication of the viewpoint of the user 301 of the electronic device 101. For example, in FIG. 9A, the electronic device 101 displays the view of the three-dimensional environment 300 visible through the display 120 from the viewpoint of the user 301 illustrated in the top-down view 318ai of the three-dimensional environment 300.

FIG. 9A illustrates the electronic device 101 concurrently displaying a first 3D object 902 (e.g., box 322a of FIG. 3E) and a second 3D object 904 (e.g., 3D object 322b of FIG. 3E) inside the first 3D object 902. The second 3D object is a 3D model of an object. In FIG. 9A, the 3D model of the second object is a 3D model of a slice of Swiss cheese, which is representative and nonlimiting. In FIG. 9A, a location of the side 904a of the first 3D object 902 corresponds to a location of depth position of the second 3D object 904 that is a minimal or zero depth relative to the second 3D object 904. For example, in FIG. 9A, the total volume of the second 3D object 904 is inside the first 3D object 902. That is, in FIG. 9A, no portion of the second 3D object 904 would be displayed outside of the side of the first 3D object 902 because the first 3D object 902 fully encloses the second 3D object 904. In FIG. 9A, a level of visual prominence of the second 3D object 904 is a first level of visual prominence (e.g., a first level of contrast, brightness, saturation, opacity, and/or visual emphasis). In FIG. 9A, a volume of the first 3D object 902 is greater than a volume of the second 3D object 904. In FIG. 9A, the first 3D object 902 has no fill. In some examples, the first 3D object 902 has a transparent or semi-transparent fill. In FIG. 9A, the electronic device 101 also displays user interface elements 324a through 324c, which are as described with reference to FIG. 3C. Further, in FIG. 9A, the electronic device 101 also displays a first user interface element 909a and a second user interface element 909b. In some examples, the first user interface element 909a is selectable to perform one or more of the operations described with reference to selection of any of the user interface elements 316a-316d. In some examples, the second user interface element 909b is selectable to present options to the user 304 for changing one or more characteristics of the three-dimensional environment 300 that is displayed via display 120.

In FIG. 9B, while concurrently displaying the first 3D object 902 and the second 3D object 904 inside the first 3D object 902, as in FIG. 9A, the electronic device 101 detects a first selection input. In FIG. 9B, the first selection input includes the hand 301b of the user 301 performing an air pinch gesture (e.g., index finger of the user 301 touching the thumb of the user 301 and maintaining contact) while a gaze 905a of the user 301 is directed to the second 3D object 904. In FIG. 9B, the first selection input includes a movement component that includes movement of the hand 301b of the user 301 while it is in the pinch pose (e.g., while contact of the index finger and the thumb is maintained), as illustrated with the arrow 906. For example, the movement component may include lateral movement of the hand 301b of the user 301 relative to the torso of the user 301. In some examples, in response to detecting the movement component of the first selection input of FIG. 9B, the electronic device 101 performs a rotation animation, such as shown in FIGS. 9C through 9E.

From FIG. 9B to FIG. 9C, the electronic device 101 rotates the second 3D object 904 about an axis associated with the second 3D object 904 in accordance with the movement component of the first selection input in response to detecting the movement component of the first selection input of FIG. 9B. For example, the second 3D object 904 has been rotated by 90 degrees clockwise, as shown from top-down view in FIG. 9B to the top-down view in FIG. 9C. In some examples, the electronic device 101 rotates the second 3D object 904 in a direction that is based on a direction that the hand 301b moves while the first selection input is being detected.

In addition, the electronic device 101 has changed a visual prominence of the second 3D object 904 in response to detecting the movement component of the first selection input of FIG. 9B. For example, in FIG. 9A, the electronic device 101 displays the second 3D object 904 at the first level of visual prominence described above, and in FIG. 9C, the electronic device 101 displays the second 3D object 904 at a second level of prominence (e.g., a second level of contrast, brightness, saturation, opacity, and/or visual emphasis) that is different from the first level of visual prominence. In the illustrated example of FIG. 9C, the second level of visual prominence is less than the first level of visual prominence. In some examples, the second level of visual prominence is greater than the first level of visual prominence. In some examples, the electronic device 101 may change the visual prominence of the second 3D object 904 from the first level to the second level while the second 3D object 904 is being rotated and/or when the movement component (e.g., when a part of the movement component) is initially detected. For example, from FIG. 9B to FIG. 9C, the second 3D object 904 is rotated by 90 degrees as described above, and at any intermediate orientation transgressed by the second 3D object 904 while the first selection input is being received, the electronic device 101 is displaying the second 3D object 904 at the second level of visual prominence, and/or is displaying at least a portion of the second 3D object 904 at the second level of visual prominence and increases the amount of the second 3D object 904 that is displayed at the second level of visual prominence until the displayed second 3D object 904 is fully at the second level of visual prominence. Note that, in some examples, the electronic device 101 displays a user interface element indicative of an orientation of the second 3D object 904 relative to the first 3D object 902. For example, in response to detecting the movement component, the electronic device 101 may display the user interface element. In some examples, the user interface element is a slider or a pie with a fill that is based on an orientation of the second 3D object relative to the first 3D object 902. For example, were the second 3D object 904 to have a first orientation, the pie would have a first amount of fill, and were the second 3D object 904 to have a second orientation that is different from the first orientation, the pie would have a second amount of fill that is different from the first amount of fill. As such, the amount of fill may change in response to rotation of the second 3D object 904.

From FIG. 9C to FIG. 9D, the electronic device 101 detects that the first selection input of FIG. 9B has concluded while the second 3D object 904 has been rotated as shown in FIG. 9C. For example, the electronic device 101 detects that the hand 301b of the user 301 that was in the air pinch pose in FIG. 9B is no longer in the air pinch pose and corresponds that detection to conclusion of the first selection input. In response to detecting conclusion of the first selection input, the electronic device 101 may display the second 3D object 904, as rotated in accordance with the movement component, with the first level of visual prominence, as shown in FIG. 9D. As such, in some examples, in response to detecting conclusion of the first selection input, the electronic device 101 changes the visual prominence of the second 3D object 904 from the second level to the first level, as shown from FIG. 9C to FIG. 9D.

In some examples, the electronic device 101 displays a part of the second 3D object 904 that is beyond a depth position within the second 3D object 904, without displaying a part of the second 3D object 904 that is not beyond a depth position within the second 3D object 904. In some examples, a location of the side 904a of the first 3D object 902 indicates the depth within the second 3D object 904 at which the second 3D object 904 is being displayed by the electronic device 101. In FIG. 9A, the side 904a is at a first location that corresponds to a minimum or zero depth within the second 3D object 904 (e.g., based on the orientation of the second 3D object 904 in FIG. 9A). Were the second 3D object 904 oriented differently in first 3D object 902 in FIG. 9A, the depth position within the second 3D object 904 may be different (e.g., nonzero).

In some examples, the electronic device 101 detects and responds to inputs for viewing the second 3D object 904 from different depths within the second 3D object 904. In some examples, the electronic device 101 displays user interface element 908, which is selectable to change a depth within the second 3D object 904 at which the second 3D object 904 is displayed. As such, in some examples, the user interface element 908 is selectable to change a depth within the second 3D object 904 at which the second 3D object 904 is being displayed by the electronic device 101. Additionally, the user interface element 908 is selectable to change a location at which side 904a of the first 3D object 902 is displayed, as described below.

In some examples, the electronic device 101 displays the first 3D object 902 to provide an indication of a sense of depth (and/or of other dimensions) of the second 3D object 904. The user interface element 908 is selectable to set a boundary of the first 3D object 902 (e.g., to set a location of the side 904a of the first 3D object 902). The first 3D object 902 in FIG. 9B has a length 910a, width 910b, and a height 910c, and the user interface element 908 is selectable to set the length 910a of the first 3D object 902, while the width 910b and the height 910c may not be changed. The depth position from which the second 3D object 904 is being displayed is based on a location of the side 904a of the first 3D object. Were the length 910a a first length (e.g., the location of the side 904a a first location), the electronic device 101 would display the second 3D object 904 from a first depth within the second 3D object 904, and were the length 910a set to a second length (e.g., the location of the side 904a a second location), different from the first length, the electronic device 101 would display the second 3D object 904 from a second depth that is different from the first depth. The greater the length 910a, the smaller the depth position from which the second 3D object 904 is being displayed. The smaller the length 910a, the greater the depth position from which the second 3D object 904 is being displayed. Additionally, in the illustrated examples, were the length 910a a first length, the first 3D object 902 would have a first volume, and were the length 910a set to a second length, different from the first length, the first 3D object 902 would have a second volume different from the first volume. For example, were the length 910a a first length that is greater than a second length, the first 3D object 902 would have a first volume that is greater than a second volume, and were the length 910a the second length, the first 3D object 902 would have the second volume that is less than the first volume.

As described above, in some examples, the portion of the second 3D object 904 that is displayed by the electronic device 101 is the portion of the second 3D object 904 that has a position that is beyond (e.g., at or greater than) the depth position set by the location of the side 904a of the first 3D object 902 (e.g., based on the orientation of the second 3D object 904 inside the first 3D object 902). For example, in FIG. 9A, the electronic device 101 is displaying the portion of the second 3D object that is at or greater than the depth position given by the location of the side 904a of the first 3D object 902. In other words, the depth component of the coordinates of the second 3D object 904 is at or beyond the corresponding depth component of the location of the side 904a of the first 3D object 902 in FIG. 9A. As such, the location of the side 904a of the first 3D object 902 may indicate the depth within the second 3D object 904 at which the second 3D object 904 is being displayed by the electronic device 101. Such features are also described with reference to FIGS. 9E and 9F.

FIGS. 9E and 9F illustrate an example of the electronic device 101 detecting and responding to input for changing a depth within the second 3D object 904 at which the second 3D object 904 is being displayed by the electronic device 101.

In FIG. 9E, while concurrently displaying the first 3D object 902 and the second 3D object 904 inside the first 3D object 902, as in FIG. 9D, the electronic device 101 detects a second selection input. In FIG. 9E, the second selection input includes the hand 301b of the user 301 performing an air pinch gesture (e.g., index finger of the user 301 touching the thumb of the user 301 and maintaining contact) while a gaze 905b of the user 301 is directed to the user interface element 908. In FIG. 9E, the second selection input includes a movement component including movement of the hand 301b of the user 301 while it is in the pinch pose, as illustrated with the arrow 912. In some examples, the movement component includes movement towards the location of the user interface element 908, as illustrated with the arrow 912. In some examples, in response to detecting the movement component of the second selection input, the electronic device 101 changes the depth within the second 3D object 904 at which the second 3D object 904 is being displayed, such as shown from FIG. 9E to FIG. 9F.

From FIG. 9E to FIG. 9F, the electronic device 101 has reduced the magnitude of the length 910a of the first 3D object 902 (e.g., without changing a magnitude and location of the width and height of the first 3D object 902), thus changing a location of the side 904a of the first 3D object 902. Additionally, since the length 910a is reduced from FIG. 9E to FIG. 9F while the magnitude and location of the width and height are constant, the volume of the first 3D object 902 in FIG. 9F is less than the volume of the first 3D object 902 in FIG. 9E. Note that, though length 910a of the first 3D object 902 has been reduced from FIG. 9E to 9F, the electronic device 101 maintains display of side 904b of first 3D object 902 having the same length as in FIG. 9D to provide the user with a depth indication. As such, side 904b of the first 3D object 902 extends beyond the intersection of side 904b with side 904a in FIG. 9F.

Further, from FIG. 9E to FIG. 9F, the electronic device 101 has increased the depth within the second 3D object 904 at which the second 3D object 904 is being displayed. For example, in FIG. 9E, the depth within the second 3D object 904 at which the second 3D object 904 is being displayed may be a minimum or zero depth, and in FIG. 9F, the depth the second 3D object 904 at which the second 3D object 904 is being displayed is greater than in FIG. 9E. As such, in FIG. 9F, the portion of the second 3D object 904 that is displayed is the portion that is beyond the depth position that corresponds to the location of the side 904a of the first 3D object 902 in FIG. 9F.

Note that a direction of change of magnitude of the length 910a and a direction of change of the depth within the second 3D object at which the second 3D object 904 is being displayed may be based on a direction associated with the movement component. For example, were the movement component associated with a first direction, such as toward the user interface element 908, the electronic device 101 would cause the directions of the changes to be as illustrated from FIG. 9E to FIG. 9F. Continuing with this example, were the movement component associated with a second direction, such as away from the user interface element 908, the electronic device 101 would cause the directions of the changes to be the opposite of the illustrated directions of changes from FIG. 9E to FIG. 9F. For example, were the electronic device 101 to detect a selection input directed to the user interface element 908 including a movement component that is in the opposite direction of the arrow 912, the electronic device 101 would cause the directions of the changes to be the opposite of the illustrated directions of changes from FIG. 9E to FIG. 9F.

In some examples, FIGS. 9E-9G illustrate an example of the electronic device 101 detecting different amounts of movement components of the second selection input. For example, were the movement component of the second selection input of FIG. 9E a first amount, the electronic device 101 would respond by changing the depth within the second 3D object at which the second 3D object 904 is being displayed to a first depth, as shown from FIG. 9E to FIG. 9F. Continuing with this example, were the movement component of the second selection input of FIG. 9E a second amount that is greater than the first amount, the electronic device 101 would respond by changing the depth within the second 3D object at which the second 3D object 904 is being displayed to a second depth that is greater than the first depth, as shown from FIG. 9E to FIG. 9G. Note that the electronic device 101 may visually show progression of the change of depth. For example, were the movement component of the second selection input of FIG. 9E the second amount described above, the electronic device 101 would display the depth changing, including changing from the first depth described above to the second depth described above. As such, the electronic device 101 may display the second 3D object 904 from intermediate depths until a final depth position associated with the movement component of the selection input is reached.

FIGS. 9G-9I illustrate an example of the electronic device 101 detecting and responding to a third selection input that includes a movement component, in accordance with some examples.

In FIG. 9G, while concurrently displaying the first 3D object 902 and the second 3D object 904 inside the first 3D object 902, the electronic device 101 detects a third selection input (e.g., different from the second selection input and/or after the second selection input is complete). In FIG. 9G, the third selection input includes the hand 301b of the user 301 performing an air pinch gesture (e.g., index finger of the user 301 touching the thumb of the user 301 and maintaining contact) while a gaze 905c of the user 301 is directed to the second 3D object 904. In FIG. 9G, the third selection input includes a movement component that includes movement of the hand 301b of the user 301 while it is in the pinch pose, as illustrated with the arrow 914. In some examples, the movement component includes lateral movement of the hand 301b of the user 301. Note that, in some examples, the electronic device 101 detects the hand 301b of the user 301 performing the air pinch gesture while the gaze of the user 301 is directed to the second 3D object 904 before it detects the movement component of the third selection input. In some examples, in response to detecting the movement component of the third selection input, the electronic device 101 performs a rotation animation, such as shown from FIG. 9H to 9I.

From FIG. 9G to 9H, the electronic device 101 has rotated the second 3D object 904 by a first amount, and has started displaying a portion of the second 3D object 904 that extends outside of the side 904a of the first 3D object 902 based on the orientation of the second 3D object 904 in FIG. 9H. In particular, in FIG. 9H, the displayed second 3D object 904 includes a first portion 911a, which corresponds to a first volume of the second 3D object 904 that is within the first 3D object 902, and includes a second portion 911b, which corresponds to a second volume of the second 3D object 904 that is in front of the side 904a of the first 3D object 902. Note that the second portion 911b was not displayed in FIG. 9G. Further, the second portion 911b is displayed at the second level of visual prominence and while the first portion 911a is displayed at the first level of visual prominence. In some examples, as the second 3D object 904 is rotated, the electronic device 101 reduces the amount of the second 3D object 904 that is displayed at the first level of visual prominence and increases the amount of the second 3D object 904 that is displayed at the second level of visual prominence, such as shown from FIG. 9H to FIG. 9I.

From FIG. 9H to FIG. 9I, the electronic device 101 is rotating the second 3D object 904 in response to the movement component, and is reducing the amount of the second 3D object 904 that is displayed at the first level of visual prominence and increasing the amount of the second 3D object 904 that is displayed at the second level of visual prominence. For example, part of the first portion 911a of the second 3D object 904 that was displayed at the first level of visual prominence in FIG. 9H is being displayed at the second level of visual prominence in FIG. 9I. In some examples, in response to the movement component, were the portion of the second 3D object 904 that was displayed when the movement component was detected moved to a location that is no longer inside the first 3D object 902, the electronic device 101 would display the second 3D object 904 at the second level of visual prominence, without displaying a portion of the second 3D object 904 at the first level of visual prominence, such as shown in FIG. 9J.

From FIG. 9I to FIG. 9J, the electronic device 101 has further rotated the second 3D object 904. In FIG. 9J, the electronic device 101 concurrently displays the first 3D object 902 and the second 3D object 904 including portions of the second 3D object 904 inside the first 3D object 902 and portions the second 3D object 904 outside of the side (e.g., the side 904a) of the first 3D object 902. In FIG. 9J, the electronic device 101 is displaying the second 3D object 904 at the second level of visual prominence without displaying a portion the second 3D object 904 at the first level of visual prominence.

In FIG. 9K, the electronic device 101 detects that the third selection input has concluded while the second 3D object 904 has the same orientation as in FIG. 9J. For example, the electronic device 101 may detect that the third selection input is concluded when the hand 301b of the user 301 is no longer in the air pinch gesture, such as shown in FIG. 9K. In response to detecting conclusion of the third selection input, the electronic device 101 may cease displaying the portion of the second 3D object 904 that is outside of the side 904a of the first 3D object 902 when conclusion of the third selection input was detected, and may maintain display of the remaining portion of the second 3D object 904 that is inside of the first 3D object 902 when conclusion of the third selection input was detected, as shown in FIG. 9K. Additionally, in response to detecting conclusion of the third selection input, the electronic device 101 changes the visual prominence of the second 3D object 904 that was displayed inside the first 3D object 902 when conclusion of the third selection input was detected from the second level of visual prominence to the first level of visual prominence, as from FIG. 9J to FIG. 9K.

FIG. 9L is a flow diagram illustrating a method 950 for displaying an annotation in a user interface that includes a render of camera feed showing a portion of an object, and for moving the annotation in response to detecting an event corresponding to relative movement between the camera and the portion of the object according to some examples of the disclosure. It is understood that method 850 is an example and that more, fewer, or different operations can be performed in the same or in a different order. Additionally, the operations in method 850 described below are, optionally, implemented by running one or more functional modules in an information processing apparatus such as general-purpose processors (e.g., as described with respect to FIG. 2) or application specific chips, and/or by other components of FIG. 2.

Therefore, according to the above, some examples of the disclosure are directed to a method (e.g., method 950 of FIG. 9L) including at an electronic device in communication with one or more displays and one or more input devices, concurrently displaying (952), via the one or more displays a first three-dimensional (3D) object; and a first portion of a 3D model of a second object inside the first 3D object, wherein the first portion of the 3D model is displayed at a first level of visual prominence, without displaying a second portion, different from the first portion, of the 3D model of the second object, wherein the first portion of the 3D model of the second object corresponds to a first volume of the 3D model of the second object within the first 3D object, and wherein the second portion of the 3D model of the second object corresponds to a second volume of the 3D model of the second object that would extend beyond a boundary of the first 3D object were the second portion displayed. The method 950 includes while concurrently displaying the first 3D object and the first portion of the 3D model of the second object inside the first 3D object, the first portion of the 3D model of the second object at the first level of visual prominence without displaying the second portion of the 3D model of the second object, detecting (954), via the one or more input devices, a first selection input including a movement component, the first selection input directed to the three-dimensional model of the second object. The method 950 includes in response to detecting the movement component, rotating (956) the 3D model of the second object about an axis associated with the 3D model of the second object based on the movement component of the selection input, including concurrently displaying, via the one or more displays, the first 3D object, a first respective portion of the 3D model of the second object inside the first 3D object, and a second respective portion, different from the first respective portion, of the 3D model of the second object outside of the first 3D object at a second level of visual prominence that is different from the first level of visual prominence.

Additionally or alternatively, in some examples, the second level of visual prominence is less than the first level of visual prominence.

Additionally or alternatively, in some examples, the second level of visual prominence is greater than the first level of visual prominence.

Additionally or alternatively, in some examples, rotating the 3D model of the second object about the axis includes rotating by a first amount, and the method 950 includes after rotating the 3D model of the second object about the axis, detecting, via the one or more input devices, conclusion of the first selection input, and in response to detecting the conclusion of the first selection input, concurrently displaying, via the one or more displays, the first 3D object and the first respective portion of the 3D model of the second object inside the first 3D object, without displaying the second respective portion of the 3D model of the second object. The first respective portion of the 3D model of the second object corresponds to a first respective volume of the 3D model of the second object that is within the first 3D object when the conclusion of the first selection input is detected, and the second respective portion of the 3D model of the second object corresponds to a second respective volume of the 3D model of the second object that would extend beyond a boundary of the first 3D object were the second respective portion displayed when the conclusion of the first selection input is detected. Additionally or alternatively, in some examples, the first respective volume is less than the first volume. Additionally or alternatively, in some examples, the first respective volume is greater than the first volume. Additionally or alternatively, in some examples, the first respective volume is equal to the first volume and the first respective portion is different from the first portion.

Additionally or alternatively, in some examples, the method 950 includes in response to detecting the movement component, displaying, via the one or more displays, the first respective portion of the 3D model of the second object that is inside the first 3D object at the first level of visual prominence.

Additionally or alternatively, in some examples, the method 950 includes in response to detecting the movement component, displaying, via the one or more displays, the second respective portion of the 3D model of the second object that is outside of the first 3D object at the second level of visual prominence.

Additionally or alternatively, in some examples, the method 950 includes comprising in response to detecting the movement component, displaying the first respective portion of the 3D model of the second object is the first level of visual prominence and after displaying the first respective portion of the 3D model of the second object is the first level of visual prominence, in accordance with a determination that the first respective portion of the 3DD model of the second object is rotated by a first respective amount, displaying the first respective portion of the 3D model of the second object at the second level of visual prominence.

Additionally or alternatively, in some examples, the method 950 includes displaying, via the or more displays, a user interface element indicative of an orientation of the 3D model of the second object.

Additionally or alternatively, in some examples, the 3D model of the second object is asymmetrical about the axis, in accordance with a determination that rotating the 3D model of the second object about the axis includes a first amount of rotation, the 3D model of the second object has a first shape from a viewpoint of the electronic device, and in accordance with a determination that rotating the 3D model of the second object about the axis includes a second amount of rotation that is different from the first amount of rotation, the 3D model of the second object has a second shape that is different from the first shape from the viewpoint of the electronic device that is different from the first shape.

Additionally or alternatively, in some examples, the first 3D object is of a first respective volume, and the method 950 includes while concurrently displaying the first 3D object having the first respective volume and a first amount of the 3D model of the second object inside the first 3D object, detecting, via the one or more input devices, a second selection input including a second movement component, the second selection input directed to a user interface element associated with the first 3D object, and in response to detecting the second movement component, concurrently updating display of the first 3D object to have a second respective volume that is different from the first respective volume and changing an amount of the 3D model of the second object that is displayed inside the first 3D object to be a second amount, different from the first amount of the 3D model of the second object, based on the second selection input (e.g., based on an amount of movement associated with the second movement component). Additionally or alternatively, in some examples, the electronic device includes a head-mounted display system.

As described above, one aspect of the present technology is the gathering and use of data available from various sources to improve XR experiences of users. The present disclosure contemplates that in some instances, this gathered data may include personal information data that uniquely identifies or can be used to contact or locate a specific person. Such personal information data can include demographic data, location-based data, telephone numbers, email addresses, social media identities or usernames, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to improve an XR experience of a user. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed examples, the present disclosure also contemplates that the various examples can also be implemented without the need for accessing such personal information data. That is, the various examples of the present technology are not rendered inoperable due to the lack of all or a portion of such personal information data. For example, an XR experience can be generated by inferring preferences based on non-personal information data or a bare minimum amount of personal information, such as the content being requested by the device associated with a user, other non-personal information available to the service, or publicly available information.

The foregoing description, for purpose of explanation, has been described with reference to specific examples. However, the illustrative discussions above are not intended to be exhaustive or to limit the disclosure to the precise forms disclosed. Many modifications and variations are possible in view of the above teachings. The examples were chosen and described in order to best explain the principles of the disclosure and its practical applications, to thereby enable others skilled in the art to best use the disclosure and various described examples with various modifications as are suited to the particular use contemplated.

1. A method comprising:
at an electronic device in communication with one or more displays and one or more input devices, including a camera:
presenting, via the one or more displays, a view of a physical environment of the electronic device from a viewpoint of the one or more displays in the physical environment, the view of the physical environment including:
an external view of a physical object; and
a first physical tool, different from the camera;
while presenting the view of the physical environment, displaying, via the one or more displays, a first user interface including video feed from the camera;
detecting that a respective part of the first physical tool is at a location associated with the physical object that is absent from the video feed from the camera; and
in response to detecting that the respective part is at the location associated with the physical object that is absent from the video feed from the camera, displaying, via the one or more displays, a representation of the respective part of the first physical tool.

2. The method of claim 1, wherein the first physical tool includes a tip and a portion other than the tip, and the respective part is the tip.

3. The method of claim 1, wherein the first physical tool includes a tip and a portion other than the tip, and the respective part is the tip and the portion other than the tip.

4. The method of claim 1, wherein the first physical tool includes a tip and a portion other than the tip, and the respective part is the portion other than the tip.

5. The method of claim 1, comprising:
while displaying, via the one or more displays, the representation of the respective part of the first physical tool, detecting that the respective part of the first physical tool is at a location associated with the physical object that is in the video feed from the camera; and
in response to detecting that the respective part of the first physical tool is at that location associated with the physical object that is in the video feed from the camera, ceasing displaying at least a portion of the representation of the respective part of the first physical tool.

6. The method of claim 1, wherein a length of the representation of the respective part of the first physical tool is based on a length of the respective part of the first physical tool.

7. The method of claim 1, wherein a length of the representation of the respective part of the first physical tool is based on a distance between a position within a field of view of the camera and the respective part of the first physical tool.

8. The method of claim 7, comprising:
detecting an input that changes the distance between the position within the field of view of the camera and the respective part of the first physical tool; and
in response to detecting the input that changes the distance between the position within the field of view of the camera and the respective part of the first physical tool:
in accordance with a determination that the input increases the distance between the position within the field of view of the camera and the respective part of the first physical tool, increasing the length of the representation of the respective part of the first physical tool; and
in accordance with a determination that the input decreases the distance between the position within the field of view of the camera and the respective part of the first physical tool, decreasing the length of the representation of the respective part of the first physical tool.

9. An electronic device comprising:
one or more processors;
memory; and
one or more programs stored in the memory and configured to be executed by the one or more processors, wherein the electronic device is in communication with one or more displays and one or more input devices, including a camera, and wherein the one or more programs include instructions for:
presenting, via the one or more displays, a view of a physical environment of the electronic device from a viewpoint of the one or more displays in the physical environment, the view of the physical environment including:
an external view of a physical object; and
a first physical tool, different from the camera;
while presenting the view of the physical environment, displaying, via the one or more displays, a first user interface including video feed from the camera;
detecting that a respective part of the first physical tool is at a location associated with the physical object that is absent from the video feed from the camera; and
in response to detecting that the respective part is at the location associated with the physical object that is absent from the video feed from the camera, displaying, via the one or more displays, a representation of the respective part of the first physical tool.

10. The electronic device of claim 9, wherein the representation of the respective part of the first physical tool is displayed outside of the first user interface that includes the video feed from the camera.

11. The electronic device of claim 10, wherein displaying the representation of the respective part of the first physical tool outside the first user interface includes displaying the representation of the respective part of the first physical tool at a location that is based on a spatial arrangement between the first physical tool and the camera in the physical environment.

12. The electronic device of claim 9, wherein the one or more programs include instructions for: while the respective part of the first physical tool is in the video feed from the camera, displaying, via the one or more displays and in the first user interface, an indication of a distance between the respective part of the first physical tool and a respective internal part of the physical object.

13. The electronic device of claim 12, wherein:
in accordance with a determination that the distance is a first distance, the indication has a first appearance; and
in accordance with a determination that the distance is a second distance, different from the first distance, the indication has a second appearance, different from the first appearance.

14. The electronic device of claim 9, wherein the camera is a laparoscopic camera, the physical object is a body of a human, and the first physical tool is a surgical instrument.

15. The electronic device of claim 9, wherein the first physical tool includes a tip and a portion other than the tip, and the respective part is the tip.

16. The electronic device of claim 9, wherein the first physical tool includes a tip and a portion other than the tip, and the respective part is the tip and the portion other than the tip.

17. A non-transitory computer readable storage medium storing one or more programs, the one or more programs comprising instructions, which when executed by one or more processors of an electronic device that is in communication with one or more displays and one or more input devices, including a camera, cause the electronic device to:
present, via the one or more displays, a view of a physical environment of the electronic device from a viewpoint of the one or more displays in the physical environment, the view of the physical environment including:
an external view of a physical object; and
a first physical tool, different from the camera;
while presenting the view of the physical environment, display, via the one or more displays, a first user interface including video feed from the camera;
detect that a respective part of the first physical tool is at a location associated with the physical object that is absent from the video feed from the camera; and
in response to detecting that the respective part is at the location associated with the physical object that is absent from the video feed from the camera, display, via the one or more displays, a representation of the respective part of the first physical tool.

18. The non-transitory computer readable storage medium of claim 17, wherein the first physical tool includes a tip and a portion other than the tip, and the respective part is the tip.

19. The non-transitory computer readable storage medium of claim 17, wherein the first physical tool includes a tip and a portion other than the tip, and the respective part is the tip and the portion other than the tip.

20. The non-transitory computer readable storage medium of claim 17, wherein the first physical tool includes a tip and a portion other than the tip, and the respective part is the portion other than the tip.

21. The non-transitory computer readable storage medium of claim 17, wherein the instructions, when executed, cause the electronic device to:
while displaying, via the one or more displays, the representation of the respective part of the first physical tool, detect that the respective part of the first physical tool is at a location associated with the physical object that is in the video feed from the camera; and
in response to detecting that the respective part of the first physical tool is at that location associated with the physical object that is in the video feed from the camera, cease displaying at least a portion of the representation of the respective part of the first physical tool.

22. The non-transitory computer readable storage medium of claim 17, wherein a length of the representation of the respective part of the first physical tool is based on a length of the respective part of the first physical tool.

23. The non-transitory computer readable storage medium of claim 17, wherein a length of the representation of the respective part of the first physical tool is based on a distance between a position within a field of view of the camera and the respective part of the first physical tool.

24. The non-transitory computer readable storage medium of claim 23, wherein the instructions, when executed, cause the electronic device to:
detect an input that changes the distance between the position within the field of view of the camera and the respective part of the first physical tool; and
in response to detecting the input that changes the distance between the position within the field of view of the camera and the respective part of the first physical tool:
in accordance with a determination that the input increases the distance between the position within the field of view of the camera and the respective part of the first physical tool, increase the length of the representation of the respective part of the first physical tool; and
in accordance with a determination that the input decreases the distance between the position within the field of view of the camera and the respective part of the first physical tool, decrease the length of the representation of the respective part of the first physical tool.
